(19) United States
(12) Patent Application Publication
(10) Pub. No.: US 2019/0103179 A1
(43) Pub. Date: Apr. 4, 2019

(54) AUTHENTICATED COLLECTION OF MEDICATION DATA AND USE THEREOF IN ADDRESSING NONADHERENCE

(71) Applicant: Kali Care, Inc., Mountain View, CA (US)

(72) Inventors: Sina Fateh, Mountain View, CA (US); Philippe Cailloux, Sunnyvale, CA (US); Navid Nick Afsarifard, Atherton, CA (US)

(21) Appl. No.: 16/137,270

(22) Filed: Sep. 20, 2018

Related U.S. Application Data
(60) Provisional application No. 62/568,243, filed on Oct. 4, 2017.

(51) Int. Cl. G16H 20/10

(52) U.S. Cl. CPC G16H 20/10

(57) ABSTRACT

Sensors may be engaged with a container to provide data on how the container is manipulated. Data traces from those sensors exhibit signatures of events such as opening the container, dispensing medication, etc. Such medication events and contextual events provide authenticated data on the use of medication, as distinct from self-reported or predicted/modeled data. Multiple signatures may be combined to determine a confidence level for whether a medication has been taken, and/or aspects such as when, how, how much, etc. A container may be retrofitted by engaging a shoe containing sensors with the bottom of the container. Authenticated data may support improved selection and oversight of research subjects to reduce cost, time, etc. of clinical trials. Authenticated data also may support improved patient outcome. Pools of subjects and patients may be developed to inform selection of subjects and prescription of medications to patients.

### CROSS-REFERENCE TO RELATED APPLICATIONS

This application claims priority to U.S. Provisional Application Ser. No. 62/568,243 filed Oct. 4, 2017, which is incorporated herein by reference for all purposes.

### FIELD OF THE INVENTION

Various embodiments concern acquisition of information indicating the use of medication. More particularly, various embodiments relate to “smart” systems for detecting data regarding medication being dispensed (or some other therapeutic process being carried out) without requiring self-reporting by the user, for example based on behaviors of the container for the medication. Various embodiments also relate to the application of such data in diminishing or counteracting nonadherence with a prescribed regimen for the medication.

### BACKGROUND

A substantial portion of medications are not taken as prescribed. By some estimates, in clinical practice up to 50% or more of medications either may not be taken at all or may be taken with significant deviations from what is prescribed for the patient. For example, doses of a medication may be skipped, the medication may not be taken at the right intervals, at the right times, in the right dose, applied in the correct manner, etc. Such deviation from a prescribed medication regimen may be referred to broadly as “nonadherence”. Nonadherence to prescribed medication regimens may have dramatic negative effects on health and/or healthcare costs, whether considering individuals or societies collectively.

Nonadherence may be even more common in clinical research, wherein some estimates indicate nonadherence of up to 70% or more. Nonadherence in a research context also presents other potential concerns. For example, testing of new medications typically may include efforts to determine the effectiveness of the medication, what side effects occur, how severe those side effects may be, in what fraction of the population those side effects occur, etc. Thus, nonadherence in a research setting may distort the basic understanding of a medication, e.g., if a medication is in fact highly effective if taken as prescribed but ineffective or dangerous if not taken properly, poor adherence within a clinical trial may result in data showing that the medication is not effective (when the actual problem is that it was not taken correctly).

One matter complicating issues related to nonadherence is that reliable data on the existence, degree, and form(s) of nonadherence present may be difficult to acquire. Whether for an individual, a larger population, or even a carefully selected and/or monitored group such as the subjects in a clinical trial, authentic data on how much nonadherence is taking place, among whom, and in what forms (e.g., missing doses, taking the medication incorrectly, etc.) may not be available through conventional sources. Typically, key information on adherence may be obtained through self-reporting by patients and/or test subjects. However, self-reporting also may be unreliable. Put bluntly, if patients do not reliably take a medication, patients' reports of taking that medication also may be unreliable. Thus, in practice it may not even be known how much nonadherence is taking place (beyond estimates), much less what the specific impacts of nonadherence may be in a given case, without authenticated data.

### BRIEF DESCRIPTION OF THE SEVERAL VIEWS OF THE DRAWINGS

Various objects, features, and characteristics will become more apparent to those skilled in the art from a study of the following Detailed Description in conjunction with the appended claims and drawings, all of which form a part of this specification. While the accompanying drawings include illustrations of various embodiments, the drawings are not intended to limit the claimed subject matter.

FIG. 1A through FIG. 1T depict an example series of events in an instance of administering a medication, in profile view.

FIG. 2 depicts an example series of orientation and pressure measurements over time, corresponding with events in an instance of administering a medication in the form of eye drops.

FIG. 3 depicts an example method of determining adherence to a medication regimen, in flow chart form.

FIG. 4 through FIG. 10 depict example traces for orientation and pressure over time, corresponding with events in an instance of administering a medication in the form of eye drops.

FIG. 11 through FIG. 16 show example systems for determining adherence to a medication regimen, in cross-section view.

FIG. 17 through FIG. 31 show example systems for determining adherence to a medication regimen with particular regard to a shoe therefor, in cross-section view.

FIG. 32 shows an example system for determining adherence to a medication regimen with a base therefor, in cross-section view.

FIG. 33 shows example arrangements for engaging a shoe in a single configuration, in top-down view.

FIG. 34 through FIG. 37 show example systems for determining adherence to a medication regimen, in schematic view.

FIG. 38 through FIG. 49 show example methods for determining adherence to a medication regimen, in flow chart form.

FIG. 50 through FIG. 53 show example arrangements for considering, displaying, and/or configuring adherence information.

FIG. 54 through FIG. 62 show example methods for considering and implementing authenticated medication and adherence information for improved research and treatment, in flow-chart form.

FIG. 63 is a block diagram illustrating an example of a processing system in which at least some operations described herein can be implemented.

The figures depict various embodiments described throughout the Detailed Description for the purposes of illustration only. While specific embodiments have been shown by way of example in the drawings and are described in detail below, the technology is amenable to various modifications and alternative forms. The intention is not to limit the technology to the particular embodiments described. Accordingly, the claimed subject matter is intended to cover all modifications, equivalents, and alternatives falling within the scope of the technology as defined by the appended claims.

### DETAILED DESCRIPTION OF THE INVENTION

Various embodiments are described herein that relate to the collection of data regarding the administration of medication, and the use of such data in addressing nonadherence to a medication regimen. For example, sensors may collect data about whether, how, and when pressure is applied to a container of eye drop medication, whether, how, and when the container is moved, and so forth. Data may be acquired as “traces”, such as a series of pressure measurements indicating the pressure that is applied to the container over time along with a series of orientation measurements indicating the orientation of that container over time. Through evaluation of these traces, signatures that are characteristic of various actions such as picking up the container, removing the cap, squeezing out drops of medication into a subject's eyes, etc. may be identified. Those signatures in turn may be evaluated to determine whether the medication was administered, or whether the pressure and orientation (or other properties) traces reflect something other than an instance of administering medication, such as casual handling of the container, motion with a pocket or bag, etc. In such manner, it may be determined whether and when a medication is administered, and/or other information related to the use of the medication.

Typically, though not necessarily, signatures may include features in two or more traces considered in concert. For example, a change in orientation followed by a spike in pressure may be interpreted as a signature characteristic of tipping a bottle of eye medication over a patient's eye and squeezing out a drop of medication into the patient's eye. While at least in principle single-trace arrangements may reveal at least some information, utilizing two or more traces in identifying signatures of events in administering a medication may be more reliable and/or less prone to errors. For example, a single pressure trace may show spikes when a patient stands or sits if the bottle is in his or her pocket, spikes that may not be distinct from a pressure spike associated with squeezing out a drop. This is not to imply that two separate properties necessarily must be measured, however; two types of measurement of pressures may, for example, reveal signatures distinguishing between “pocket squeezing” and administering an eyedrop.

Conventional approaches to determining adherence to a medication regimen rely heavily if not entirely upon self-reporting by patients. However, self-reporting of adherence is notoriously unreliable. Patients tend to report high adherence regardless of their actual level of compliance. Patients may be unaware of their actual degree of adherence, or may simply report adherence for social or psychological reasons (e.g. to appear agreeable, to avoid criticism, etc.). Deliberate deception is also possible. Moreover, even otherwise reliable self-reporting may be limited in precision, e.g., a patient may record a medication as having been taken at 3 PM when in fact the medication was taken at 3:14 PM. (Such imprecision may not be an issue for all medications, but certain medications are extremely sensitive with regard to timing, dosage, etc.) Objective information regarding how and when a medication is taken may prove more reliable than self-reporting.

Such objective data on whether a medication is being taken as prescribed may be useful for many reasons. For example, the well-being of a patient may be directly affected by their degree of adherence to a medical treatment regimen. Effectively results with a medication may depend on taking that medication at a specified dose and according to a specified schedule, in order to maximize the effectiveness of the medication and/or minimize side effects. While some medications may remain safe and effective even with considerable deviation from a prescribed regimen, this is not true of all medications. For example, glaucoma medications may exhibit greatly reduced effects if a schedule for taking them is not closely followed, if not taken in the proper amounts, etc. As another example, opiate pain relievers may pose an increasing risk of addiction and side effects if used too often, for too long, or in too great a dose. Objective data regarding adherence thus may directly improve medical outcomes, for example by enabling patients to track and improve their adherence.

As another example, the ability of a physician (or other medical professional) to supervise and guide the treatment of a patient also may be affected by the physician's knowledge of how and to what degree the patient may be deviating from a prescribed regimen. Not all medications affect all patients uniformly; patient A may see a strong benefit from a given drug, while patient B experiences little or no benefit, and patient C experiences side effects so severe as to outweigh any benefits. However, low benefit and/or severe side effects also may result from taking the drug improperly. It may be difficult or impossible to distinguish from the outcome alone (the effects and side effects) whether the problem is the drug itself or that the patient is not adhering sufficiently to the prescribed regimen. Thus, objective data regarding adherence also may indirectly improve medical outcomes, for example by providing physicians with the information needed to successfully manage the patient's care.

As yet another example, clinical trials or other evaluations typically may be conducted precisely because the properties of a medication are not well-understood. Such trials typically may be performed in order to determine the medication's therapeutic effects (if any), side effects and severity thereof, sensitivity to variations in dosage or timing, etc. If test subject adherence is unknown, or is known only with low confidence (e.g., from self-reporting), this may obscure the actual properties of the medication. In principle it may be possible to compensate low and/or uncertain adherence, but typically such compensation is not free. For example, using statistical analysis on trials with a larger number of test subjects, extending over a longer period, etc. may accommodate lack of adherence, but also may increase the cost of the trial. Identifying high-adherence test subjects thus may improve the reliability of clinical trials, and/or reduce their size, duration, cost, etc. However, even if adherence is low, it may still be possible to efficiently correct data statistically (or otherwise) if the degree and nature of nonadherence is at least known. Thus, objective data regarding adherence may improve testing of medications, for example by enabling selection of high-adherence test subjects and/or correcting for known patterns of non-adherence.

### Terminology

Brief definitions of terms, abbreviations, and phrases used throughout this application are given below.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described that may be exhibited by some embodiments and not by others. Similarly, various requirements are described that may be requirements for some embodiments but not others.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” “engaged”, or any variant thereof, means any connection, coupling, or engagement, either direct or indirect, between two or more elements; the coupling or connection between the elements can be physical, logical, or a combination thereof, and may be readily removable or essentially permanent. For example, two devices may be coupled directly to one another, or via one or more intermediary channels or devices, and/or may be coupled via removable plugs or integrated together into a solid structure. Devices may also be coupled in such a way that information can be passed there between, while not sharing any physical connection with one another.

Additionally, the words “herein,” “above,” “below,” and words of similar import, when used in this application, shall refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. If the specification states a component or feature “may,” “can,” “could,” or “might” be included or have a characteristic, that particular component or feature is not required to be included or have the characteristic.

The term “module” refers broadly to software, hardware, and/or firmware components. Modules are typically functional components that can generate useful data or other output using specified input(s). A module may or may not be self-contained. A software program or application may include one or more modules.

The terminology used in the Detailed Description is intended to be interpreted in its broadest reasonable manner, even though it is being used in conjunction with certain examples. The terms used in this specification generally have their ordinary meanings in the art, within the context of the disclosure, and in the specific context where each term is used. For convenience, certain terms may be highlighted, for example using capitalization, italics, and/or quotation marks. The use of highlighting has no influence on the scope and meaning of a term; the scope and meaning of a term is the same, in the same context, whether or not it is highlighted. It will be appreciated that the same element can be described in more than one way.

Consequently, alternative language and synonyms may be used for some of the terms discussed herein. Although synonyms for certain terms may be provided, special significance is not to be placed on whether or not a term is elaborated or discussed herein. A recital of one or more synonyms does not exclude the use of other synonyms. The use of examples anywhere in this specification, including examples of any terms discussed herein, is illustrative only and is not intended to further limit the scope and meaning of the disclosure or of any exemplified term. Likewise, the disclosure is not limited to various embodiments given in this specification.

System Topology Overview

Adherence to a prescribed medication regimen typically may be determined through self-reporting by the patients and/or test subjects taking the medication. However, among other concerns a patient who does not reliably take a medication also may not reliably self-report on their taking of that medication. This does not necessarily imply ill intent; human error may take place despite good faith. Indeed, factors contributing to unintentional nonadherence may also contribute to poor self-reporting of nonadherence; for example, a patient who forgets to take a prescribed medication also may forget to record if, when, and how they took that medication.

Regardless of the reason(s) for nonadherence and/or or poor-quality self-reporting data regarding adherence, it may be useful to obtain adherence data that is “authentic” or “authenticated”, that is, that does not rely on the goodwill and memory of the patient to reflect whether, when, and how a medication is being taken. Authentic adherence data may be obtained by using sensors to collect data indicating whether a medication has been taken, at what time, etc. Such sensors and/or related systems may be automated to at least some degree, in which case authenticated data may be (but is not required to be) acquired autonomously, without requiring users or other persons to actively record data. For example, an approach may be based on the “internet of things”, by engaging sensors with a medication container so as to yield a “smart bottle” that may record data as needed, or even continuously.

Prior to describing approaches for obtaining such authenticated adherence data, it may be illuminating to summarize certain features regarding authenticated adherence data. (Not all embodiments necessarily will exhibit all such features, nor is it required for any particular embodiment to include a particular feature.)

It may be possible to identify the actual medication event of taking a medication—for example, expelling a droplet of eye medication from a squeeze bottle into a patient's eye—from sensor values at a given moment in time. For example, if a squeeze bottle were equipped with an orientation sensor and a pressure sensor, and at some moment the bottle were inclined (say) 120 degrees from vertical and a level of force determined as sufficient force to squeeze out a droplet were also present, those momentary conditions could be defined/interpreted as a medication event (that is, taking the medication).

However, it may also prove useful to consider data not merely as individual or discrete data points—e.g., the orientation and pressure at a single moment—but over some period of time. For example, rather than merely watching for an instant when the orientation is within a given range and the pressure exceeds a given minimum, it may be advantageous to collect a running trace of orientation and/or pressure over time, and to determine whether some portion of that trace exhibits a characteristic “signature” that corresponds with real-world events. The shape of pressure and orientation traces (e.g. as curves plotted against time) may reveal, for example, whether medication truly is being dispensed, or whether the bottle simply assumed a particular orientation in a pocket and was momentarily pinched by something else in the pocket such as the patient's car keys. Thus, consideration of traces to detect signatures therein, rather than discrete values only, may help distinguish false positives from real medication events. Considering traces also may help avoid false negatives. For example, may not orient an eyedrop bottle to a consistent angle, and certain bottles may require variable pressure to extrude a droplet (e.g. a harder squeeze may be needed as the bottle empties of incompressible liquid and fills with compressible air). Simply watching for a specified orientation/pressure at a moment in time may not reveal real uses of the medication, while the shape, size ratio (width to height, etc.), or other properties of features in traces may do so. In addition, shapes of data traces may reveal other information; if a user is hesitating when squeezing out a droplet, the pressure trace may exhibit a different shape than if the user were not hesitating. Hesitation may in turn suggest difficulty in administering the eyedrop, reluctance to administer the eyedrop, and/or other issues which might be addressed to improve outcomes (for example the patient's care provider may allay fears, address side effects, specify an easier-to-use bottle, etc.).

It also may be informative to examine data traces for signatures of events other than the medication event itself. For example, if a medication bottle has a cap, removing that cap also may leave a signature in the data traces. Removing the cap may be referred to as a “contextual event” (as distinct from a medication event); removing the cap is not the medical treatment, but the act of removing the cap may provide evidence that the medication is being taken. For example, if the traces exhibit no signature that the cap was removed, then an orientation/pressure traces that resemble a signature for squeezing out a droplet may be a false positive, not a medication event. (A distinction may be made between “events” that happen in the world and “signatures” that are shapes/forms/other relationships within data traces that may be characteristic of an event.) As another example, if the medication is to be shaken before use, but the traces show no signature corresponding to the bottle being shaken, this may indicate a form of nonadherence (i.e., the patient is not mixing the medication properly).

Given sufficiently rich data—for example, shapes of signatures in traces and/or signatures of contextual events in combination with (or even in place of) medication events—it may be useful to consider a more nuanced (and potentially more informative) approach to determining adherence than simply “yes/no” or “adhering/nonadhering”. For example, a confidence level may be determined, gauging how likely it is that the medication has actually been taken (as opposed to there being a false positive). If characteristic signatures are found in data traces showing the cap of a bottle being removed, the bottle being tilted, and the bottled being squeezed to expel droplets, those signatures may contribute to a high confidence that the subject has taken the medication. However, if one or more signatures exhibited an unusual shape, was missing, or was out of order (e.g., if cap removal followed the bottle being squeezed), confidence in a medication instance may be lower. Adherence too may be gauged, such as by determining an “adherence factor” that indicates how well a subject is adhering to a regimen. Multiple adherence factors may be determined, for example one that reflects the degree to which a medication is taken on time, one that reflects the degree to which the medication is taken using proper procedure (e.g. shaking before use), etc.

In acquiring data without necessarily relying on self-reporting, and/or in acquiring data that is sufficiently rich as to enable functional interpretation of signatures rather than detecting single-point values or other simplistic features, information regarding actual adherence may be thought of—and/or utilized—as being authentic or authenticated. Where self-reported information on medication adherence may be subject to human error (just as human error may cause nonadherence itself), acquiring and considering authenticated data, such as automated sensor data, may facilitate evaluations that are (in colloquial terms) “more real”, in that the sensor data may more authentically reflect the actual use of the medication and the degree to which that use matches the prescribed regimen therefor.

Such authenticated data may in turn facilitate other useful functions. For example, consider a patient who self-reports to their medical care provider that a medication is not effective. If the provider has no way to authenticate that self-report, lack of authentic information may limit the provider's ability to properly address the problem and improve the patient's outcome. If the patient really is adhering to the regimen, the lack of effect may be due to medical factors (e.g., this patient does not respond well to this medication, other factors such as diet, environment, etc. are affecting the outcome, etc.); in this case changing the medication regimen may be useful, such as by increasing the dose, changing to a different medication, etc. However, if the patient is not really adhering, the lack of effect may be due to nonadherence; in this case working with the patient to improve adherence may be useful. Note that such uncertainty may be present even if the patient is adhering perfectly, because the provider may have no way to know whether the patient's self-reports are accurate. Thus, access to authenticated adherence data may facilitate improvement of patient outcomes, by giving providers (and/or patients, etc.) access to more reliable information. Access to authenticated adherence data likewise may facilitate reduced costs, and/or have other benefits. For example, nonadherence to antibiotic prescriptions may contribute to the development of antibiotic-resistant strains of infectious microorganisms, thus improving adherence may at least help to mitigate certain long-term public health risks.

In addition, medical research may benefit from access to authenticated adherence data. For example, in a clinical trial to determine the effectiveness and/or side effects for a new medication, not all patients will necessarily take the medication as directed. Such nonadherence may obscure the performance of the medication, e.g., if the medication is highly effective if taken as directed but is not taken as directed, the trial may incorrectly indicate that the medication is ineffective. It may be possible to design a clinical trial to compensate for nonadherence, for example by using statistical techniques on the resulting clinical trial data. However, if the actual degree of nonadherence itself is not known, too much or too little compensation may be applied; thus a statistical “correction” may or may not be a correction at all. If the type of nonadherence is not known, statistical compensation likewise may not be effective. For instance, statistically compensating for patients taking the wrong dose when the problem is that patients are missing doses also may not constitute “correction”. Also, statistical correction may require or at least benefit from increasing the size, duration, etc. of a trial to provide more data with which to work, but larger, longer trials typically may be more expensive, more difficult to manage, etc. In addition, expanding trials may be undesirable or even impossible in practice, for example if the population of suitable test subjects is limited, if the time available for a clinical trial is limited, etc. Decreasing nonadherence in clinical trials (or other research) may facilitate more accurate results, enable smaller and/or shorter trials (with correspondingly lesser cost, etc.), and so forth. This may be achieved for example by selecting subjects based at least in part on their past adherence, by selecting subjects with personality traits that suggest high adherence, etc. However, even if nonadherence is not decreased, the availability of authenticated data on the degree and type of nonadherence in a clinical trial may enable valid conclusions with fewer subjects, shorter times, lower cost, etc.

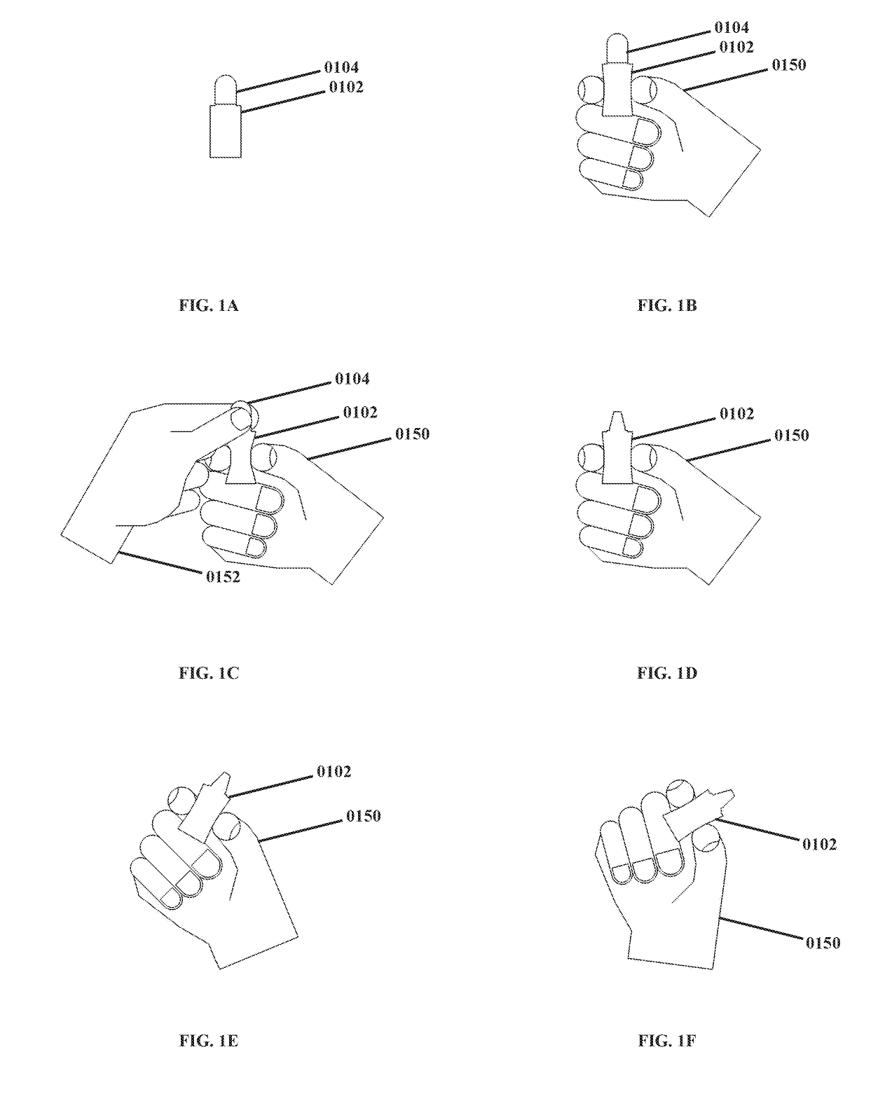

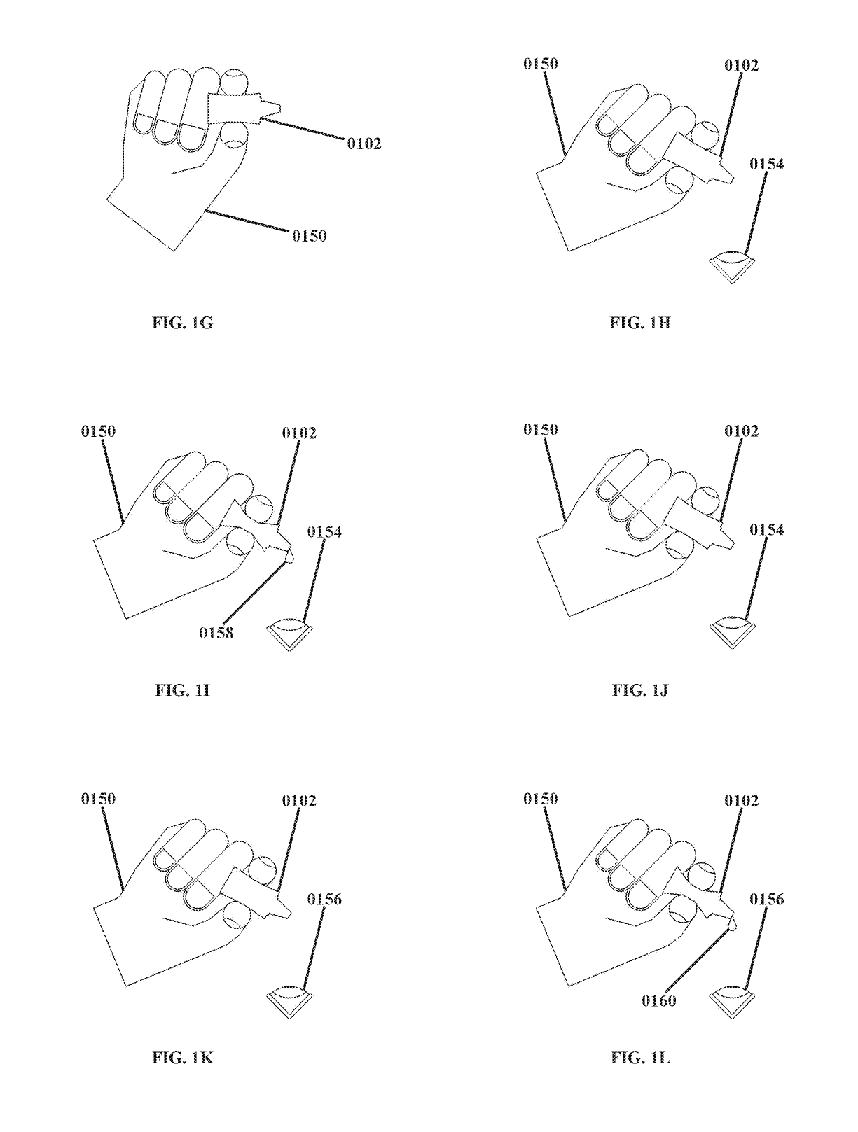

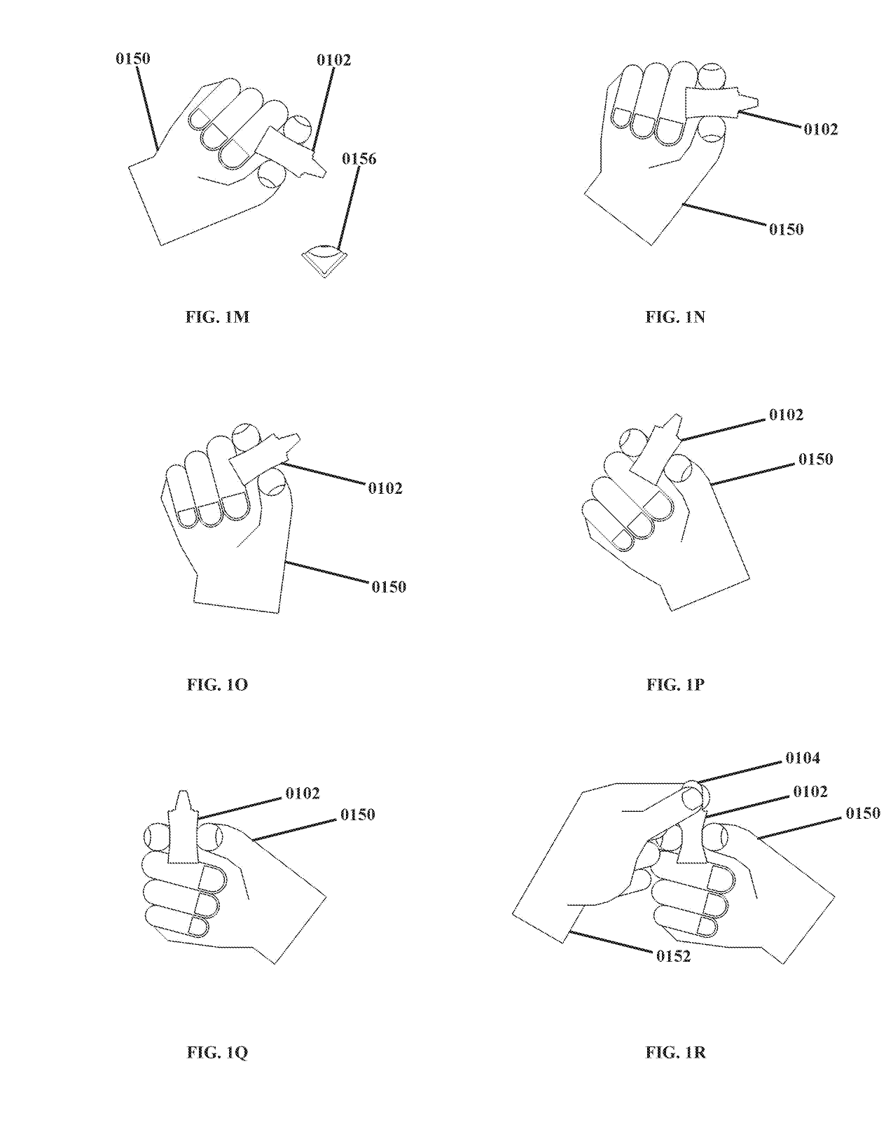

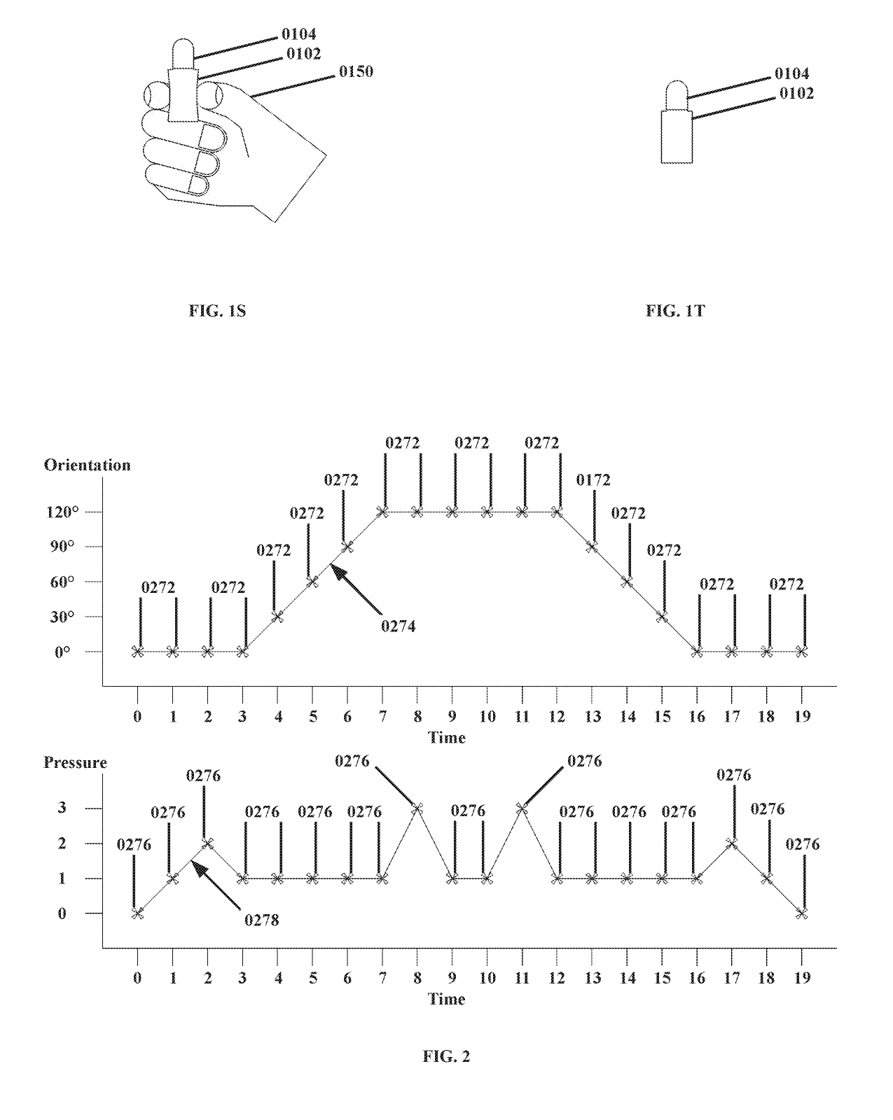

Taking a medication may at times be referred to as a singular act: taking a pill, injecting a medication, putting an eyedrop in an eye, etc. However, with reference to FIG. 1 collectively, it may useful instead or in addition to consider taking a medication as a series of events or as a process of some duration. While in strict terms only a small part of that process may be a medication event (i.e., actually taking the medication), other events may provide context for the medication event. As an example, for administering an eyedrop medication such contextual events may include varying orientations of the medication bottle over time, varying pressure applied the bottle over time, etc. Data regarding medication and/or contextual events may be collected and examined for characteristic data signatures that may correspond with such events; more regarding signatures is described with regard to FIG. 2. However, for explanatory purposes FIG. 1A through FIG. 1T are presented to illustrate example events associated with taking an eyedrop medication.

As a preliminary matter, some definition of terms may be useful at this point.

For purposes of discussion herein, data points are individual values for some property, for example as acquired from a sensor. Typically, a data point represents a single moment in time, or is otherwise discrete. Data traces are “curves” of multiple data points (though the shape need not be literally curved, and may be flat, jagged, etc.). Data curves typically show a property that changes over time (or along some other axis such as distance).

Actions that occur as part of or associated with the administration of a medication are referred to in places herein as “events”. Events that correspond with taking a medication may be referred to as “medication events”, while events that take place in relation to taking the medication may be referred to as “contextual events”. In colloquial terms, events (medication or contextual) may be considered to be “what's really happening” in a physical sense, for example a cap being removed from a bottle, the bottle being inclined, the bottle being squeezed to extrude a droplet, etc. FIG. 1 thus may be understood as illustrating events.

The term “signatures” refers to features of data traces that correspond with and/or are consistent with events. Thus' if an event is “what's happening”, a signature associated with that event may be for example a shape (or other feature) in a data trace that is characteristic of that event. Consequently, if the signature characteristic of an event is present in a data trace, it may be inferred that the event itself has taken place. FIG. 2 thus may be understood as illustrating signatures (e.g., in the shape of the traces shown therein).

Now once again with reference to FIG. 1A, a simple outline of a medication bottle 0102 is shown, such as a squeeze bottle for administering eye drops, with a cap 0104 thereon. As may be seen the medication bottle 0102 is at least approximately vertical, and nothing is shown to be contacting the bottle 0102 in such way as to apply significant pressure. The bottle 0102 may for example be sitting on a solid surface such as a table, shelf, etc. (not shown).

In FIG. 1B, a right hand 0150 is shown picking up the bottle 0102 and cap 0104. As may be seen, the bottle 0102 is slightly compressed where the forefinger and thumb of the right hand 0150 are gripping the bottle 0102, for example from pressure may applied to the bottle 0102 by the right hand 0150.

Moving to FIG. 1C, a left hand 0152 is also shown gripping the cap 0104 of the bottle 0102, while the right hand 0150 continues to grip the bottle 0150. Such an arrangement may occur for example if a person is removing (or replacing) the cap 0104 of the bottle 0102. In addition, as may be seen the bottle 0102 is more deformed in FIG. 1C than in (for example) FIG. 1B; the right hand 0150 may apply more pressure to the bottle 0102 when removing the cap 0104 than when merely grasping the bottle 0102.

FIG. 1D shows the right hand 0150 gripping the bottle 0102 with the cap (not shown) removed.

FIG. 1E through FIG. 1H show the hand 0150 moving in such manner as to tip the bottle 0102 away from the orientation (at least substantially vertical) shown in FIG. 1A through FIG. 1D. By FIG. 1H the bottle 0102 is inclined approximately 120 degrees from vertical. A first eye 0154 is also shown, with the bottle 0102 being arranged with the nozzle thereof disposed above the eye 0154. Such positioning of the bottle 0102 may serve to facilitate application of an eyedrop, for example.

Continuing in FIG. 1I, the hand 0150 is shown squeezing the bottle 0102 with such pressure that the bottle 0102 extrudes a droplet 0158 of medication. As may be seen, the deformation of the bottle 0152 is greater in FIG. 1I than in FIG. 1C where the cap 0104 was removed, and also greater than in other figures wherein the hand 0150 was merely grasping the bottle 0102. As noted previously with regard to FIG. 1C, more pressure may be applied by the hand 0150 to the bottle 0102 when opening then when grasping, and/or also when squeezing to dispense medication than when grasping.

Given the position of the bottle 0102 in FIG. 1I the first droplet 0158 may be presumed to fall downward into the eye 0154, such that the medication is administered. FIG. 1J then shows the hand 0150 holding the bottle 0102 over the eye 0154 after the droplet (not shown) has been administered; as may be seen the bottle 0102 in FIG. 1J is no longer so deformed as in FIG. 1I, as the hand 0150 has released pressure on the bottle 0102 so as not to administer additional droplets.

FIG. 1K, FIG. 1L, and FIG. 1M show views similar to those in FIG. 1H, FIG. 1I, and FIG. 1J, in that medication is again dispensed into an eye. However, as may be seen the bottle 0102 in FIG. 1K, FIG. 1L, and FIG. 1M is disposed over a second eye 0156 (e.g., the subject's left eye if the first eye 0154 was the right eye, or vice versa). Thus, though the bottle 0102 has not visibly changed orientation between FIG. 1J and FIG. 1K, the bottle 0102 may be understood to have moved laterally (e.g., from left eye to right or vice versa). Also similarly, in FIG. 1L a second droplet 0160 is dispensed into the second eye 0156.

Thus, as of FIG. 1M the medication in the bottle 0102 has been administered, one droplet 0158 and 0160 to each eye 0154 and 0156.

Continuing with FIG. 1N through FIG. 1Q, the hand 0150 progressively changes in orientation again, from the inclination of approximately 120 degrees from vertical as shown previously in FIG. 1M to a substantially vertical orientation in FIG. 1Q. FIG. 1R then shows the cap 0104 being replaced on the bottle 0102 by the left hand 0152 while the right hand 0150 holds the bottle 0102. In FIG. 1S the bottle 0102 is shown again with the cap 0104 in place, and in FIG. 1T the bottle 102 may be seen (along with the cap 0104) not being gripped by the hand 0150.

Thus collectively, FIG. 1A through FIG. 1T depict certain motions of a bottle 0102, pressures applied to that bottle 0102, etc., as medication is dispensed therefrom.

Turning now to FIG. 2, plots against time are depicted for a series of orientation values 0272 (not uniquely identified) for a medication container, and for a series of pressure values 0276 (also not uniquely identified) applied to the medication container. As may be seen, traces 0274 and 0278 are also shown illustrating the overall “curve” of orientation and applied pressure over time.

The orientation values 0272 are plotted as degrees of inclination from vertical ranging from 0° (i.e., vertical) to 120°, against time intervals ranging from 0 to 19. The time values are to at least some degree abstractions; the numbers shown for time do not necessarily represent seconds, minutes, etc. nor even necessarily uniform intervals. Rather, the values 0 through 19 represent events happening in sequence over time.

The pressure values 0276 are plotted as magnitudes of applied pressure ranging from 0 to 3, against time intervals ranging from 0 through 19. As noted with regard to the orientation values 0272, the time intervals for the pressure values 0276 represent events happening in sequence over time, without necessarily corresponding to fixed intervals, etc. Likewise, the pressure magnitudes 0 through 3 represent various degrees of pressure, but do not necessarily correspond to applied forces in Newtons, etc.

For illustrative purposes, the orientation vs. time and pressure vs. time plots in FIG. 2 are presented as corresponding with events associated with administering an eyedrop medication as illustrated in FIG. 1A through FIG. 1T. The time of 0 in FIG. 2 may be seen as reflecting conditions comparable to FIG. 1A, sequentially through the time of 19 in FIG. 2 which may be seen as reflecting conditions comparable to FIG. 1T. Thus in comparing FIG. 2 with FIG. 1A through FIG. 1T, certain aspects of the orientation and pressure traces 0274 and 0278 may be observed as corresponding with dispensing a medication as shown in FIG. 1A through FIG. 1T.

For example, in time intervals 0 through 3, the orientation trace 0274 remains substantially flat at 0° inclination from vertical, corresponding with the bottle 0102 in FIG. 1 remaining vertical in FIG. 1A through FIG. 1D. The orientation trace 0274 then inclines upward from times 3 through 7, corresponding with the bottle 0102 in FIG. 1 being inclined from FIG. 1D through FIG. 1H. The orientation trace 0274 is again substantially flat at 120° from time 7 through time 12, corresponding with the bottle 0102 remaining inclined over the subject's eyes from FIG. 1H through FIG. 1M. The orientation trace 0274 declines from times 12 through 16, corresponding with the bottle in FIG. 1 returning to 0° inclination from FIG. 1M through FIG. 1Q, and then remains flat at 0° from times 16 through 19 corresponding with the bottle 0102 remaining vertical in FIG. 1Q through FIG. 1T.

Similarly, the pressure trace 0278 begins at 0 at time 0 in FIG. 2, and increases to 2 at time 1 as the bottle 0102 is grasped in FIG. 1B. The pressure trace 0278 increases to 2 at time 2 as the cap 0104 is removed from the bottle 0102 in FIG. 1C, then decreases again to 1 at time 3 and remains so through time 7. The pressure trace 0278 rises from 1 to 3 at time 8, drops to 1 for times 9 and 10, and then rises again to 3 at time 11; this corresponds to the squeezing of the bottle 0102 to extrude droplets 0158 and 0160 in FIG. 1I and FIG. 1L. The pressure trace 0278 returns to 1 at time 12 and remains so through time 16, then increases to 2 at time 17 corresponding with the replacement of the cap 0104 in FIG. 1R. Thereafter the pressure trace 0278 decreases to 1 at time 18, and then decreases to zero at time 19 corresponding with the hand 0150 having released the bottle 0102 in FIG. 1T.

Strictly speaking, administering the medication (elsewhere referred to herein as a “medication event”) occurs at times 8 and 11 in FIG. 2, corresponding with squeezing out droplets 0158 and 160 in FIG. 1I and FIG. 1L. Thus, detecting the specific momentary conditions for orientation and pressure at times 8 and 11 may indicate that the medication has been administered. However, detecting traces of multiple orientation values and pressure values may also be useful. For example, as may be seen in FIG. 2 the pressure values 0276 at times 7, 8, and 9 and again at times 10, 11, and 12 exhibit visible rises and falls, presenting the appearance of spikes. That is, the pressure values 0276 begin relatively low, rise rapidly to a significantly higher level, and then decline rapidly again. Such an arrangement may be considered characteristic for squeezing a droplet from a bottle: the user applies strong pressure briefly, for just long enough to expel the droplet. Thus, the shapes of pressure values 0276 at times 7-9 and 10-12 may be understood as signatures for squeezing out a droplet. In the case of the arrangement shown, squeezing out a droplet is administering the medication, so the shapes of pressure values 0276 at times 7-9 and 10-12 may be considered specifically as medication signatures, i.e., signatures of a medication event.

Signatures may vary considerably in definition, and may be simple or complex. For example, a medication event signature for an arrangement similar to FIG. 1 and FIG. 2 could be defined simply as a rise to a pressure of at least 3 from a pressure of no more than 1, followed by a fall to a pressure of no more than 1 again. However, other features also may be part of a signature definition, such as a minimum and/or maximum total time for the rise and fall, a minimum and/or maximum dwell time at the pressure of 3, etc. In addition, although the arrangement shown in FIG. 2 is relatively low-resolution, and thus presents sharp bends in the pressure trace 0278 rather than curves, when sufficient resolution is available it may be suitable to consider the curvature, the height-to-width ratio, the slope and/or rate of change of slope, the degree of noise present (if any), and/or other factors. Signatures also may be defined at least partly in terms of other features present or absent in a trace; for example, a pressure peak may not be considered a signature of a droplet being squeezed out if previously there is no indication in the pressure trace 0278 that the cap was removed from the bottle. (However, it may also be suitable consider such contextual events in terms of separate contextual signatures, as described subsequently. In practice whether to consider opening the cap of a bottle as part of the signature of squeezing out a droplet, or as a separate signature unto itself, is at least to some degree a matter of analytical convenience. Embodiments are not limited with regard to such matters.)

In addition, signatures for an event may be defined so as to consider more than a single data trace. For example, during times 7-9 and 10-12 when the pressure trace 0278 exhibits the aforementioned spikes, the orientation trace 0274 also exhibits a plateau at an inclination of 120°. A definition as to whether times 7-9 and 10-12 are considered medication event signatures representing medication events may include both the pressure trace 0278 and the orientation trace 0274; for example, it may be required that the pressure trace 0278 spikes (e.g., as the bottle is squeezed) while the orientation trace 0274 reflects approximately constant inclination within some range such as 90° to 180° (e.g., as the bottle remains tilted to dispense the droplet). Thus such a signature would consider both traces 0274 and 0278 in cooperation; for the signature to be considered present, the orientation and pressure traces 0274 and 0278 both would have to exhibit defined properties (for example spikes in the pressure trace 0278 and a level but inclined orientation trace 0274).

Not all signatures must be defined to require or consider multiple traces. Certain event signatures for a given embodiment may consider multiple traces, while other event signatures do not. Also, signatures are not limited only to considering two traces; where three or more traces are available (e.g. from additional instruments, from multiple data feeds from a single sensor, etc.), a given signature may consider one, some, or all traces. Likewise, not all signatures must consider the same factors; one signature in a given embodiment may consider the curvature of a trace, while another signature considers height and width only. Other arrangements also may be suitable, and neither data traces nor signature definitions should be considered limited herein except as indicated.

Still with reference to FIG. 2, as noted certain portions thereof (e.g. times 7 to 9 and 10 to 12) may be considered as signatures for medication events. However, other signatures also may be defined and identified within traces, for events other than medication events. Such non-medication events may be referred to as contextual events, and signatures therefor referred to as contextual event signatures (or simply contextual signatures). While contextual event signatures may not represent the actual taking of a medication, nevertheless contextual event signatures may help confirm that a possible medication event signature is indeed so, may help rule out false positives, and/or may provide other useful information.

For example, as noted FIG. 1C shows the cap 104 of the bottle 102 being removed prior to administering eyedrops. Corresponding with FIG. 1C, time 2 in FIG. 2 shows a low spike in the pressure trace 0278 while the orientation trace 0274 shows the bottle as remaining vertical (inclined 0°). Meanwhile, times 1 and 3 immediately before and after show the orientation trace 0274 remaining unchanged while the pressure trace 0278 rises from time 1 to time 2 and falls again from time 2 to time 3. Collectively, the portions of the orientation and pressure traces 0274 and 0278 from times 1 to 3 in FIG. 2 may be considered to represent a signature for removing the cap 0104 from the bottle 0102 in FIG. 1C.

Typically an eyedrop bottle with a cap must have that cap removed or otherwise opened before eyedrops may be dispensed from that bottle. Thus, even though a bottle opening signature from times 1 to 3 in FIG. 2 may not itself show a medication being administered, nevertheless that bottle opening signature may help confirm that the subsequent eyedrop signatures at times 7 to 9 and 10 to 12 do correspond to real-world eyedrops being administered. Conversely, if the bottle opening signature were absent, then that absence may suggest that apparent eyedrop signatures are false positives. Similarly, times 3 to 7 may be considered a contextual signature of putting the bottle 0102 into position to expel eyedrops (the orientation trace 0274 shows increasing tilt, while the pressure trace 0278 shows approximately constant pressure).

It is noted that signatures may overlap, and that a given portion of a trace may be part of more than one signature. As in the preceding examples times 1 to 3 in FIG. 2 may form one signature, and times 3 to 7 may form another; time 3 thus is part of two signatures. While not required, such overlap also is not excluded.

In addition, other information besides whether the medication is taken may be obtained from contextual signatures. For example, consider a cap removal signature that is present but that exhibits unusual repeated pressure spikes not representing enough pressure to remove the cap. Such an unusual cap removal signatures may suggest that the user is having difficulty removing the cap, for example because of limited dexterity, because the cap was poorly designed, or for some other reason. Similarly, pauses in pressure and/or orientation traces 0278 and 0274 before administering eyedrops may indicate difficulty dispensing the medication, but also may that the user is hesitating and/or resistant in taking the medication. Information regarding how a user is taking (or not taking) a medication may be of use in improving adherence, for example a health care provider who is aware of difficulty in opening the bottle may change a prescription to specify a different bottle.

In addition, with sufficiently rich trace information (e.g. high resolution in time, pressure, orientation, etc., and/or traces reflecting suitable data types for the particular question under consideration), it may be possible to identify other matters of interest. For example, how a medication bottle is opened, handled, and so forth may vary among individuals. Thus, the “open bottle” signature for a patient may be distinguishable from the signature that would be left by another person opening the same bottle, and thus it may be possible to know whether someone else has been accessing and/or taking the medication. While this may have interest as a safety matter (e.g., in helping avoid children from accessing medications not prescribed for them, in addressing possible misuse of opioids or other potentially dangerous drugs by other members of the household, etc.), determining that someone else has begun opening a medication bottle instead of the patient may be of use for other reasons. For example, it may be that a patient who is having difficulty opening the bottle is asking someone else (a spouse, adult child, etc.) for assistance. In response, a health care provider could prescribe a different bottle, or could advise the person providing assistance on matters related to treatment (e.g, it may be prudent for someone assisting with a medication to be aware of possible side effects, any unusual directions for administering the medication, etc.).

Furthermore, contextual signatures may be utilized for other than data processing. For example, a contextual signature may be used as a signal to activate, deactivate, or otherwise control some other element or device. As a more concrete example, for a medication bottle having pressure and orientation sensors, the sensors may operate at low resolution in a baseline mode. However, when a contextual signature for cap removal is identified, the sensors could be switched to a higher resolution mode. Thus, high-resolution data may be obtained during/near administration of the medication, without requiring the processing power, power consumption, etc. necessary to acquire high-resolution data continuously. Alternately, consider an eyedrop medication bottle that includes pressure and orientation sensors as previously described, but that also includes a sensor in the nozzle to determine (e.g. optically) whether a droplet has been expelled. A contextual signature for removing the cap of the bottle may be used as a trigger for activating the droplet sensor (and/or a contextual signature for replacing the cap may deactivate the droplet sensor). Thus the droplet sensor may only consume power when dispensing medication is likely, and may remain offline otherwise. Other alternatives also may be suitable.

Similarly, medication event signatures also may have functions other than data processing. For example, a device with a wireless communicator may routinely leave that communicator in sleep mode, but activate the communicator when a medication signature for administering an eyedrop is identified (e.g., so as to transmit data regarding use of the medication to some other device, person, etc.).

With regard to the aforementioned “richness” of data, it is noted that FIG. 2 is presented as a simple example of traces that may exhibit signatures. FIG. 2 does not attempt to illustrate abnormalities, distinguish among individuals, etc. in the orientation and pressure traces 0274 and 0278, nor does FIG. 2 necessarily exhibit sufficient richness to reveal such features. More detailed illustrations are presented subsequently herein.

Again with regard to signatures, it is noted that signatures need not be limited only to positive signatures, that is, signatures that indicate that medication is administered or that some contextual event is taking place. For example, a high pressure on a medication bottle not preceded by removal of the cap may be considered a negative signature, i.e. a signature that medication has not been administered.

In addition, it is not required that be definitive. That is, signatures may not in and of themselves be considered either certainly conclusive (that a medication definitely is being administered) or certainly exclusive (that the medication definitely is not being administered). Signatures may be considered as contributing to confidence, rather than definitively indicating, that some event has or has not taken place. Signatures also may be interpreted collectively, in groups of two or more, in order to determine a confidence that a medication has been (or has not been) administered. Thus, just as signatures themselves may not be based on a single data point but rather a trace, determinations of whether medication has been taken also are not necessarily based on a single signature but rather may be based on multiple signatures. For example, a signature corresponding with squeezing out a droplet, in itself and without other signatures, may be assigned a low confidence that a medication has been delivered. By contrast, signatures for lifting a bottle, shaking the bottle (e.g. to mix medication), removing the cap, tilting the bottle, and squeezing the bottle, in order and within some time range, may be assigned a high confidence. As with considering signatures themselves, evaluating signatures to determine confidence that a medication instance has been carried out may consider order, timing, other factors, etc.

Also, as with signatures, confidence for signatures may be positive or negative. Certain signatures may increase confidence that medication has been administered, while other signatures may decrease confidence. Furthermore, increase and/or decrease may be conditional; if signatures for shaking a bottle, removing a cap, and squeezing out a droplet are present, but are in the wrong order (e.g., squeezing then shaking then removing), the presence of those or other signatures may decrease confidence rather than increase confidence.

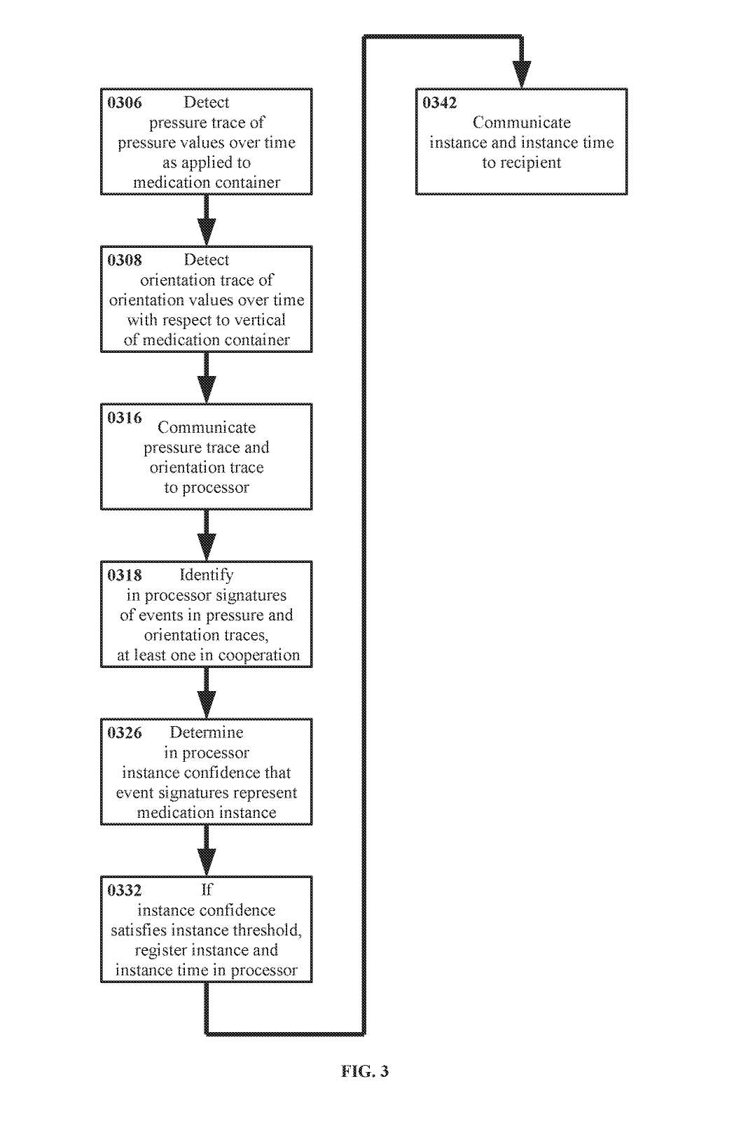

Now with reference to FIG. 3, an example method for collecting data and determining adherence is described.

In FIG. 3, a pressure trace of pressure values as applied to a medication container is detected 0306. The arrangements by which pressure traces may be determined are not limited. Typically, though not necessarily, direct pressure sensing may be used, such as by disposing sensors and/or probes for detecting squeezing forces applied to the container, deformation of the container wall, etc., on and/or in the container. For example, sensors/probes may be disposed internally within the container, embedded into the wall of the container, incorporated into a layer disposed on the wall such as a label, etc. As a more concrete example, a piezo probe or a capacitive/resistive probe may be disposed within an adhesive label that is adhered to the outer surface of a medication bottle, with data being sensed from those probes to determine the forces applied to the container over time. However, other arrangements, including but not limited to optical detection (e.g., a camera that detects deformation of the container, potentially from some distance away and not necessarily physically engaged with the bottle), may be equally suitable.

An orientation trace of orientation values for the medication container with respect to the vertical is also detected 0308. The arrangements by which orientation traces may be determined also are not limited. Typically, though not necessarily, sensors and/or probes engaged with the container may measure orientation, change in orientation, etc. For example, a gyroscope may be affixed to the container in some manner, such as being connected to the base thereof, embedded into the container wall, etc., such that the gyroscope rotates with the container and rotation of the container thus may be determined from rotation of the gyroscope. However, other arrangements, including but not limited to optical detection (e.g., a camera that detects the orientation of the container), may be equally suitable.

It is noted that although certain examples of traces (e.g. 0274 and 0278 in FIG. 2) are shown as graphical plots, this is presented for illustrative purposes and is not limiting. While plotting traces graphically is not prohibited, neither is such required. In certain embodiments, traces may be entirely numerical, e.g. a list of values for pressure, orientation, etc.

Returning to FIG. 3, the pressure trace and orientation trace are communicated 0316 to a processor. Typically, though not necessarily, the processor may be physically engaged with the container, with communication being carried out through conductive leads, etc. In particular, it is noted that for embodiments wherein the processor (and likewise the sensors, and/or portions thereof) is physically engaged with the container, the processor (and/or sensors) may be removably engaged. For example, the processor, sensors, etc. may be disposed in a “snap-on” fixture that attaches temporarily with the remainder of the medication container. However other arrangements, such as a processor that is some distance from the container, and/or communication through wireless means, also may be suitable. Neither the processor, the processor's position, nor the manner of communication is limited.

In the processor, one or more signatures are identified 0318 in the traces, with each signature being consistent with some corresponding event associated with an instance of administering a medication to a subject. For example, as previously noted signatures may correspond with removing a cap from a bottle, squeezing the bottle to extrude a droplet, etc. Typically, though not necessarily, the signatures are identified 0318 through execution of instructions instantiated on the processor and/or reference to data instantiated thereon. For example, standards may be defined within the processor for various signatures, for example specifying the necessary height, width, shape, etc. of a pressure trace peak for that peak to be interpreted as corresponding with a user squeezing the bottle to extrude a droplet. In such case, peaks within the pressure trace (as communicated in step 0316) may be compared in the processor against such standards. However, other arrangements also may be suitable.

It is noted that not all features within a trace, nor the entirety of a trace itself, are required to form or be considered as signatures. Peaks (for example) may be present within a data trace that do not correspond to any event of interest. While presumably a variation in pressure value and/or other data may have some cause (even if only the noise inherent in electronic circuits), and that cause may correspond to some real-world event, not all such variations will be of interest to all embodiments. For example, if the bottle is in a pocket and is nudged by another object in that pocket, producing a slight peak, this peak may not be either defined as or identified as a signature, and the corresponding event (the nudge) may not be considered for any purpose. Likewise, portions of a trace may be of no consequence for a given embodiment, and may not be identified as signatures or otherwise considered.

Still with reference to FIG. 3, in identifying 0318 signatures in the traces, at least one such signature is identified 0318 through consideration of the pressure trace and the orientation trace in cooperation. For example, as previously described with regard to FIG. 2, a signature for “squeezing out a droplet” may be defined with regard to features in both the orientation trace (e.g., the container is inclined to some position or range of positions) and the pressure trace (e.g., some minimum pressure is applied to the bottle). It is noted that consideration of two traces does not require, and is not limited to, consideration of two traces at the same moment in time. To continue the example of a “squeezing out a droplet” signature, it may be required that pressure on the container exceeds a minimum value at some time, while the container underwent a change in orientation into an inclined position at some earlier time. In such case, the change in orientation and the applied pressure are not simultaneous (i.e., the bottle is tipped first, and then squeezed); nevertheless such non-simultaneous data features in the traces may be considered as a single signature, and identifying that signature would constitute identifying a signature with the pressure and orientation traces in cooperation.

Not all signatures necessarily will be identified through the cooperation of two or more traces; some signatures may be identified with only a single trace, or (in embodiments with three or more data traces) with more than two traces. However, although it is not required that all signatures consider multiple traces in cooperation, neither is such prohibited.

Continuing in FIG. 3, a determination is made 0326 in the processor as to a medication instance confidence, that is, the degree to which any signatures as may be present collectively indicate that medication has been administered. Typically though not necessarily, the confidence is determined 0326 through execution of instructions instantiated on the processor and/or reference to data instantiated thereon. For example, the presence or absence of certain signatures, the degree to which the signatures meet definitions therefor, the order of signatures, the timing between or among signatures, etc. may be considered to determine some confidence level as to whether the traces indicate that the medication has been administered. Confidence may be determined and/or expressed in a variety of manners. For example, confidence may be a simple yes/no, i.e., the medication was administered or was not. However, confidence need not be, and need not include, a definitive yes/no. For example, confidence may include a percentage or similar value, i.e. 0.90 confidence, 82.3% confidence, etc. Confidence also may be expressed more abstractly, for example “level one”, “level two”, or “level three”. Other arrangements also may be suitable.

It is noted that certain embodiments may first determine one or more signature confidences, then determine instance confidences therefrom. For example, each signature may be evaluated as to the likelihood that the trace therefor is indeed a valid representation of a medication event (a “signature validity confidence”), and/or each signature may be assigned some weighting with regard to how much that signature contributes to the confidence that overall the traces reflect a medication instance (a “signature significance confidence”). However, while such arrangements are not prohibited, neither are such arrangements required; instance confidence may be determined without first determining confidence(s) for signatures.

Still with reference to FIG. 3, if the instance confidence is determined to meet an instance threshold therefor, an instance of medication administration and the instance time for that medication instance is registered 0332 in the processor. In colloquial terms, if the signatures show that the medication has been taken, the determination that medication has been taken and the time at which the medication has been determined to have been taken are recorded in some fashion. How the information is registered 0332 is not limited. The information may be flagged for transmission to some other entity (a database, another processor, a human monitor, etc.), may be flagged for storage in a data store (such as a hard drive, solid state drive, etc.), or otherwise noted in some fashion. In addition, the precise information that may be registered 0332 is not otherwise limited; information may be as minimal as a time (with the fact of the instance being implicit in the time), but may also include other information such as the instance confidence, the relevant signatures, some or all of the traces, identifying information such as a processor ID, patient name or reference number, the type/dose of medication, etc.

In addition, it is noted that for certain embodiments the confidence threshold may be zero, or near-zero. That is, medication instances may be logged along with times therefor, even if confidence is minimal. For example, this may be useful in situations where data collection is of particular importance, such as during a trial period for a patient when a learning algorithm is still becoming accustomed to the particular patient, or during an early system test for a medication trial where learning to avoid false negatives is worth processing large amounts of data. In such instance, it may be useful to collect a large number of real, potential, or even false medication instances, so as to form a baseline, provide data for distinguishing real medication instances from false positives, and so forth.

Furthermore, although the medication instance time may ideally be a precise time at which the medication is administered, exactness is not necessarily required for all embodiments. In certain cases, the moments at which a medication is administered may be clearly and definitively identified (such as at times 8 and 11 in the traces shown in FIG. 2), but this may not always be true. Notably, certain medications may not be taken instantaneously, e.g. a skin cream that must be applied over a large area of the body may take several minutes to apply, so it may not be possible to define (much less measure) the time at which the medication is applied “to the second”. Approximations may be suitable, e.g. rounding to the nearest minute, using the start time as the instance time, or even using the time at which the instance is identified in the processor as the instance time. Other arrangements also may be suitable.

Continuing in FIG. 3, the medication instance and medication instance time are communicated 0342 to a recipient. As previously noted, the medication instance and medication instance time as registered 0332 may then be stored, transmitted, etc. Such storage, transmission, etc. may fulfill step 0342; that is, for certain embodiments the recipient may be a hard drive or database. However, the recipient may also be a living person, such as the subject himself/herself; for example the instance and time may be displayed on a readout (e.g., on the container itself), announced through a speaker, etc. so as to confirm to the subject that their use of the medication has been confirmed. Alternately, the information may be directed to (for example) a smart phone in possession of the subject, or some person caring for the subject (a parent, spouse, medical professional, etc.). Other arrangements also may be suitable.

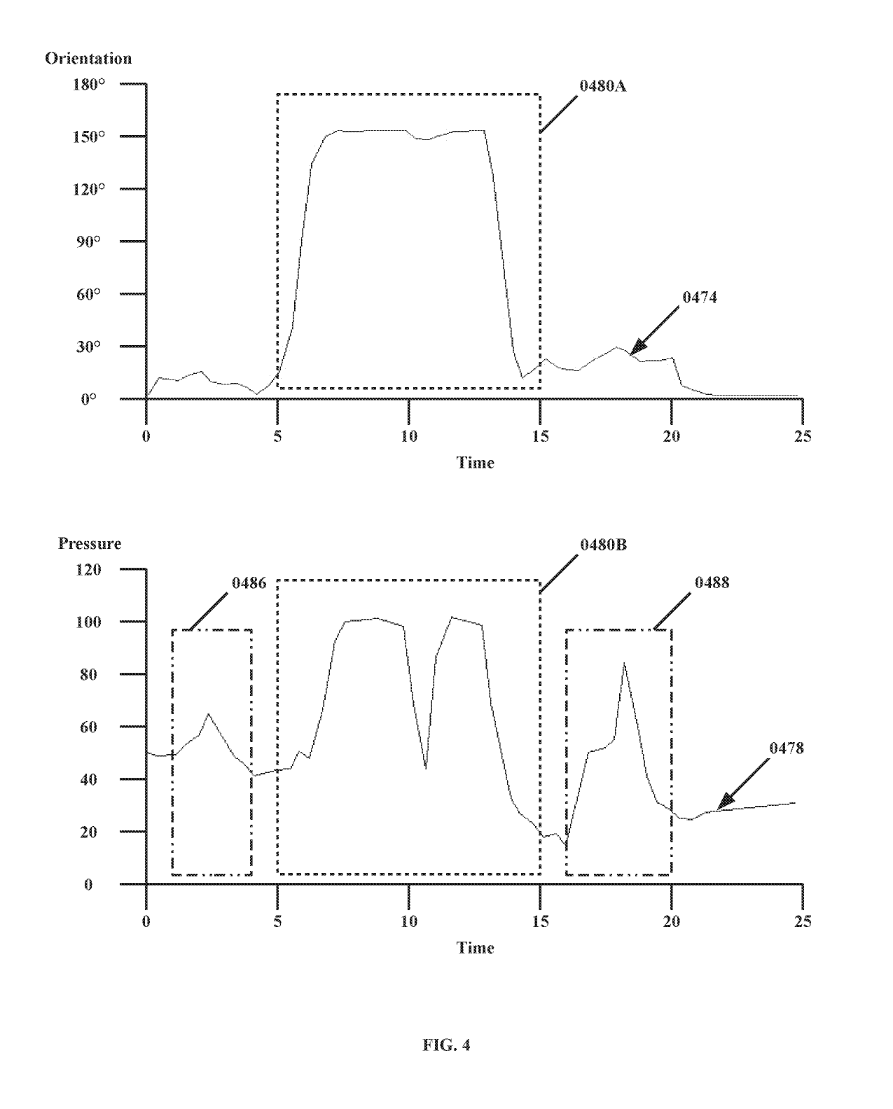

Now with reference collectively to FIG. 4 through FIG. 10, as has been noted previously it may be useful in certain embodiments to have data and/or traces that are “rich” in information. FIG. 2 previously illustrated data traces with a very simple configuration for purposes of clarity (simple rises and falls, uniform slopes, identical signatures for the same event, etc.), but in practice features in data traces may be more complex, less uniform, etc. For example, traces reflecting droplet events may differ from traces reflecting cap opening events, traces for two droplet events may differ even when performed consecutively with the same medication and by the same person, traces for the a given type of event (e.g. squeezing out a droplet) may vary with different medications and/or people, etc. Thus, while rich data may appear “messier”, obtaining and considering rich data also may provide advantages.

With reference specifically to FIG. 4, traces 0474 and 0478 for orientation and pressure for an eyedrop medication bottle are plotted graphically. The system under consideration is thus at least somewhat similar to that of FIG. 2 (and by extension to events shown in FIG. 1). In addition, the overall configuration of the traces 0474 and 0478 may resemble the configuration of traces 0274 and 0278 in FIG. 2, as may reflect similar events (e.g., removing a medication bottle cap, administering two droplets of medication, and replacing the bottle cap).

In particular, in FIG. 4 the pressure trace 0478 exhibits a peak approximately midway between times 0 and 5, and another such peak between times 15 and 20, while the orientation trace 0474 is comparatively stable at those times. Also, the pressure trace 0478 exhibits two consecutive peaks just before and after time 10, while the orientation trace 0474 exhibits a high plateau from approximately time 5 to time 15. Comparison with FIG. 2 may reveal similar overall features.

For clarity, FIG. 4 also explicitly identifies portions of traces 0474 and 0478 as exhibiting signatures 0480A, 0480B, 0486, and 0488. More particularly, the plateau in the orientation trace 0474 and the paired spikes in the pressure trace 0478 are identified as signature elements 0480A and 0480B respectively, together forming droplet signature 0480 as may be consistent with squeezing droplets of eye medication out of a bottle into a subject's eyes. Likewise, the preceding and following spikes in pressure trace 0478 are identified as cap opening and cap closing signatures 0486 and 0488 respectively, as may be consistent with removing/opening a medication bottle cap and replacing/closing the cap, respectively. Such an arrangement again may at least somewhat resemble that of FIG. 2. However, where FIG. 2 presented a deliberately “pristine” arrangement for purposes of clarity, FIG. 4 presents data that may be less clear-cut. (It is noted that such “messiness” in the traces 0474 and 0478 may more realistically reflect data obtained from a real-world system in practice.)

For example, as may be seen the paired peaks in the pressure trace 0478 (also identified as signature element 0480B) exhibit not merely simple ups and downs, but variable slopes, flattened tops, etc.; that is, the pressure trace 0478 therein has sufficient resolution and richness that relatively complex shapes may be recognized therein (e.g., as opposed to simple “step up, step down” data). In addition, as may be seen in the pressure trace 0478 the first and second peaks therein (marked together as droplet signature element 0480B) are not identical. This is so even though presumably the same person has squeezed the same bottle with the same medication twice consecutively, within a matter of a few seconds. A certain degree of variation may be expected in “real-life” data, e.g., a subject may not squeeze with exactly consistent pressure, increase/decrease pressure at exactly consistent rates, hold pressure for exactly consistent durations, etc. To accommodate such factors, definitions for various signatures may include some degree of breadth rather than specifying a highly particular curve shape, etc. In addition, signatures may be defined so as to vary over time, e.g., to accommodate changes in the necessary pressure as a bottle is gradually emptied over many uses. Signatures also may be adaptive, such that features identified as signatures modify definitions for future signatures, e.g., through the use of machine-learning algorithms. As a consequence, signatures may be (and/or may become over time) customized to specific users; two users of the same medication with the same bottle design may develop different droplet signatures, for example.

Thus, richness of data such as shown in traces 0474 and 0478 of FIG. 4 may enable different features to be distinguished from one another. Consequently, with suitable data traces and/or analysis thereof, signatures of different events may be distinguished from one another (thus at least potentially enabling consideration of contextual signatures, if such contextual signatures may be distinguished from medication signatures), signatures for different users may be distinguished from one another, false positives may be distinguished from signatures for real events, etc. Other information also may be determined therefrom.

For example, with regard to distinguishing signatures of different events, even ignoring the orientation trace 0474, examination of the pressure trace 0478 alone reveals visible differences between the peaks in the droplet signature element 0480B and the peaks in the cap opening and cap closing signatures 0486 and 0488. The peaks for the cap opening and cap closing signatures 0486 and 0488 are more pointed than the peaks for the droplet signature element 0480B, for instance, and other visible differences also may be observed. Coupling such distinctions with the consideration of the orientation trace 0474 in cooperation with the pressure trace 0478 also may be useful as previously noted (e.g., for a “real” droplet signature the bottle may be expected to be inclined, thus a signature of a real droplet event may be expected to exhibit the visible plateau in droplet signature element 0480A).

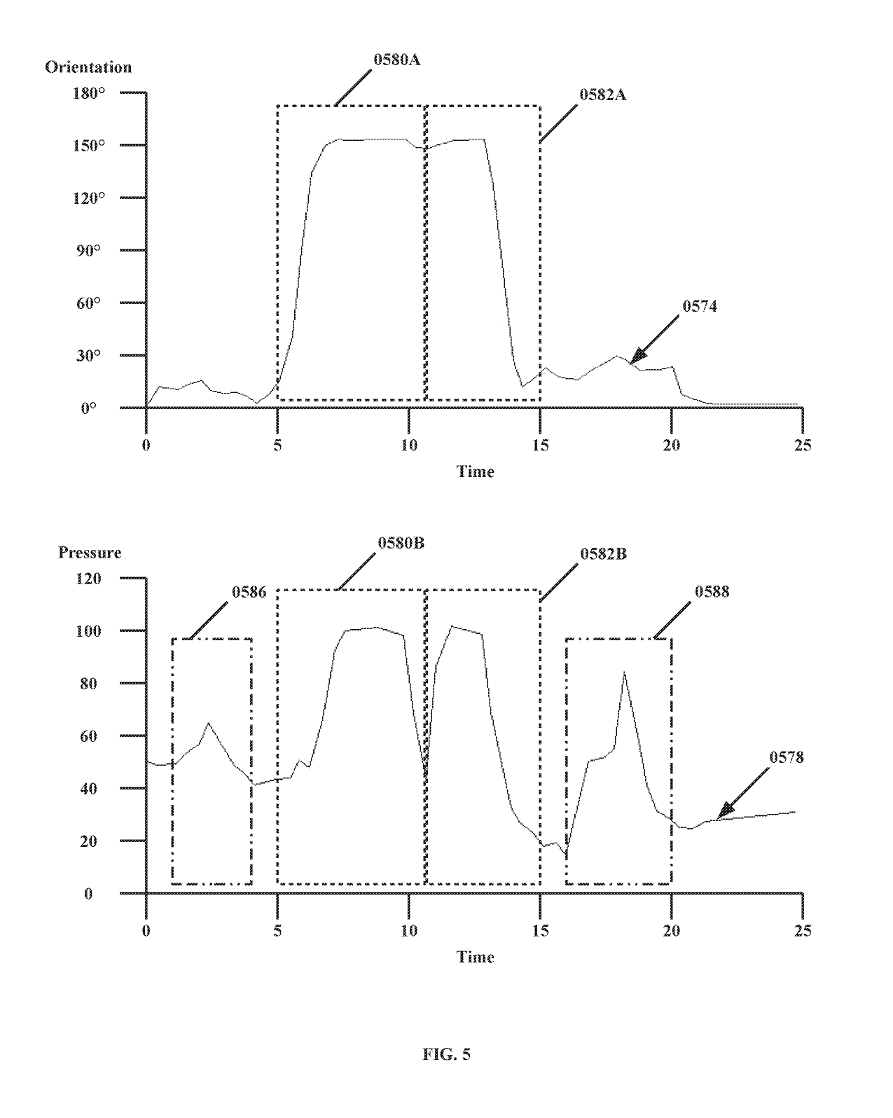

Now with reference to FIG. 5, orientation and pressure traces 0574 and 0578 again are shown, it is noted that signatures, features in traces that may or may not be signatures, definitions for signatures, analysis for signatures, etc., may vary considerably. While not necessarily arbitrary, varying portions (times, durations, etc.) of traces may be considered, features may be addressed as varying numbers of potential signatures, and so forth. For example, orientation and pressure traces 0574 and 0578 in FIG. 5 appear very similar to FIG. 4, however where FIG. 4 addressed a single droplet signature 0480A and 0480B encompassing two peaks (both droplets), FIG. 5 addresses each peak individually as first droplet signature 0580A and 0580B and second droplet signature 0582A and 0582B. Either approach may be suitable; signatures may define so as to address a single event, multiple events, part of an event, etc. (FIG. 5 also shows cap opening and cap closing signatures 0586 and 0588, similar to FIG. 4.)

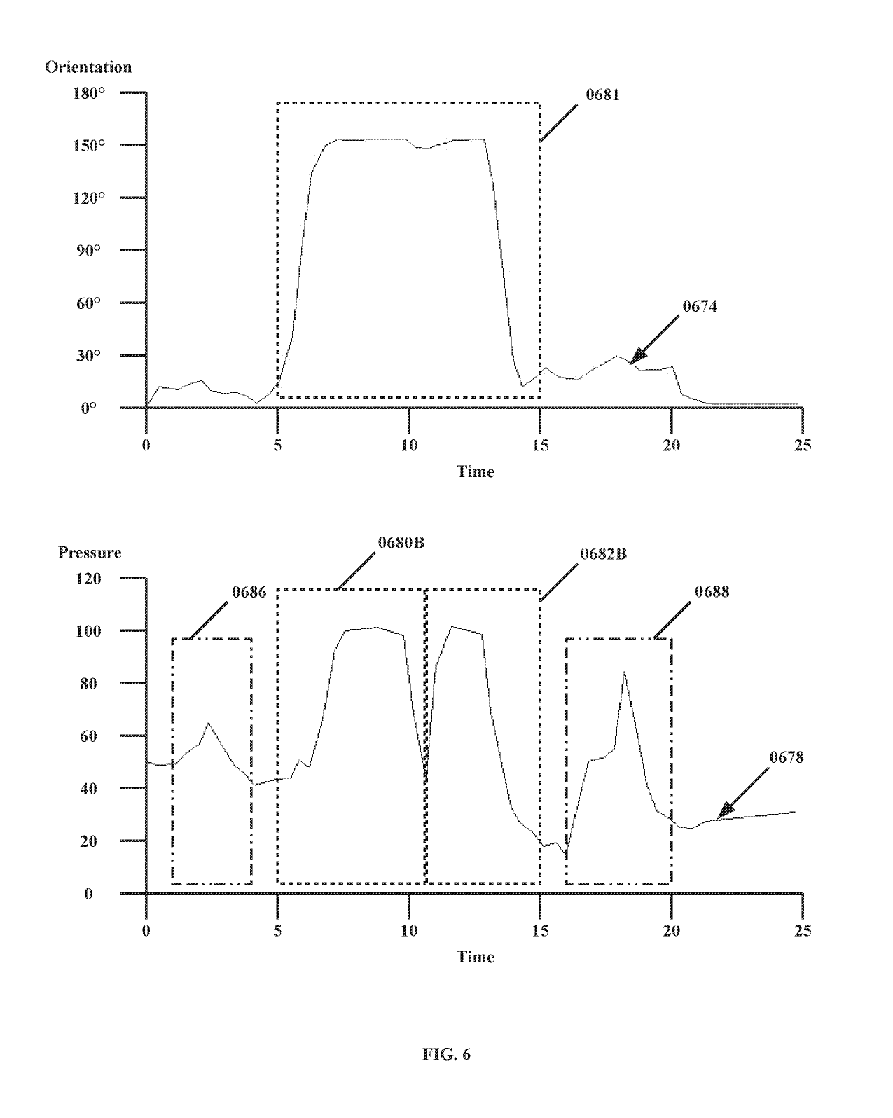

Turning to FIG. 6, it is noted also that signatures may be defined differently with respect to different traces. FIG. 6 shows orientation and pressure traces 0674 and 0678 along with cap removal and cap replacement signatures 0686 and 0688. Like FIG. 5, in FIG. 6 the two droplet peaks are addressed separately as first droplet element signature 0680B and second droplet element signature 0682B in the pressure trace 0678, however in the orientation trace 0674 only a single droplet element signature 0681 is identified. As may be seen, signatures addressing two (or more) traces in cooperation need not define the same number of signatures, even when covering the same overall time.

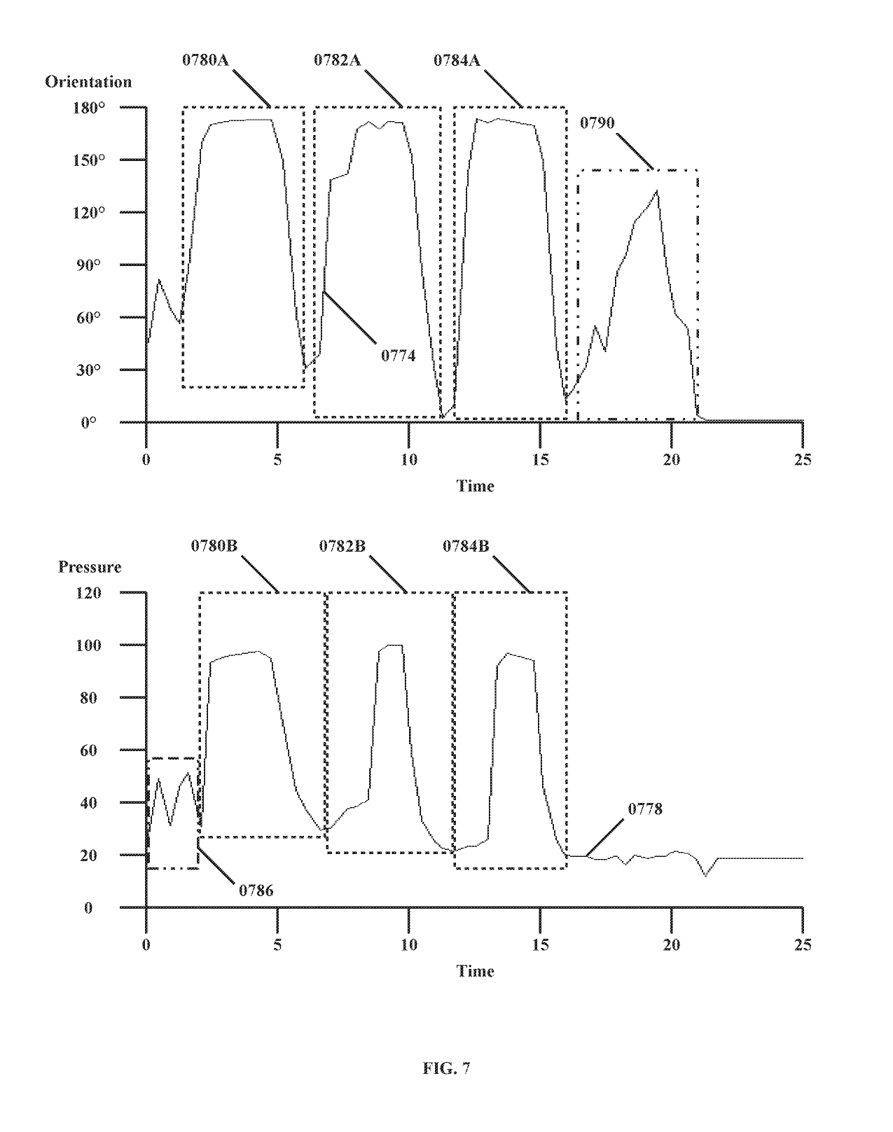

Now with reference to FIG. 7, as indicated previously a rich trace may facilitate determining not just the fact of a medication instance (i.e., that a subject has taken the medication) but at least potentially also may identify other information regarding how a medication is being taken. For example, typically a medication regimen may specify a dose, e.g., two droplets per eye (as shown in FIG. 4 through FIG. 6). As may be seen in FIG. 7, orientation and pressure traces 0774 and 0778 do not appear to correspond with a dosage of two droplets. The orientation trace 0774 exhibits three consecutive approximately flat-topped peaks, identified as first droplet signature element 0780A, second droplet signature element 0782A, and third droplet signature element 0784A; the pressure trace 0778 exhibits three flat-topped peaks approximately aligned in time with the orientation trace 0774, identified as first droplet signature element 0780B, second droplet signature element 0782B, and third droplet signature element 0784B. Thus, it may be inferred from FIG. 7 that three drops of medication have been dispensed; if the regimen for the medication specified two drops, the arrangement in FIG. 7 may reflect a deviation from the prescribed dosage. Typically, dosage may be significant in determining the effect of medications (whether for research or for treatment). Incorrect dosage is estimated to be a common deviation from adherence, whether by error (patients accidentally squeeze too many drops, etc.) or by design (patients deliberately take more medication than prescribed, e.g., under the assumption that “more is better”, etc.); however, the actual frequency and degree of incorrect dosage may be unclear, as dosage (like taking a medication at all) conventionally may be self-reported, with issues already noted above.

Other potential anomalies also may be observed in FIG. 7. A double peak in the pressure trace 0778 is visible just after time 0, and is identified as being a signature 0786 of a bottle cap being removed. However, no corresponding signature is visible for replacing the cap later. Whether the lack of a cap replacement signature is due to a bad sensor, noise, or the patient failing to replace the cap, the lack of such a signature may be useful. If an anomaly is not known to exist, addressing whatever is causing the anomaly may be severely problematic. Thus the availability of a trace 0778 that exhibits the anomaly may enable a response to the anomaly, regardless of cause. For example, if a hardware problem exists (a bad sensor, etc.) the hardware may be repaired or replaced, if the patient is inadvertently leaving the medication bottle open a reminder may be provided, and so forth.

In addition, a strong peak in the orientation trace 0774 is visible beginning after time 15, and is identified in FIG. 7 as unspecified signature 0790. If no corresponding event is anticipated as part of dispensing the medication, it may not be clear what unspecified signature 0790 is a signature of, or if the spike is relevant at all. In practice it is not uncommon to tilt a medication container briefly in such manner while setting the container down, and in this particular instance the spike referred to as unspecified signature 0790 likely does not relate to adherence or nonadherence at all. However, as noted above the simple availability of such information from the orientation trace 0774 (and potentially from the pressure trace 0778 as well) may at least enable the possibility of identifying relevant information therefrom (whether or not such information is actionable).

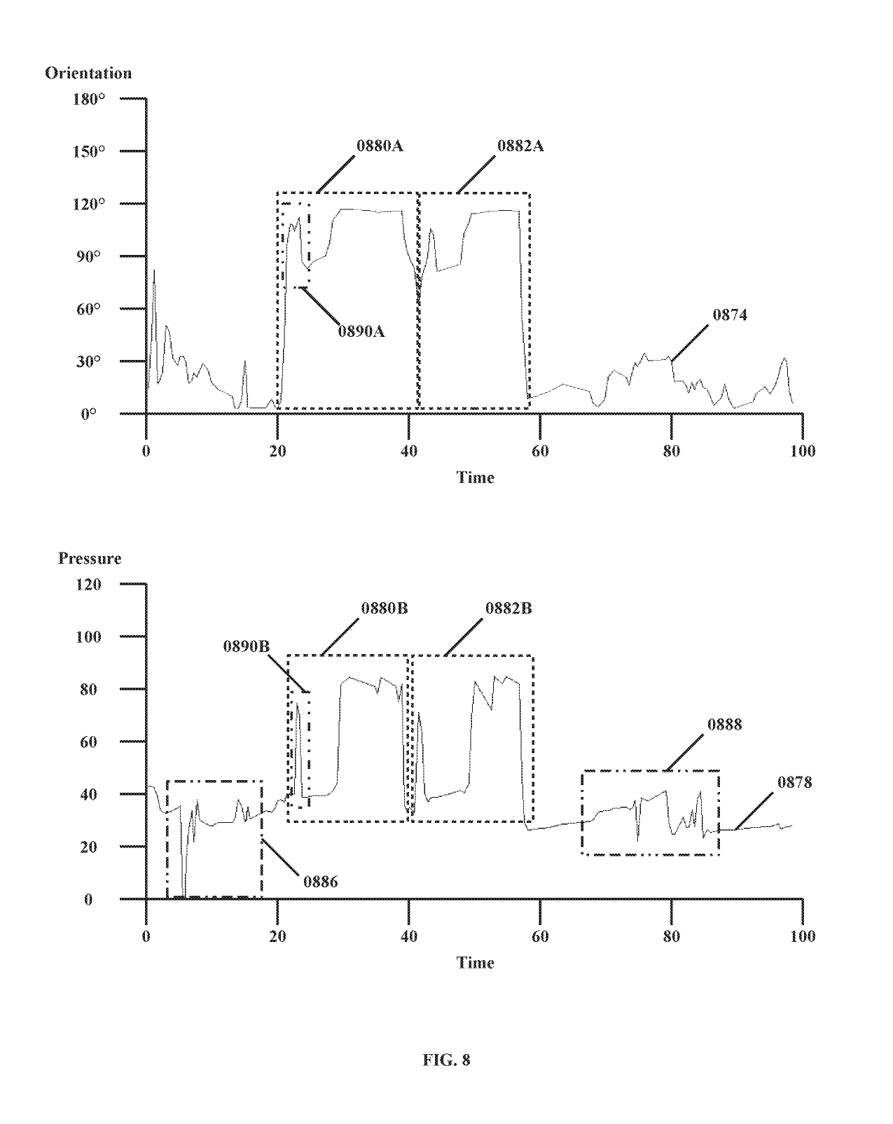

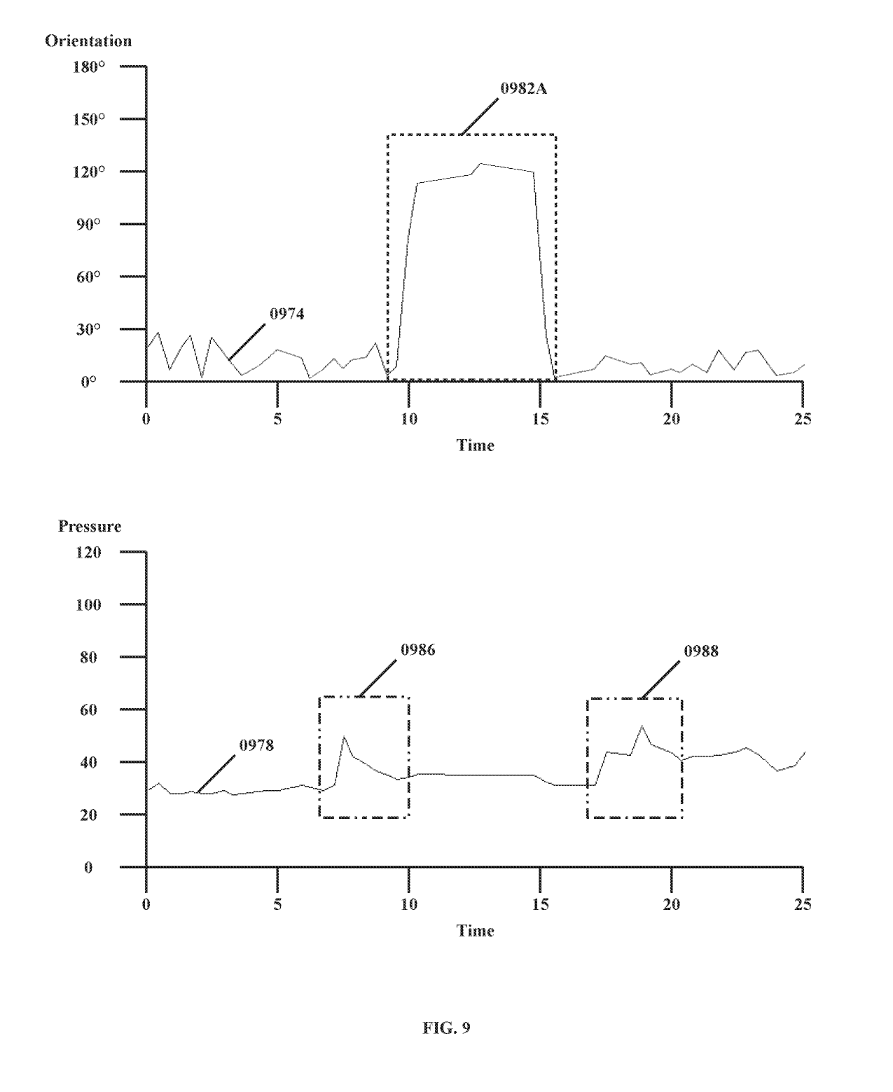

Moving on to FIG. 8, another example of information besides a medication event itself is shown. FIG. 8 shows features in orientation and pressure traces 0874 and 0878 identified as a cap removal signature 0886, a first droplet signature 0880A and 0880B, a second droplet signature 0882A and 0882B, and a cap replacement signature 0888. However, as may be observed the orientation and pressure traces 0874 and 0878 both exhibit various sharp rises and falls, which do not have readily visible counterparts in FIG. 4 through FIG. 7. One such feature is identified at unspecified signature 0890A and 0890B. Several observations may be made.

First, the anomalies appear in both the orientation trace 0874 and the pressure trace 0878. It may be judged unlikely that two sensors would both fail in such a similar manner. Certain features (e.g. the leading spike visible in 0890A/0890B) also appear in both droplet signatures 0880A/0880B and 0882A/0882B, which may be taken to indicate that random noise is unlikely. Further, the leading spike in 0890A/0890B is visible in both the orientation trace 0874 and the pressure trace 0878 at approximately the same time. Taken together, these may suggest that the anomalous variations visible in FIG. 8 reflect real-world phenomena. In fact, the orientation and pressure traces 0874 and 0878 in FIG. 8 correspond at least approximately to sensor data for a person with shaky hands, who may be struggling to open a medication bottle, administer drops, and close the bottle again. If true, this may have direct implications for use of the medication, e.g., does the shaking suggest that droplets may be missing the patient's eyes altogether? A health care provider may for example ask the patient about this (and at least potentially analysis of the traces 0874 and 0878, and/or traces from other sensors, may reveal such information). Somewhat less directly, if the traces 0874 and 0878 indicate severe shaking to the point that adherence may be compromised, it may be useful to modify the medication regimen in some fashion, e.g., by specifying that the medication be dispensed in an easy-to-open bottle, etc. Likewise, if shaking appears over time after the patient begins using the medication, it may be that shaking could be a side effect of the medication; such information may then be obtained without necessarily relying only on patient self-reporting. In addition, while perhaps incidental to the particular medication being used in FIG. 8, the existence, prevalence, and severity of shaking may be useful diagnostically for the patient. For example, shaking hands may be a symptom of some other condition, one that also could be addressed medically or otherwise. Identifying such symptoms (or otherwise determining the possibility of relevant medical issues) could be of particular interest if likely medical conditions are contraindications for the medication in question, or are suggestive of medical concerns that are suspected to be related to the reason the patient is taking the medication in the first place (e.g., being a sign that a disease is progressing).

In addition, the “messy” traces 0874 and 0878 in FIG. 8 also may serve as an example of another potential advantage of rich data traces: namely, rich data traces may help to accommodate less-than-ideal inputs and/or circumstances.

As may be seen (and as already noted), traces 0874 and 0878 exhibit numerous sharp rises and falls. Those rises and falls may be anomalous to the larger events under consideration; that is, the rises and falls visible in FIG. 8 may not in themselves define or help define signatures for events of interest, such as taking the medication, removing the container cap, etc. As noted previously, the rises and falls visible in FIG. 8 may be indicative of shaking in the subject's hands, however other sources may produce anomalies, such as electronic noise in a circuit, fumbling with a difficult-to-open cap on medication bottle, etc. Likewise, other factors may cause data traces to deviate from an idealized form, such as miscalibrated or malfunctioning sensors, loss of data in transmission, varying patient behavior (e.g., different patients may tilt an eyedrop bottle to different inclinations), and so forth. Regardless of source, in practice data traces may exhibit less-than-ideal forms.

Furthermore, FIG. 8 may illuminate certain matters regarding confidence determinations, as mentioned previously with respect to step 0332 in FIG. 3. With simple data, evaluation of features may be limited to simple conclusions, e.g., a binary determination that a signature is present or is not present. For example, referring to FIG. 2, traces therein are such that peaks may not be distinct from one another. If the data available is simple, then it may be that only simple evaluations can be performed with that data. For example, if all pressure peaks look alike, then only simple conclusions may be drawn, i.e., a pressure peak either is a signature of some event or is not. The presence of anomalies in such simple data may result in features being rejected as signatures, when in fact at least some such trace features may represent real events. Conversely, anomalies may be incorrectly identified as signatures of real events. In other words, a combination of information-poor data traces and less-than-ideal operating circumstances may contribute to false negatives and/or false positives. To refer again to FIG. 8, an anomalous pressure peak—such as the feature identified as 0890B—could be interpreted as representing a real squeeze to a bottle, while an anomalous dip—such as the drop in the pressure trace 0878 immediately after 0890B—could mask a real squeeze. This may be particularly true for single point data, e.g. “does the pressure exceed a given minimum at any point?”

However, as is visible in FIG. 8 features such as droplet signatures 880A/880B and 882A/882B and cap removal and replacement signatures 0886 and 0888 may remain discernible, even though the traces 0874 and 0878 exhibit numerous anomalies. Richness of trace data may facilitate identifying real signatures and/or rejecting false signatures even with less-than-ideal data.

In addition, it may be useful to address the potential for less-than-ideal data through considering signatures and/or traces as a whole (not merely in FIG. 8 but in general) with greater subtlety than a simple binary “yes/no” evaluation. While in certain embodiments it may be suitable to simply interpret trace features as binary, i.e., “this feature is a signature” vs. “this feature is not”, in other embodiments it may be suitable to use non-binary approaches. For instance, as noted the pressure trace 0878 exhibits an anomalous spike 0890B, which may contribute to an atypical overall form for the first droplet signature element 0880B. Rather than attempting to make a definitive yes/no determination as to whether the relevant portion of the pressure trace 0878 is or is not a signature for a squeeze of a medication bottle, it may be useful to consider that the pressure trace 0878 may not be definitive. For example, some confidence level may be assigned, based on the degree to which the pressure trace 0878 matches a definition for a droplet squeeze signature. Thus, features may be identified not merely as being a signature of an event or not, but as being a signature with 85% confidence, with low/moderate/high confidence, as red/orange/yellow/green confidence, or otherwise as indicating varying degrees of confidence. In more colloquial terms, trace features may be evaluated not merely in black-and-white, but in shades of gray.

It is noted that to at least some degree, a viable determination of confidence may depend on the availability of rich trace data. For example, if features cannot be distinguished from one another—if peaks “all look the same”—then determinations may be similarly limited. Thus, in at least certain embodiments such confidence determinations may derive at least in part from determination of traces, and in particular traces sufficiently rich as to enable distinguishing features from one another, as previously described.

Confidence may be applied to individual signatures, to parts of individual signatures, to groups of signatures, etc., and/or in combination. For example, each individual feature may be assigned a confidence (e.g., as a percentage, though other arrangements may be suitable). Alternately, the up-slope, peak, and down-slope of a single rise-and-fall in a trace may each be assigned individual sub-confidences; those sub-confidences may be used to determine a confidence for the signature as a whole. As yet another alternative, confidences for various signatures may be combined to determine an overall confidence for a medication event. For example, if a bottle opening signature, two droplet signatures, and a bottle closing signature are all observed with some degree of confidence, then those confidences (and/or other factors, such as whether the potential signatures appear in an expected order) may be combined to determine an overall confidence as to whether an instance of a medication being taken has occurred. However, it may not be necessary for all embodiments to determine overall medication instance confidence from individual signature confidences; determining medication instance confidence without determining signature confidences may be equally suitable.

The manner by which confidence may be determined (if determined at all) is not limited. Typically, though not necessarily, confidence determination may be dependent at least in part on the particulars of how a signature is defined, and how traces are compared to such a definition to determine whether a signature is present. For example, if signatures are defined geometrically and traces are analyzed geometrically to determine whether a feature in a trace “fits” the geometry of a signature, then confidence may be determined based on how well that feature fits that defined signature geometry. Other arrangements, including but not limited to non-geometric mathematical analyses, also may be suitable.

While confidence is described initially with regard to the example of FIG. 8, it is emphasized that confidence determinations are not limited only to circumstances similar to those in FIG. 8. Confidence may be considered with regard to many embodiments, and the use of confidence is not limited.

Moving on to FIG. 9, again certain examples of information besides a medication event itself as may be determined through various embodiments is shown. FIG. 9 shows orientation and pressure traces 0974 and 0978, and features therein identified as a cap removal signature 0986 and a cap replacement signature 0988. However, in FIG. 9 what may be anomalous is what is not visible. While a droplet signature element 0982A may be seen in the orientation trace 0974 (suggesting that the medication bottle has been inclined for dispensing droplets), no corresponding signature element is present in the pressure trace 0978 (as might suggest that pressure had been applied to the bottle to squeeze out droplets).

Such an arrangement may suggest several possibilities. A sensor or other system may have failed, but the presence of peaks in the cap removal signature 0986 and cap replacement signature 0988 may at least suggest the pressure sensor is functioning. Similarly, while the patient may be unable to apply pressure to expel droplets the peaks in the cap removal and replacement signatures 0986 and 0988 may suggest that the patient can apply at least some pressure; no pressure peaks (even inadequate peaks) are visible that would suggest a failed attempt to expel droplets. As another option, the patient may not be using the bottle correctly. (This may be a notable problem for inexperienced users. Someone who has never seen much less used an eyedrop bottle may simply be unaware of what to do, if not given adequate instruction. As noted elsewhere, such issues may also apply to other medical containers and/or devices, e.g., an inhaler, a hypodermic injector, etc., as embodiments are not limited to only eyedrops, to medication containers, or even necessarily only to medical applications.) As still another option, it may be that the patient is willfully pretending to use a medication without actually doing so. Patients may resist taking medications that have unpleasant side effects, for example; an example could be a child attempting to convince a parent that they are taking a medication while not doing so. Alternately, outright fraud may not be entirely unknown; individuals have been identified as “participating” in clinical trials in exchange for compensation, while deliberately not taking the medication. While presumably deceptive practices may impact whatever research is being conducted—for example reducing the apparent effectiveness of a medication because a subject is recorded as taking a medication when they are not—reliable data regarding the prevalence of such activities may not be readily available (self-reporting of fraud may not be relied upon, and accurately determining levels of fraud not actually detected may be challenging).

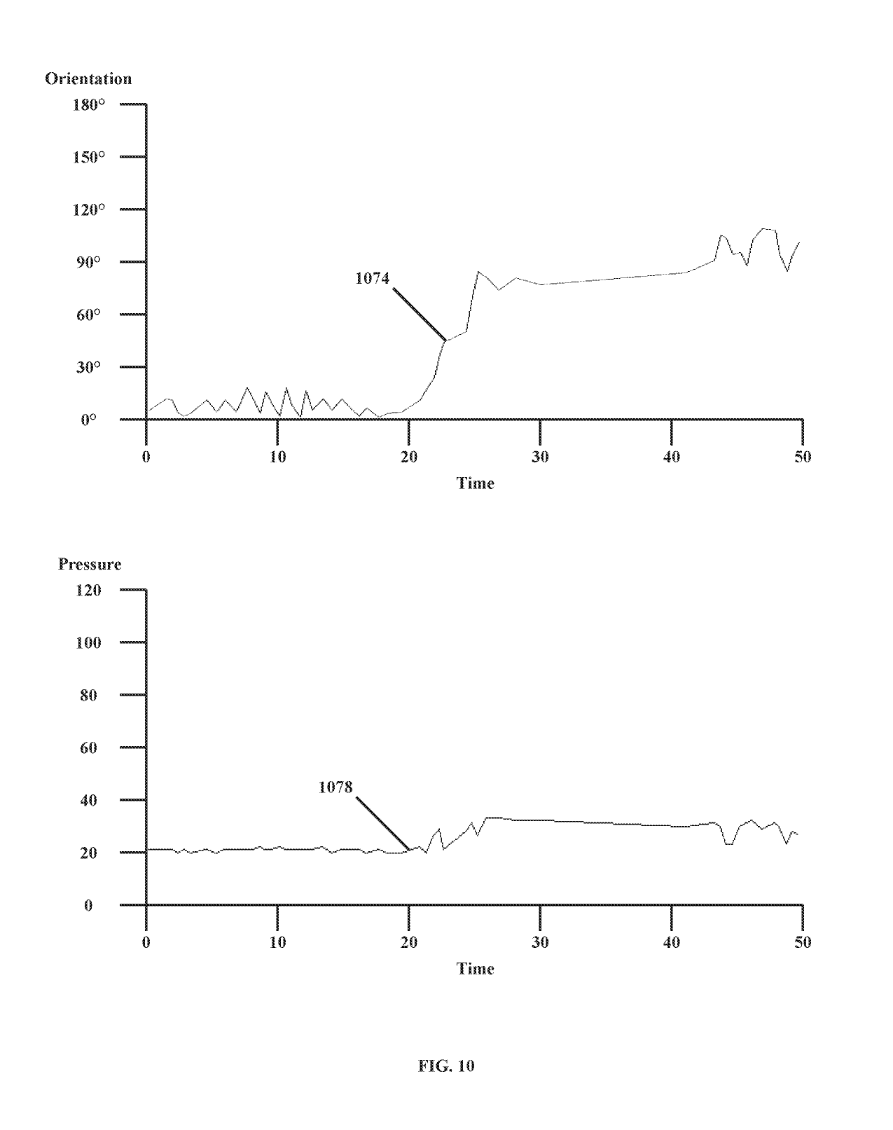

Turning to FIG. 10, additional examples of information not limited to a medication instance only are presented therein. Certain previous examples have focused on immediate context surrounding a medication event, e.g., shaking the medication opening the bottle, taking the medication, etc. However, broader context and/or apparently unrelated information also may be usefully considered.

In FIG. 10, orientation and pressure traces 1074 and 1078 are shown. As may be observed, the orientation trace 1074 exhibits a series of low (less than 30°) periodic changes in orientation from time 0 to approximately time 20. Thereafter the orientation rises to approximately 80° and then levels off. Meanwhile, the pressure trace 1078 is approximately level from time 0 to time 20, rises slightly, and then again levels off.

No signatures of events directly associated with taking a medication are marked in FIG. 10. In fact, the traces 1074 and 1078 are consistent not with taking a medication (as in certain previous examples), but rather with a person carrying a medication bottle in a pocket or bag while walking from time 0 to approximately time 20, and then sitting down beginning around time 20.

It is noted again with respect to richness of data traces that such richness may enable distinguishing not only medication-related events but also other events. It may be assumed that a person carrying a medication will spend most of their time not taking the medication. Thus, the ability to discriminate between medication-related events and whatever else the subject may do for the remainder of their day may prove useful in avoiding false positives (without placing special restrictions on patients, e.g., “only store medication in a vertical position at all times” or “do not carry medication on one's person”).

In the example of FIG. 10, when and whether a patient walks and sits may be determined with at least some degree of confidence from data traces 1074 and 1078. (It is noted that confidence as described previously with regard to FIG. 8 also may be applied to events and/or signatures that may not be directly related to medication, such as walking, sitting, etc.) While walking and sitting may not be considered part of taking a medication per se, nevertheless such information may have bearing on whether a medication is being taken according to a prescribed regimen. For example, consider a medication that is to be taken immediately before bed. If data traces reveal that the medication is taken, but also reveal that the patient then walked immediately thereafter, it may be inferred that the patient did not in fact go to bed after taking the medication, and thus did not adhere to the medication regimen.

Now with reference to FIG. 11, an example embodiment of a “smart” container 1102 adapted for collecting medication data and addressing nonadherence is shown, in cross-section view. As illustrated the container 1102 is an eyedrop bottle adapted for administering eyedrop medication. However, FIG. 11 is an example only, and other containers/configurations may be suitable.

The container 1102 includes a container wall 1103, with a cavity defined therein and adapted to contain a liquid medication 1146. The container 1102 also defines a nozzle 1106 adapted for extruding droplets of the liquid medication 1146, e.g., when pressure is applied to the container 1102. In addition, the container includes an orientation sensor 1118, a processor 1136, a data store 1138, and a communicator 1140. Although for simplicity lines of communication are not shown in FIG. 11, the orientation sensor 1118, pressure sensor 1120, data store 1138, and communicator 1140 are in communication with the processor 1136. (Power sources, power lines, etc. as may be present likewise are not shown.)

Given such arrangements, values of pressure applied to the container 1102 may be sensed by the pressure sensor 1120, and values of orientation for the container 1102 may be sensed by the orientation sensor 1118. The orientation and pressure values may be communicated to the processor 1136, which may process the orientation and pressure values as previously described herein, e.g., determining orientation and pressure traces therefrom, identifying signatures therein, determining confidences, etc. When signatures indicate that a medication event has taken place, the processor then may register the medication instance along with the time thereof (e.g., through an on-processor clock, though other arrangements may be suitable). The registered medication instance event and medication instance time then may be communicated to some recipient, such as the data store 1138, or to some recipient external to the container 1102 via the communicator 1140.

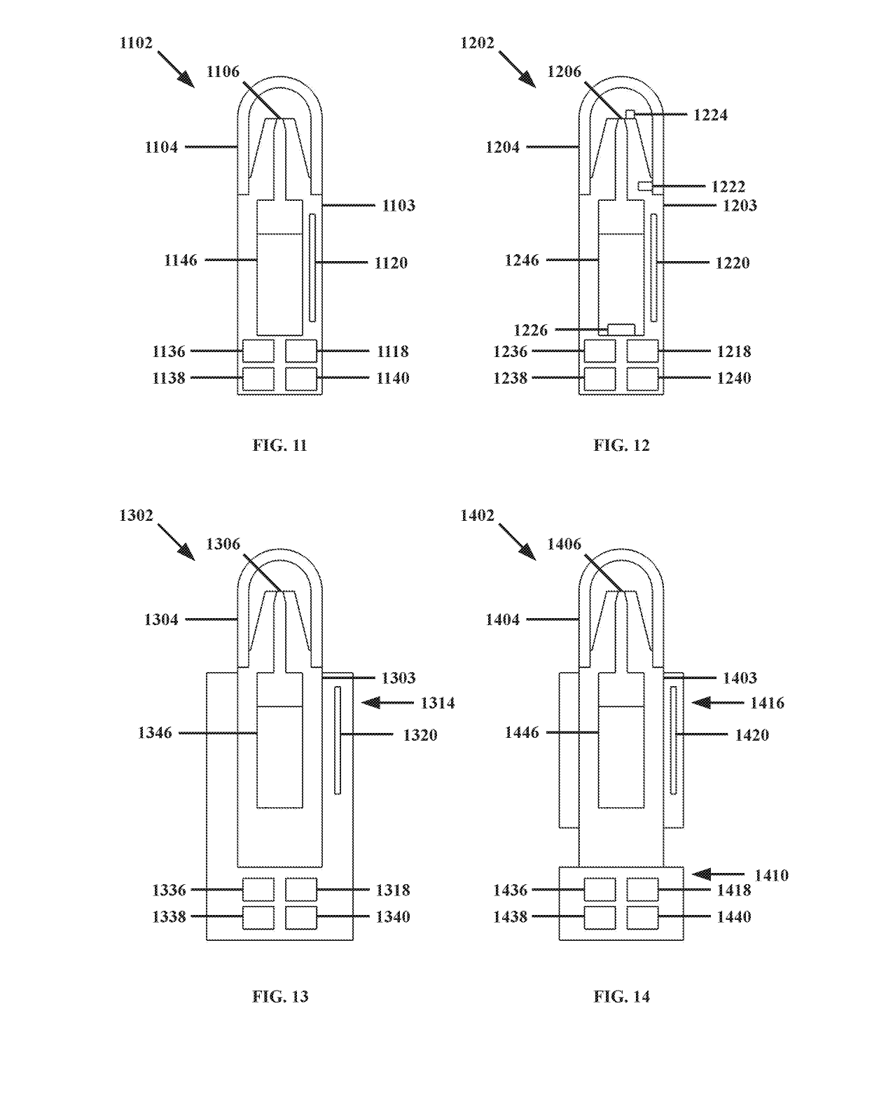

The particulars of the elements and configuration of the container 1102 may vary widely, and are not limited. Certain other example configurations are shown and described subsequently herein with regard to FIG. 12 through FIG. 15. In addition, embodiments may incorporate other containers than those shown and described, other elements, and/or other devices.

In particular, not all embodiments will have or will require all elements shown in FIG. 11. For example, an apparatus with a communicator 1140 as shown in FIG. 11 may not require, and may not necessarily include, a data store 1138. It may be suitable to communicate data (such as medication event instances) to some external recipient via the communicator 1140, without necessarily storing such data internally within a data store 1138. Similarly, other features may be present and/or absent depending on the particulars of an embodiment.

Elements as may be present in various embodiments also may vary. For example the processor 1136 is adapted to receive, process, and communicate data as described previously herein, but is not otherwise limited. A range of processors, including but not limited to digital processing chips, may be suitable.

The data store 1138 and communicator 1140 likewise may vary considerably, when present in a given embodiment. For example, the data store 1138 may be a hard drive, solid state drive, etc., while the communicator 1140 may be a simple hard-wired connection, a wired modem, a wireless system such as wi-fi or Bluetooth™, etc.

The sensors also may vary. In FIG. 11 the pressure sensor 1120 is presented as a strip or film, such as may be stressed and/or deformed when pressure is applied to the container wall 1103 so as to squeeze droplets from the container 1102. Various approaches may be suitable, such as piezoelectric layers, resistive/capacitive sandwiches, etc. Similarly, while gyroscopic sensors may be suitable for use as the orientation sensor 1118, other arrangements also may be equally suitable.

In addition, it is noted that the number and type of sensors is not limited. The arrangement of FIG. 11 shows two sensors, the orientation sensor 1118 and the pressure sensor 1120, so as to correspond with certain previously-described examples addressing orientation and pressure traces. However, sensors (and likewise data traces) are not limited only to orientation and pressure. Systems with additional sensors, and/or systems lacking either or both an orientation sensor 1118 and a pressure sensor 1120, may be suitable. Furthermore, it is not necessary to have two sensors; more and/or fewer sensors may be suitable. Typically, though not necessarily it may be useful to have two or more data traces, however in certain embodiments it may be suitable to acquire multiple data traces from a single sensor. For example, a capacitive/resistive approach for determining pressure applied to the container wall 1103 may yield a capacitance trace and a resistance trace, facilitating identification of signatures in either trace (and potentially of signatures in both traces in cooperation), even though only a single sensor is present. Arrangements in FIG. 12 through FIG. 15 also show additional sensors as may be useful in certain embodiments.

With reference now to FIG. 12, an example of a variations in sensors is shown. FIG. 12 depicts another container 1202, with a container wall 1203, a cap 1204, and nozzle 1206. The container 1202 also includes a processor 1236, data store 1238, and communicator 1240, similarly to the arrangement shown in FIG. 11.

However, while the container 1202 in FIG. 12 includes an orientation sensor 1218 and a pressure sensor 1220 also similar to FIG. 11, the container 1202 in FIG. 12 also is shown to include several other sensors. Namely, the container 1202 includes a cap sensor 1222, a droplet sensor 1224, and a quantity sensor 1226.

The cap sensor 1222 is adapted to determine whether the cap 1204 is engaged with the rest of the container 1202. For example, the cap sensor 1222 may be a force sensor that detects when a close-fitting cap 1204 is in place by determining whether physical force is being applied to the container 1202 (e.g. to some portion of the wall 1203) by the cap 1204 being physically engaged with the container 1202. As another example, the cap sensor 1222 may be a conductive sensor, determining whether a conductive path is closed by the presence of a conductive cap 1204 bridging electrical contacts (and/or a cap 1204 wherein some portion thereof is conductive, such as a conductive strip on the inside of the cap 1204, likewise bridging electrical contacts). As yet another example, the cap sensor 1222 may be an optical sensor, determining whether light is blocked (and/or dimmed or otherwise modified) by the presence of the cap 1204 on the container 1202. Other arrangements also may be suitable. However, regardless of the particulars of the cap sensor 1222, the cap sensor 1222 may then acquire a series of values forming a data trace, e.g., a “cap trace”, that may exhibit signatures consistent with the cap being present, being removed or replaced, being manipulated (e.g., by a user toying with the cap), and/or other contextual events.

As previously noted, e.g., with regard to cap removal and replacement signatures in pressure traces, the removal and/or replacement of a cap from a medication container may in at least certain instances indicate and/or suggest that a medication is about to be taken/has been taken. However, where pressure traces as shown may be adapted to provide other information (such as whether the container is being squeezed), and/or may provide cap remove information secondarily (by detecting pressure changes consistent with removing a cap), a cap trace (as from a cap sensor 1222) may indicate the status of a cap more directly. Thus, a cap sensor 1222 may be dedicated to and/or specialized for determining whether the cap is in place, is being manipulated, etc. (although a cap sensor 1222 that indicates other information also is not excluded). It is noted that the presence of a cap sensor 1222 and/or a cap trace does not necessarily exclude consideration of a pressure sensor 1220 and/or a pressure trace in determining whether the cap 1204 of a container 1202 is in place. For example, a cap trace and pressure trace could be considered in cooperation to determine whether a signature of cap removal/replacement is exhibited (e.g., suitable pressure is applied to the bottle as shown in the pressure trace, corresponding with a form in the cap trace indicating that the cap is no longer in place, etc.).

The droplet sensor 1224 is adapted to determine whether a droplet is present and/or is exiting the nozzle 1206. For example, the droplet sensor 1224 may be a conductive sensor; if a conductive liquid passes between electrical contacts, the electrical conductivity between those contacts may change, thus potentially providing an indication that a droplet has been expelled from the container 1202. Other electrical variations, such as changes in capacitance, inductance, etc. may also be suitable (e.g., particularly if the medication itself is not distinctively conductive or resistive electrically, and so does not provide a clear indication of presence through a measurement of resistance and/or conductivity). As another example, the droplet sensor 1224 may be an optical sensor, determining whether light in the vicinity of the nozzle 1206 is obstructed, which may serve as an indication that a droplet has passed through the nozzle 1206. As yet another example, the droplet sensor may be acoustic, detecting the sound of a droplet being extruded from the nozzle 1206, etc. Other arrangements also may be suitable.

As also has been previously noted, the exit of a droplet from a container may be suggestive and/or indicative of a medication being dispensed. As noted with regard to the cap sensor 1222, the droplet sensor 1224 in FIG. 12 may provide more direct information regarding droplets being expelled than (for example) a pressure sensor, but such is not required, nor is a droplet sensor 1224 limited only to detecting a droplet. For example, an optical sensor used as a droplet sensor 1224 may also serve as a cap sensor 1222; an optical trace from a sensor disposed near the nozzle 1206 may show signatures of both cap presence (e.g., by variations in light level due to the cap 1204 being present/absent) and droplets exiting the nozzle 1206 (e.g., by variations in light levels characteristic with a droplet interfering with an optical path).

The quantity sensor 1226 is adapted to determine a quantity of medication present in the container 1202, and/or a change of quantity of medication therein. For example, a pressure sensor may determine the amount of medication present (and/or changes in quantity) based on the weight of that medication. Alternately, an electrical sensor may determine the amount of medication present (and/or changes in quantity) based on electrical properties of medication within the container 1202. Other arrangements also may be suitable.

As may be understood, the quantity of medication within a container 1202 over time may be of use in determining whether a medication is used, when that medication is used, the dosage of that medication that is used, etc. Thus, a medication quantity trace may exhibit signatures that may be of interest, in addition to and/or in place of pressure and orientation traces as previously described. In addition, a medication quantity trace also may reveal additional information, e.g., a medication quantity sensor 1226 may indicate that medication in a container 1202 is running low, and provide some indication (e.g., to the user, to a pharmacy, etc.) that a refill may be needed.

Thus, the arrangement shown in FIG. 12 shows five sensors: the orientation sensor 1218, the pressure sensor 1220, the cap sensor 1222, the droplet sensor 1224, and the quantity sensor 1226. At one trace per sensor (which as noted previously is not limiting; multiple sensors may cooperate to produce a trace, and a single sensor may product two or more traces) the five sensors in the container 1202 may provide five data traces. Signatures may be identified in any of the sensors 1218 through 1226, and in any combination of two or more sensors 1218 through 1226 in cooperation. Speaking more broadly, in a given embodiment certain sensors may be general (e.g., the pressure sensor 1220 as may provide signatures consistent with numerous events, including but not limited to medication events and contextual events), while others may be specific to a particular property (e.g., the droplet sensor 1224 which in certain embodiments may provide dedicated information regarding only whether a droplet has exited the nozzle 1206, though as noted in some embodiments the droplet sensor 1224 may indeed provide other information). Cooperation among traces in identifying signatures also may take place in many combinations, e.g., orientation and pressure to determine if a droplet has been expelled (as shown in previous example traces), pressure and droplet likewise to determine if a droplet has been expelled, pressure and cap to determine whether the cap 1204 has been removed, etc. Other arrangements also may be suitable, and embodiments are not limited with regard to what sensors may be present, what properties those sensors may detect, how many sensors may be present, which sensors cooperate for which signatures, etc.

As may be seen, in FIG. 11 and FIG. 12 certain elements therein—for example the sensors, processor, data store, and communicator—are shown as being integrated into the wall of the containers themselves. Thus, a container 1202 such as shown in FIG. 12 may be constructed as an “all-in-one” form, with sensors 1218 through 1226, processor 1236, etc. “built-in” to the container 1202 itself. While such integration is not prohibited and may be present in some embodiments, neither is such an arrangement necessary or limiting.

For example, turning to FIG. 13 another arrangement is shown for a container 1302. The container 1302 includes a cap 1304, wall 1303, and nozzle 1346, and holds a quantity of medication 1346. However, unlike the arrangements in FIG. 11 and FIG. 12 the container 1302 in FIG. 13 does not have sensors or other elements incorporated into the container 1302 itself. Rather, a jacket 1314 is disposed around the container 1302 (or alternately, the container 1302 may be understood as being disposed within the jacket 1314).

In the arrangement of FIG. 13, the jacket 1314 incorporates the orientation sensor 1318, the pressure sensor 1320, the processor 1336, the data store 1338, and the communicator 1340. Thus, the jacket 1314 may be produced separately and then engaged with the container 1302. For certain embodiments such engagement may be permanent, such as by gluing or welding the jacket 1314 into place so that the container 1302 cannot be removed therefrom. In other embodiments engagement may be temporary and/or removable, such that a container 1302 may be inserted within the jacket 1314, and that container 1302 later removed; depending on the particulars, the same or another container 1302 then could be inserted into the jacket 1314 (and/or a different jacket 1314 could be applied to the container 1302). As the elements needed for acquiring traces (e.g., sensors 1318 and 1320) are disposed within the jacket 1314 in such embodiments, an arrangement as shown in FIG. 13 thus may enable jackets 1314 to be manufactured separately from medication containers 1302, with the jackets 1314 serving to retrofit certain capabilities as described herein onto “dumb” containers. The arrangement in FIG. 13 may also enable reuse of a single jacket 1314 with many containers 1302 over time. Such arrangement may avoid costs associated with producing sensors, processors, etc. for each individual container, may facilitate ongoing data collection and processing even as many containers are emptied (for example if a new container is obtained with each refill of a prescription, etc.), and so forth.

Now with reference to FIG. 14, embodiments wherein certain “smart” elements are separate from the container proper are not limited only to the use of a jacket, and also it is not required that all such “smart” elements (e.g. the sensors, processor, etc.) must be in or on the same body. For example, FIG. 14 shows a container 1402 with a cap 1404, a wall 1403, a nozzle 1446, and a medication 1446. As in FIG. 13, certain elements shown in FIG. 14 are distinct from the container 1402 proper.

However, where FIG. 13 depicts a single jacket 1314, FIG. 14 shows a shoe 1410 and an annex 1416, in the form of a membrane 1416 wrapped around some portion of the container 1402. (While for explanatory purposes the terms annex and membrane may be used interchangeably for the example of FIG. 14, in practice annexes are not limited only to membranes; bodies other than membranes engaged with containers, e.g., with sensors therein/thereon, also may serve as annexes.) The membrane 1416 includes a pressure sensor 1420, while the shoe 1410 includes an orientation sensor 1418, a processor 1436, a data store 1438, and a communicator 1440. The membrane 1416 is adapted to be engaged (fixedly or removably) with the wall 1403 of the container 1402, for example in the form of an adhesive label wrapped around the container 1402. In such instance the pressure sensor 1420 may be disposed inside the membrane 1416, on an inner surface thereof, on an outer surface thereof, etc. The shoe 1410 in turn is adapted to be engaged (again fixedly or removably) with the bottom of the container 1402. The orientation sensor 1418, processor 1436, data store 1438, and communicator 1440 may be on a surface of or integrated into the shoe 1410, may be disposed within an interior compartment of the shoe 1410, etc.

As noted with regard to FIG. 13, the arrangement in FIG. 14 may facilitate certain elements to be provided separately from the container 1402, for example the pressure sensor 1420 may be provided separately e.g., being laminated into a membrane 1416 in the form of an adhesive label that is later applied to the container 1402. Likewise, the shoe 1410 may be provided separately (along with elements therein/thereon), and then engaged with the container 1402 as well. As previously noted, such arrangements may enable elements to be reused, to be made non-disposable even if a container 1402 is disposable, to be retrofitted to a container not originally adapted to be “smart”, etc. Also, certain “smart” elements may be made separately from others, e.g., the pressure sensor 1420 may be in the membrane 1416 while the processor 1436 is in the shoe 1410. (It is noted that in such instances, it may be necessary or useful to engage the membrane 1416 with the shoe 1410, for example through a connecting wire. Such wiring or other connections are not shown in FIG. 14 for purposes of simplicity.)

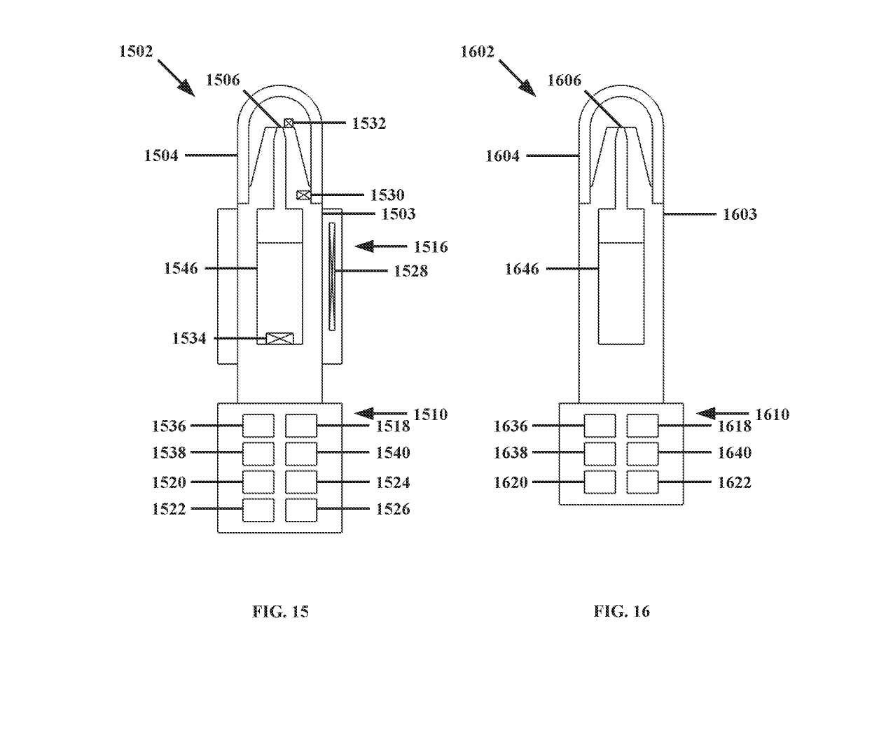

In addition, with reference now to FIG. 15 it may be suitable to subdivide certain elements. FIG. 15 shows a container 1502 with a cap 1504, a wall 1503, a nozzle 1546, and a medication 1546. However, in comparison with FIG. 13, which shows sensors 1320, 1322, 1324, and 1326 distributed across the container 1302, in FIG. 15 different elements are disposed in similar positions. Namely, FIG. 15 shows a pressure probe 1528 disposed in a membrane 1516, a cap probe 1530 at a point of contact between the container 1502 and cap 1504, a droplet probe 1532 proximate the nozzle 1506, and a quantity probe 1534 inside the container 1502 within the medication 1546. FIG. 15 still depicts a pressure sensor 1520, cap sensor 1522, droplet sensor 1524, and quantity sensor 1526; however, all of these sensors proper 1528 through 1526 are disposed within a shoe 1510 (along with orientation sensor 1518, processor 1536, data store 1538, and communicator 1540).

In essence, sensors 1520 through 1526 are each split into parts, with some parts disposed on the container 1502 and other parts disposed within the shoe 1510. For example, in certain embodiments it may be useful for the container 1502 to be disposable once the medication is exhausted (or expired, no longer needed, etc.), while the shoe 1510 is reusable with multiple containers of the same medication, multiple medications, etc. In such circumstance, it may be useful to dispose parts of the various sensors that are expensive, difficult to recycle, etc. within the reusable shoe 1510 rather than the disposable container 1502. Considering pressure sensing as an example, the pressure probe 1528 in the membrane 1516 (the membrane 1516 being disposable with the container 1502 for this example) may include only such parts as may be required to be on/near the container 1502. As a more concrete example, for an electronic resistance/capacitance pressure sensor, the resistance/capacitance sheet (e.g., conductive layers with a dielectric therebetween) may serve as the probe 1528, and may be disposed within the membrane 1516; by contrast, whatever electronics that measure resistance and capacitance within the pressure probe 1528, that pre-process the pressure data, that record the pressure data, etc., may be within the pressure sensor 1520 in the shoe 1510. Thus, when the container 1502 is disposed of, only the pressure probe 1528 is lost, which may be relatively simple, inexpensive, non-hazardous, etc. as compared with the pressure sensor 1520 overall. Likewise, the cap probe 1530 may be merely electrical contacts, the droplet probe 1532 merely an optical path, and the quantity probe 1534 merely a piezo crystal; the cap sensor 1522, droplet sensor 1524, and quantity sensor 1526 would be retained in the shoe 1510 when disposing of the container 1502. In such case, even if the sensors were numerous and sophisticated, it may be suitable for the container 1502 to be disposable, since the parts that are expensive, etc. may be disposed within the shoe 1510 which in this example would be retained and reused, along with the orientation sensor 1518, processor 1536, data store 1538, and communicator 1540.

It is noted that even when one or more sensors or other elements may be subdivided, it is not required that all sensors or elements must be subdivided. For example, the orientation sensor 1518 has no probe in FIG. 15. For example, a gyroscopic orientation sensor 1518 may not require contact or other phenomena that might benefit from a separate probe; if the orientation sensor 1518 is in the shoe 1510, and the shoe 1510 moves with the container 1502 (being engaged therewith), the orientation of the container 1502 may be inferred from the orientation of the shoe 1510; thus a probe may not provide advantage.

For embodiments that include a shoe 1510, the elements that may be disposed therein are not limited. All, some, or none of each sensor as may be present may be within the shoe 1510. All, some, or none of the processor, data store, and communicator (when present) may be within the shoe 1510. Distinctions may be made such as “all active components are disposed within the shoe 1510”, “all electronic components”, “all components over a given cost”, “all components not readily recycled”, etc. In addition, all, some, or none of any additional elements (e.g., not illustrated in FIG. 15) may be within the shoe 1510, such as a power source, electrical leads, optical pathways, etc.

Now with reference to FIG. 16, while certain previous examples have shown sensors that are on the container proper as opposed to on a foot, in a jacket, etc., this is not necessarily required of all embodiments. For example, as shown in FIG. 16 a container 1602 includes a cap 1604, a wall 1603, a nozzle 1646, and a medication 1646 (and/or other contents). A shoe 1610 is also shown engaged with the container 1602; the shoe 1610 includes an orientation sensor 1618, processor 1636, data store 1638, and communicator 1640. In addition, the shoe includes a pressure sensor 1620 and a cap sensor 1622. Thus, all sensors shown in FIG. 16 are in the shoe 1610; the container 1602 itself is not required to incorporate sensors. Such an arrangement may be useful for example in retrofitting existing “dumb” containers. That is, a shoe 1610 may be added to an existing container 1602, enabling authenticated collection of data and/or use thereof in addressing nonadherence, as described herein, without otherwise requiring the container 1602 to be modified, and/or without requiring any other special features on the container 1602.

Such an arrangement as shown in FIG. 16 may utilize sensors that do not require direct physical engagement with relevant elements of the container 1602. For example, as noted previously an orientation sensor 1618 such as a gyroscopic sensor may provide orientation data for the container 1602 even if not directly on the container 1602; if the shoe 1610 is engaged with the container 1602 and the orientation of the shoe 1610 may be determined by the orientation sensor 1618, the orientation of the container 1602 then may be inferred. Likewise, a sensor such as an ultrasonic sensor may be suitable for use as a pressure sensor 1620 as shown in FIG. 16; changes in the shape of the container 1602 such as may manifest when the container 1602 is squeezed may change the acoustical properties of the interior of the container 1602, and thus potentially may be recognized as signatures of pressure being applied to the container 1602 (e.g. to squeeze out a droplet). It is noted that in such chase, pressure is not necessarily being measured directly, rather the existence and degree of pressure may be inferred from the changes observed by an ultrasonic sensor serving as a pressure sensor 1620.

It is emphasized that such indirect determinations of data such as pressure, orientation, etc. are not prohibited generally, and may be suitable for other sensors and/or properties as well. Thus, an embodiment may acquire data regarding “pressure”, form traces for pressure, identify pressure-related signatures, etc., even without necessarily measuring pressure per se. So long as a phenomenon produces a useful signature, that phenomenon may be considered for purposes as described herein.

Still with reference to FIG. 16, and continuing in an example of sensors not necessarily in contact with the container 1602 yet acquiring data regarding the container 1602, a cap sensor 1622 may take the form of a light sensor. For example, if the container 1602 were transparent/translucent while the cap 1604 were opaque, or if the nozzle 1606 may be expected to pass some amount of light therethrough, then removing the cap 1602 may change the amount of light reaching a cap sensor 1622 that is disposed within the shoe 1610, e.g., a light sensor looking upward into/through the container 1602. Thus the status of the cap 1604 may be determined by a cap sensor 1622 that is not in contact with the cap 1604, or necessarily even in direct contact with any portion of the container 1602.

Other arrangements also may be suitable.

With regard collectively to FIG. 11 through FIG. 16, for explanatory purposes a distinction is made in places between “container” and “jacket”, “shoe”, “annex”, and/or “membrane”, and between “sensor” and “sensor probe”. However, in practice it may not be inaccurate to refer to an assembly of container and jacket (e.g., as shown in FIG. 13) collectively as “the container”. For example, from the point of view of a patient, when one picks up and squeezes medication from the container 1302, the jacket 1314 also moves and is manipulated therewith. Thus, references to “a container” herein may refer equally to an integral arrangement as shown in FIG. 11, a container with a jacket as shown in FIG. 13, a container with membrane and/or shoe as in FIG. 15, other arrangements etc., and should not be interpreted as limiting. Likewise, from the point of view of the patient the distinction between “sensor” and “sensor probe” may be inconsequential in practice, and thus references to “a sensor” may refer equally to an integral sensor, a sensor distributed in components (e.g., a sensor and a probe therefor), etc., and also should not be interpreted as limiting.

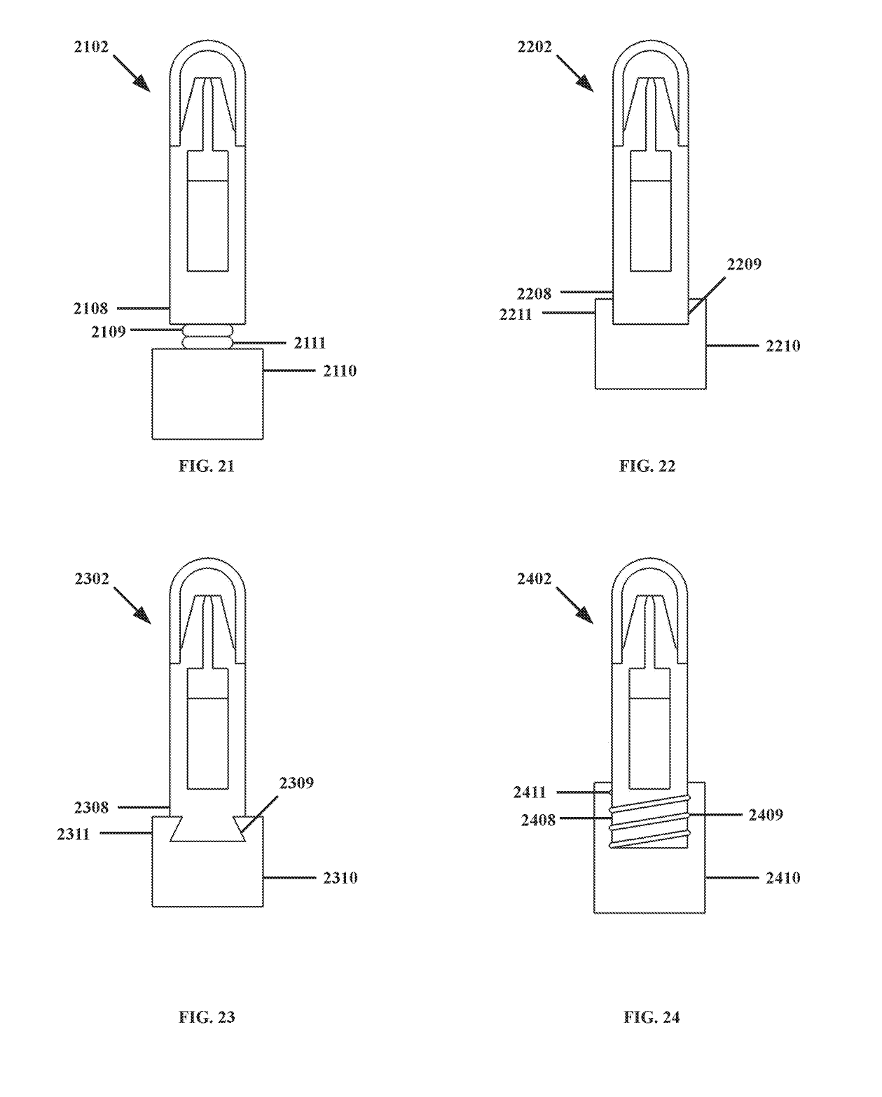

In addition, while a distinction is made in certain instances herein between various entities engaged with a container, the terminology used for such entities should not be understood as limiting. For example, while FIG. 13 shows a jacket 1314 encompassing a container 1302, and FIG. 14 shows a shoe 1410 engaged with the bottom of the container 1402 therein. The difference in practice, if any, is at least largely descriptive; a container fits “in” a jacket but “on” a shoe. A jacket need not fully enclose a container, or continuously enclose even part of a container; for example a jacket may have apertures therein, may extend only halfway up a container, may have no bottom (e.g., being a simple sleeve with no ends), etc. Conversely, a shoe may cover and/or enclose some portion of a container, e.g., accepting the container into a socket defined in the shoe (as is shown in FIG. 22, below). Other arrangements also are suitable; the distinction between “jacket” and “shoe” is thus illustrative, but is not necessarily limiting.

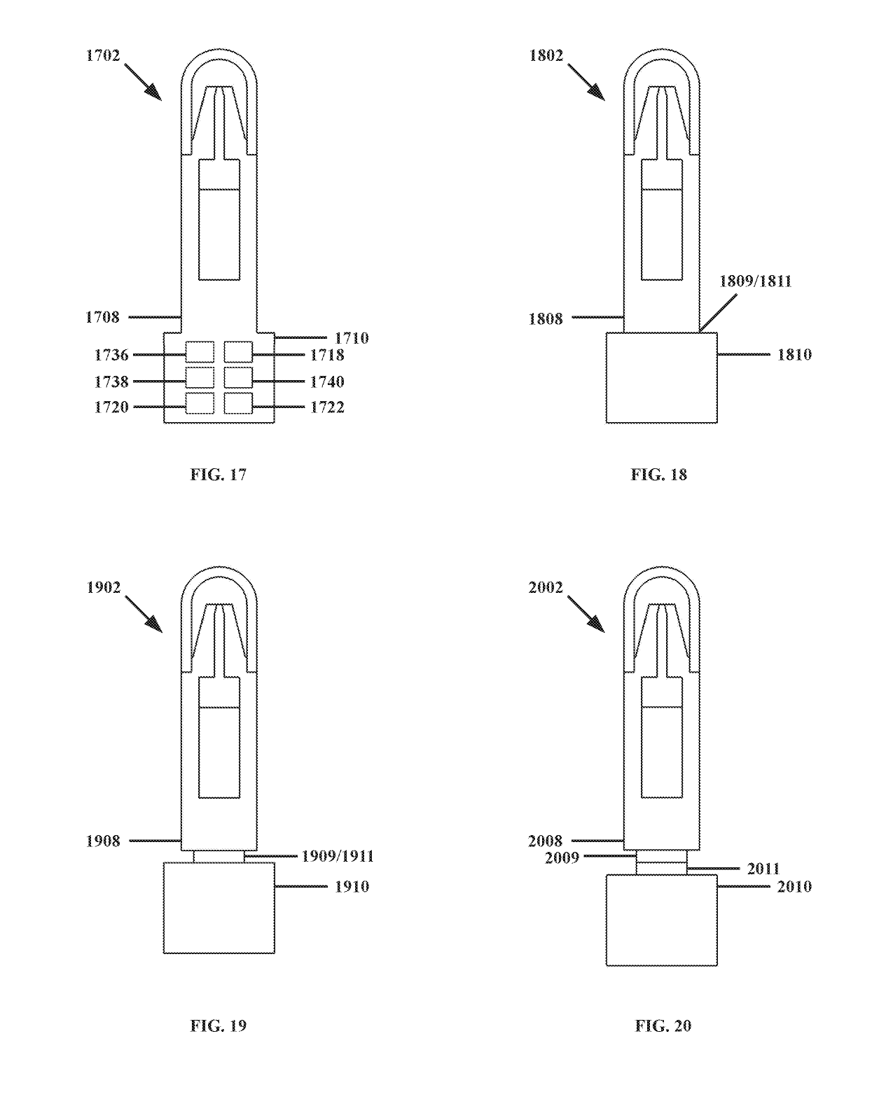

With reference now to FIG. 17 through FIG. 33, the arrangements for incorporating sensors and/or other elements with containers, and in particular engaging shoes with such sensors and/or other elements with containers, may vary considerably and are not limited. Broadly speaking, a “shoe” may be understood as some assembly of elements such as sensors, processor coupling, etc. as may be engaged (in a variety of ways) with a container. A “foot” may be understood as that portion of the container with which the shoe is engaged. In certain instances, the foot itself may be distinct from the rest of the container, and thus the foot may be engaged with the container as well as the shoe being engaged with the foot.

For example, FIG. 17 shows a container 1702. The container 1702 is integral; the portion shown to contain the processor 1736, data store 1738, communicator 1740, and orientation, pressure, and cap sensors 1718, 1720, and 1722 is continuous with the remainder of the container 1702. For example, the container 1702, sensors 1718, 1720, 1722, etc. may be formed together, e.g., as a continuous injection-molded plastic form (with sensors embedded therein). As such, while a foot 1708 and a shoe 1710 are shown for purposes of reference and comparison with other illustrations, describing such elements as a distinct foot and shoe may be academic, as the container 1702 is shown to be a single integrated structure. Thus, while arguably the foot 1708 and shoe 1710 may be said to be coupled together, no distinct couplings are identified. Such an integrated form may be suitable for certain embodiments.

As an aside, for simplicity and clarity in showing variations in arrangements of container, foot, shoe, etc., certain elements of/relating to such structures are not independently illustrated in FIG. 18 through FIG. 34. For example, while FIG. 17 shows a processor 1736, data store 1738, communicator 1740, and orientation, pressure, and cap sensors 1718, 1720, and 1722, corresponding features are not shown in all of FIG. 18 through FIG. 34; such features may be present in embodiments, however (as may be additional features such as wires, power sources, etc., as also may not be particularly illustrated).

With reference to FIG. 18, a container 1802 is shown with a foot 1808 and a shoe 1810. The shoe 1810 is distinct from the remainder of the container 1802, e.g., the container 1802 and shoe 1810 may have been fabricated separately, but the shoe 1810 has been engaged with the container 1802 so as to be a near-continuous structure. The foot coupling 1809 and shoe coupling 1811 may be a weld joint, fusion joint, etc.; while not as fully integral as the arrangement in FIG. 18, the foot 1808 and shoe 1810 in FIG. 18 are so joined that the coupling therebetween may essentially amount to unifying the shoe 1810 and the foot 1808 into one structure. Such coupling 1809/1811 typically (though not necessarily) may be permanent in nature, e.g., solvent welding or heat fusing two plastic parts together, welding or soldering metal parts, etc.

By contrast, FIG. 19 shows a container 1902 wherein the foot 1908 and the shoe 1910 are engaged, but wherein a foot/shoe coupling 1909/1911 may be at least visible. For example, the foot/shoe coupling 1909/1911 may be an adhesive bond, with the shoe 1910 having been made separately from the remainder of the container 1902 and later glued thereto. Such an arrangement may be, but is not required to be, permanent. For example, while certain adhesives may form a permanent coupling 1909/1911, others such as silicone pressure-sensitive adhesive films may be separable, and or may enable parts to be removed and then re-attached and/or attached to other parts.

FIG. 20 shows another arrangement, with a container 2002 having a shoe 2010 coupled to the foot 2008 thereof. The engagement of the shoe 2010 to the foot 2008 exhibits distinct couplings 2009 and 2011 for the foot 2008 and the shoe 2010 respectively; for example, if collectively the coupling 2009/2011 is hook-and-loop material, the foot coupling 2009 may be a patch of hook material while the shoe coupling 2011 may be a patch of loop material. Such an arrangement as shown in FIG. 20 may be (but is not required to be) removably engaged, e.g., so as to enable conveniently removing the shoe 2010 to be engaged with the foot 2008 of various containers 2002 as needed.

Similarly, in FIG. 21 another arrangement is shown for a container 2102 that has a shoe 2110 coupled to the foot 2108 thereof. The foot coupling 2109 and shoe coupling 2111 are illustrated as snap fittings, such that the foot 2110 would snap on to the container 2102. Such an arrangement again may be removably engagable.

Turning to FIG. 22, the container 2202 therein is shown fitted into the shoe 2210. The shoe 2210 defines an aperture therein, with a lip serving as a shoe coupling 2211. The foot 2208 fits into to the aperture, so that the lower end of the foot 2208 serves as the foot coupling 2209. The arrangement shown in FIG. 22 may be seen as an example of a friction fit: the aperture in the shoe 2210 is sized (or may be deformable, constructed with spring-loading, etc.) such that the foot 2208 fits tightly inside the aperture, friction (and/or spring force, etc.) between the foot 2208 and the shoe 2210 holding the two together. Such an arrangement may be, but is not required to be, removably engagable.

In FIG. 23, a container 2302 is shown again fitted into the shoe 2310. The shoe 2310 again defines an aperture therein with a lip serving as the shoe coupling 2311, and the foot 2308 fits into the aperture so that the lower end of the foot 2308 serves as the foot coupling 2309. However, where in FIG. 22 the coupling 2209/2211 engages with a friction fit, in FIG. 23 the coupling 2309/2311 utilizes shape-locking: as may be seen, the lip serving as the shoe coupling 2311 is angled inward so that the top of the aperture is narrower than the bottom, while the foot coupling 2309 is in turn wider at the bottom than at the top. Consequently, once the foot coupling 2309 is engaged with the shoe coupling 2311, the respective shapes of the foot and shoe couplings 2309 and 2311 hold the shoe 2310 engaged with the container 2302. Such an arrangement may be, but is not required to be, removably engagable; if the materials of the foot 2308 and shoe 2310 are sufficiently flexible the two may separate if pulled, but if relatively rigid the join may be essentially permanent.

FIG. 24 shows yet another arrangement, with a container 2402 fitted within a shoe 2402. (It is noted again, as indicated previously, that a distinction between “shoe” and “jacket” may be at least largely a matter of chosen terminology. As may be seen in FIG. 24, the shoe 2410 extends some distance up the sides of the container 2402, and thus may be said to enclose at least a portion of the container 2402. It may be equally suitable to refer to the shoe 2410 as a jacket, or by some other term.) As may be seen, the shoe 2410 defines an aperture with grooves to accommodate threads serving as the shoe coupling 2411. The foot 2408 in turn includes raised threads serving as the foot coupling 2409. The shoe 2410 is engaged with the foot 2408 by threading the shoe 2410 onto the foot 2408. Such an arrangement may be, but is not required to be, removably engagable.

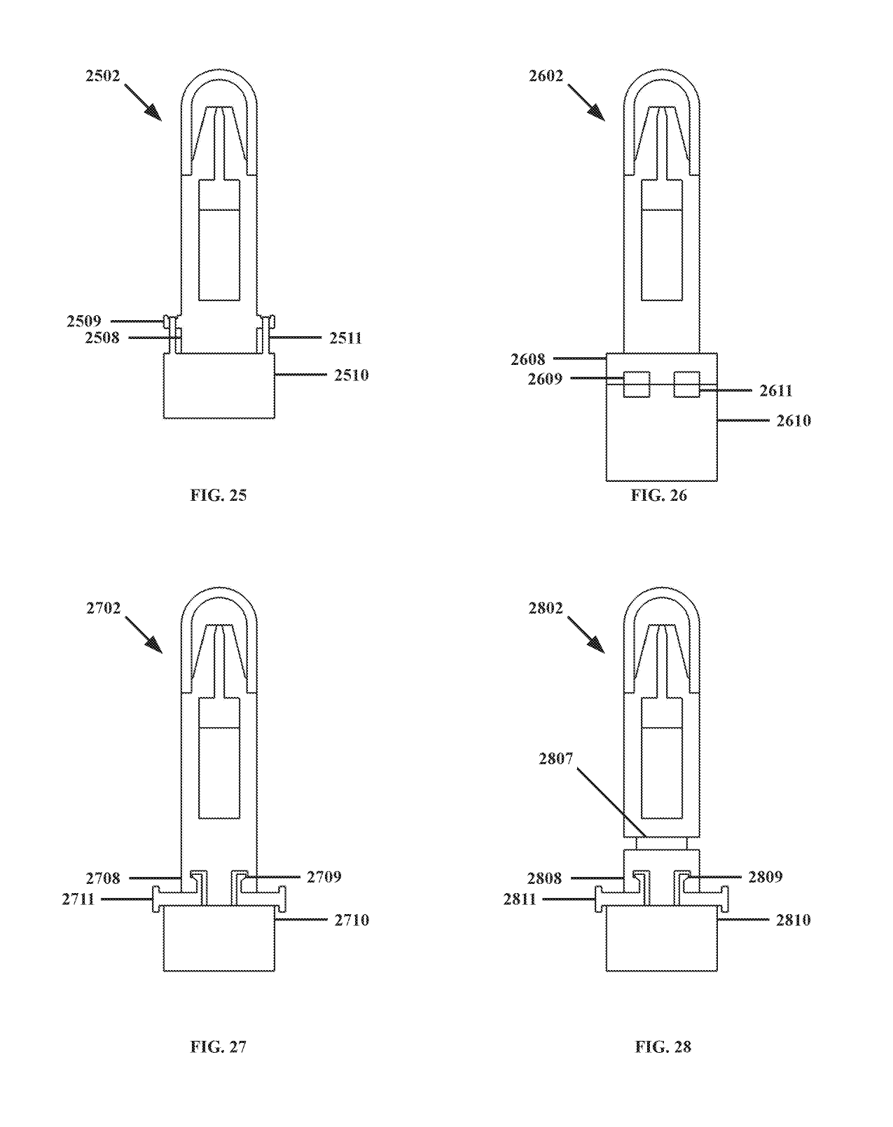

In FIG. 25 a container 2502 is again shown with a shoe 2510 engaged with the foot 2508 thereof. Mechanical fasteners in the form of loops (e.g., elastic loops) serve at shoe couplings 2511, engaging with pins serving as foot couplings 2509. That is, the loops hook over the pins, engaging the shoe 2510 to the foot 2508. Such an arrangement may be, but is not required to be, removably engagable. In addition, it is noted that fasteners may vary widely beyond the loop-and-pin arrangement in FIG. 25. For example, sliding pins, screws, nuts and bolts, hooks, etc. may be suitable in various embodiments.

FIG. 26 shows yet another arrangement, with a container 2602 having a shoe 2610 engaged with the foot 2608 thereof. The arrangement of FIG. 26 is magnetically engaged. The shoe couplings 2611 and foot couplings 2609 may be magnets, oriented so as to attract one another. Alternately, one of each pair of shoe couplings and foot couplings 2611 and 2609 may be a magnet, while the other of each such pair may be a magnetic material such as steel. Such an arrangement may be, but is not required to be, removably engagable. It is also noted with regard to FIG. 26 that the foot 2608 is shown as being distinct from the remainder of the container 2602. As previously noted this may be suitable for certain embodiments. For example, a foot 2608 may be retrofitted onto an existing “dumb” container 2602, facilitating the engagement of a shoe 2610 to support collection of data and use thereof with regard to the container.

Turning to FIG. 27, the first of several arrangements with similar mechanical latches is shown. The container 2702 is shown with a shoe 2710 engaged with the foot 2708 of the container 2702. Sliding mechanical latches serve as shoe couplings 2711; the foot 2708 in turn defines apertures therein that accept the latches therein, the apertures serving as foot couplings 2709. With the latches pushed inward towards the middle of the foot 2708, the shoe 2710 may slide vertically onto and/or off of the foot 2708. With the latches pushed outward (as shown) the latches engage with the apertures, so that the shoe 2710 may not freely slide onto and/or off of the foot 2708. In certain embodiments it may be useful to bias the latches in some manner, e.g., using springs (not shown) to keep the latches in the securing position shown in FIG. 27. Such an arrangement may be, but is not required to be, removably engagable.

FIG. 28 is at least somewhat similar to FIG. 27, with a container 2802, shoe 2810, foot 2808, and shoe and foot couplings 2811 and 2809. However, where FIG. 27 shows a foot 2708 that is integral with the remainder of the container 2702, in FIG. 28 the foot 2808 is distinct from the remainder of the container 2808. A foot connector 2807 is shown between the container 2802 and the foot 2810; the foot connector 2807 may for example be an adhesive, removable or otherwise. As noted previously, the foot for a given embodiment (not merely that in FIG. 28 or other illustrations depicting such a feature) may be distinct from the remainder of a container, and/or may be removable.

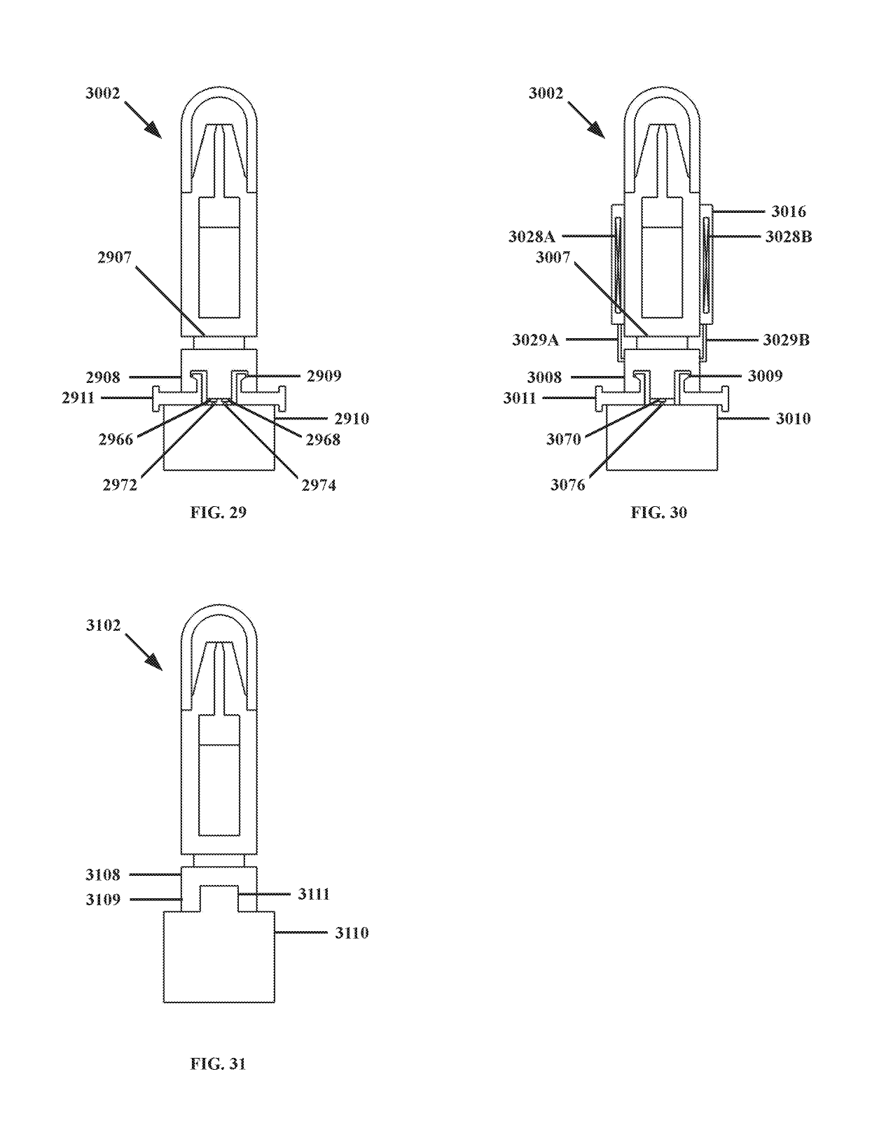

FIG. 29 in turn is at least somewhat similar to FIG. 28, with a container 2902, shoe 2910, foot 2908, shoe and foot couplings 2911 and 2909, and foot connector 2907. However, in addition FIG. 29 shows a foot data port 2966 and a foot power port 2968 on the foot 2908, and a corresponding shoe data port 2972 and shoe power port 2974 on the shoe 2910. As shown and described previously, elements such as sensors, etc., may be present on a container outside of the foot thereof. Consequently, it may be necessary or at least useful to communicate electrical power to such elements from the foot, e.g., via the shoe power port 2974 delivering power to the foot power port 2968 (or vice versa). Likewise it may be necessary or at least useful to communicate data from such elements to the foot, e.g., via the foot data port 2966 delivering data to the shoe data port 2972 (or vice versa). The ports 2966, 2968, 2972, and 2974 are illustrated as simple point-and-pad electrical conductors, but this is an example only, and other arrangements may be suitable. A wide range of electrical connections, wireless connections, optical connections, etc. may be suitable for various ports. In addition, embodiments are not limited to only one port of each type (power and data) on the shoe 2910 and/or foot 2908, nor only to ports for power and/or data.

FIG. 30 is again at least somewhat similar to FIG. 29, with a container 3002, shoe 3010, foot 3008, shoe and foot couplings 3011 and 3009, and foot connector 3007. However, where FIG. 29 shows separate data and power ports, FIG. 30 illustrates a combined foot data/power port 3070 and a combined shoe data/power port 3076. Various combinations of dedicated power and data ports, and/or combined data/power ports, and/or other ports, may be suitable in various embodiments.

In addition, FIG. 30 shows an annex 3016 in the form of a membrane covering a portion of the container 3002. The annex 3016 has first and second pressure probes 3028A and 3028B therein (similar to that previously shown in FIG. 15). Also shown are pressure probe connectors 3029A and 3029B. For example, in the arrangement shown in FIG. 30, the membrane 3016 may be a label applied to the container 3002, with pressure probes 3028A and 3028B disposed therein, with connectors 3029A and 3029B connecting the probes 3028A and 3028B with the foot 3008, and the data/power ports 3070 and 3076 likewise connecting the foot 3008 with the shoe 3010. Thus a pressure sensor and power source (not shown in FIG. 30) in the shoe 3010 could receive data and provide power respectively. As has been noted previously, such an arrangement again facilitates the retrofitting of a “dumb” container with data collection and processing capabilities as described herein. For example, a “smart” label, a foot, and a shoe may be applied to a “dumb” container.

Moving on to FIG. 31, therein yet another arrangement is shown, with a container 3102 having a shoe 3110 engaged with the foot 3108 thereof. Though visually somewhat similar to the arrangement in FIG. 22, the coupling arrangement in FIG. 31 may be seen as being reversed: the foot 3108 defines an aperture with the rim thereof serving as a foot coupling 3109, while the shoe 3110 has a projection extending upward that serves as a shoe coupling 3111. The arrangement as shown in FIG. 31 thus may represent another variation on friction fitting. Again, such an arrangement may be, but is not required to be, removably engagable.

It is emphasized that numerous variations beyond what is specifically described and shown herein may be suitable.

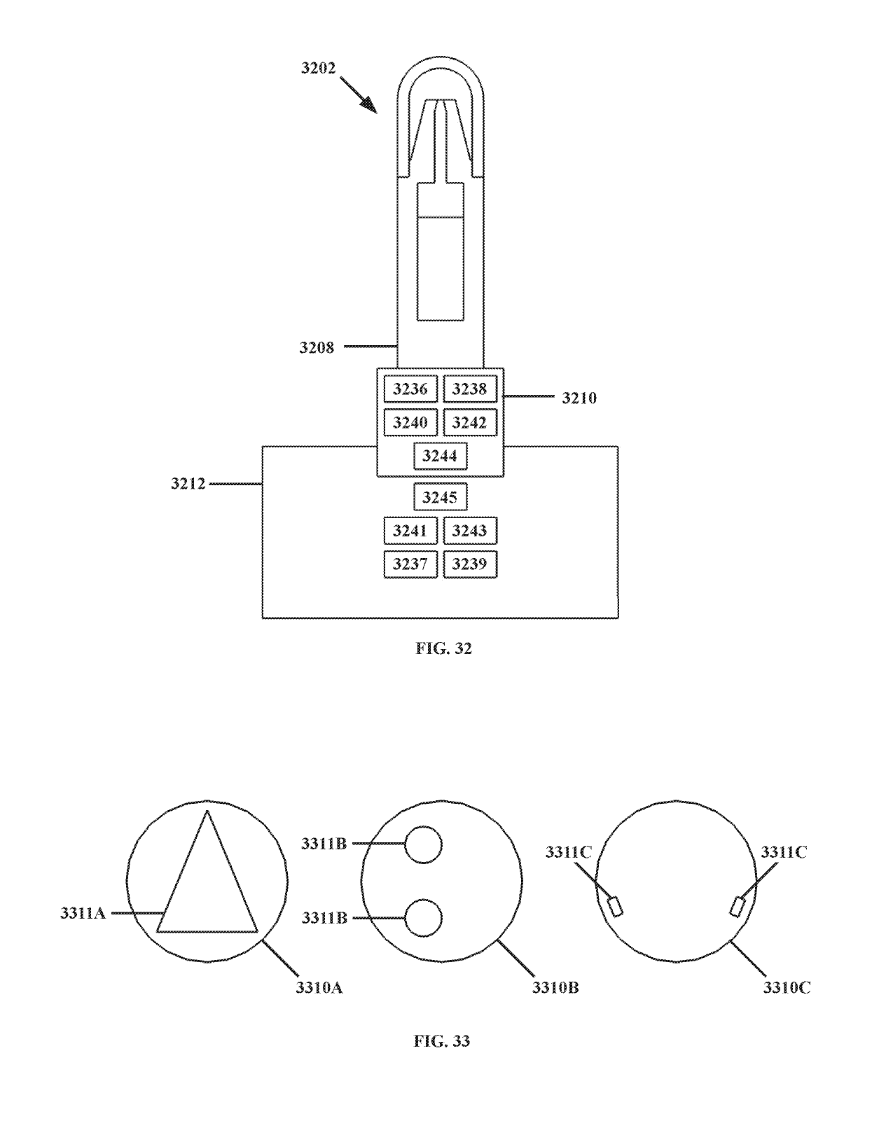

Now with regard to FIG. 32, while certain examples herein have focused on containers, shoes therefor, associated sensors, etc., it is noted that numerous other elements may be incorporated into and/or utilized with such containers, shoes, etc. FIG. 32 shows a container 3202, with a foot 3208 engaged with a shoe 3210. In addition, the container 3202 with the shoe 3210 engaged is disposed on a base 3212. The base may serve a variety of functions, with a range of elements therein/thereon.

For example, the base 3212 may include a power supply 3243 and/or charger 3245, as shown. Even if the shoe 3210 includes a power supply 3242, it may be useful to keep the total mass of the shoe 3120 low, and thus perhaps to make the power supply 3242 small to trade off limited operating life in exchange for low mass; a charger 3244 in the shoe 3210 could recharge the shoe power supply 3242 from the base power supply 3243 via the base charger 3245. The container 3202 thus could be carried for some period (e.g., during the day), used as needed, and then placed on the base 3212 for recharging (e.g., at night).

As another example, the base 3212 may include a communicator 3241. Again, even if the shoe 3210 includes a communicator 3240, it may be useful for that shoe communicator 3240 to have limited range, power, etc. A communicator 3240 inside the shoe 3210 may handle short-range and/or low-bandwidth communication (with the base 3121 or with other recipients), while a more powerful communicator 3241 inside the base 3212 may handle long-range and/or high-bandwidth communication. Alternately, the shoe communicator 3240 may have wireless and direct modes with wireless having lower bandwidth, or a direct mode only; in such case the shoe 3210 may collect data and store the data, sending little or no data to the base 3212 while the shoe 3210 is separate but transferring more data to the base 3212 when the shoe 3210 is placed thereon. Thus, either or both the shoe communicator 3240 and the base communicator 3241 may be adapted to communicate only with one another, or more widely with a range of recipients.

As yet another example, the base 3212 may include a processor 3237. Even if the shoe 3210 includes a processor 3236, it may be useful to provide supplemental processing capacity within the base 3212. For example, the shoe processor 3236 may be sized so as to support minimal operation of the shoe 3210 but not all aspects of operation, such as complex data processing (e.g., identifying signatures); more data intensive tasks may be performed by a processor 3237 in the base 3212.

Similarly, even if the shoe 3210 includes a data store 3238, the base 3212 likewise may include a data store 3239. As noted with regard to certain other features in the base 3212, the base data store 3239 may be more capable then the shoe data store 3238. However, again as with other features in the base 3212, the base data store 3239 may be of comparable capability (or even of lesser capability), e.g., the base data store 3239 and the shoe data store 3238 may have similar capacities and bandwidths but provide redundancy of data storage. In such instance, each data store 3238 and 3239 may serve as partial or complete backups of the other.

Other elements likewise may be utilized, whether in a base 3212 or otherwise.

Referring now to FIG. 33, a range of configurations for engaging a shoe with a foot of a container have been presented as examples. However, it may also be useful to consider not merely the mechanism for engaging a shoe with a foot, but also other factors relating to engagement. For example, it may be useful for a shoe to engage in only one, or in only a few, configurations. As a more concrete example, if electrical contacts are present on the shoe and foot (e.g., data and/or power ports as previously referenced), it may be useful to assure that the shoe engages such that contacts on the shoe are aligned with (and can make electrical contact with) contacts on the foot. FIG. 33 shows three top-down views of various shoes, illustrating arrangements wherein a shoe may engage a foot in substantially only one configuration.

In FIG. 33, a top-down view of shoe 3310A is shown. A shoe coupling 3311A is shown, in the form of an upward-extending projection. A similar arrangement may be seen in cross-section in FIG. 31. As may be understood, the shape of the projection—an isosceles triangle—is such that the projection would fit into a correspondingly-shaped socket in only one orientation.

FIG. 33 also shows a top-down view of shoe 3310B. Shoe couplings 3311B are shown, in the form of magnets. A similar arrangement may be seen in cross-section in FIG. 26. Given the arrangement of the magnets, the opposing magnets in the foot (as shown in FIG. 26) would only cooperate with the magnets in the shoe in a single configuration. If the shoe were rotated out of alignment, for example, the magnets would not engage (although with sufficient strength, the magnets may actually rotate the shoe back into the proper configuration for engagement). It is noted that in principle, it may be possible to link the magnets in shoe couplings 3311B in a clearly incorrect manner; for example, if the shoe were rotated 180 degrees out of correct alignment, and then offset sideways, the magnets may engage. However, firstly such a problem could be addressed by selecting the polarity of the magnets so that this is not possible (e.g., the shoe's upper magnet is north-up, while the shoe's lower magnet is south-up, in which case rotating the shoe would cause the magnets on shoe and foot to repel rather than engage). Secondly, such misorientation and misalignment may be considered so clearly visible that users would not be expected to mistake such errors for a reasonable engagement.

FIG. 33 also shows a top-down view of shoe 3310C. Shoe couplings 3311C are again shown, in the form of loops. A similar arrangement may be seen in cross-section in FIG. 25. Given the arrangement of the loops, if the shoe were misoriented and/or misaligned the loops would not fit over the corresponding pins on the foot (shown in FIG. 25). Thus, the shoe 3310C could engage a corresponding foot in substantially only a single configuration.

It is noted that engaging in substantially a single configuration does not require an absolute or perfect orientation, or an utter impossibility of any other arrangement. For example, as noted with regard to shoe 3310B in FIG. 33, an arrangement where errors may be considered clearly unreasonable may still be suitable. In addition, with regard to imperfect alignment, it is noted that real-world structures such as electrical contacts typically have some diameter, and thus some degree of “slip” in aligning shoe and foot may be acceptable. So long as a shoe can only be readily engaged such that all such functional requirements are met—e.g., ports are aligned to as to make contact and transmit data, power, etc.—the quality of being aligned substantially only in one configuration may be satisfied. In colloquial terms, the intent is that the shoe “fits and works” in one way only, and cannot reasonably be put in backwards (for example).

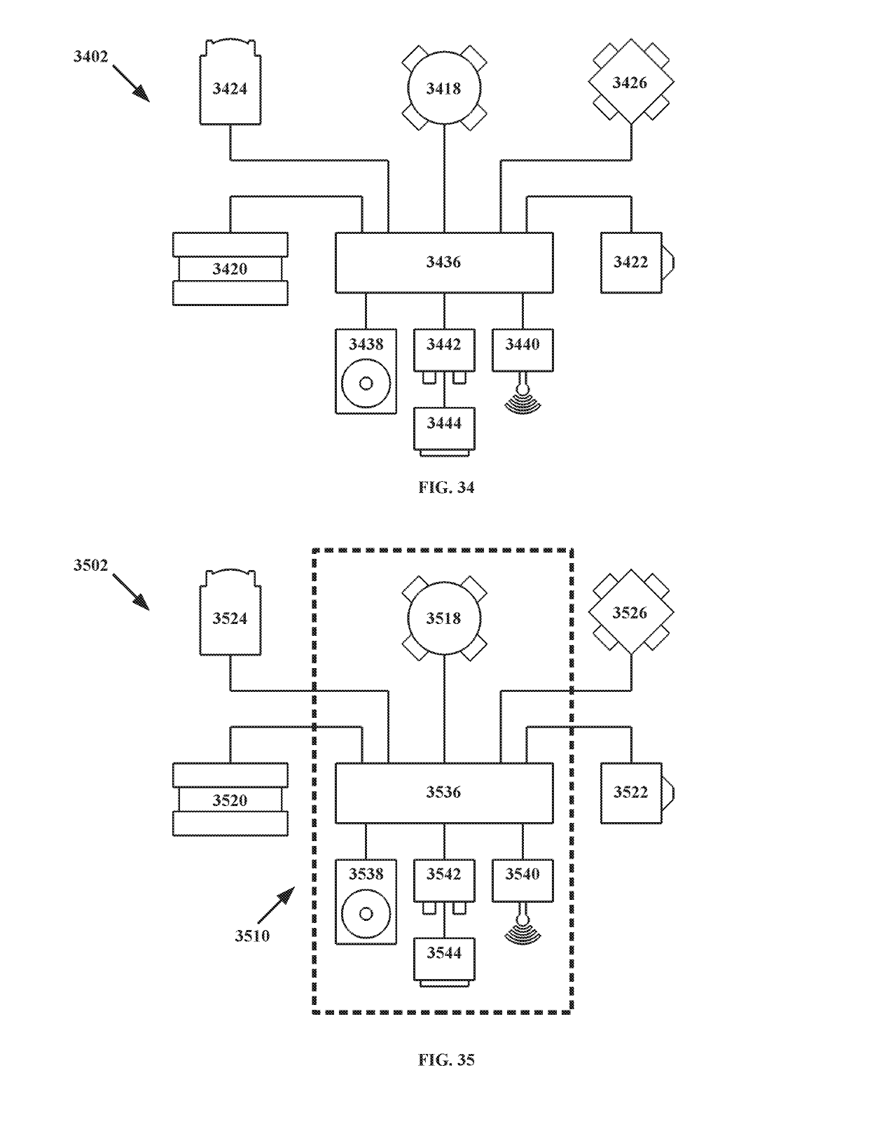

Now with reference to FIG. 34, an example of elements as previously described with regard to a container, shoe, etc., is presented in schematic form. Where certain previous illustrations omitted power connections, data connections, power sources, etc. for simplicity, FIG. 34 indicates such connections, etc. In the arrangement of FIG. 34, features an assembly for collecting and analyzing data are shown. The assembly collectively is identified as a container 3402. However, no distinction is made between whether elements in FIG. 34 are disposed on a container proper, on a shoe engaged with such a container, on a base, distal from the container in some other arrangement, etc.; elements as shown do not necessarily reflect physical configuration. For example, in certain embodiments every element shown in FIG. 34 as present with the container 3402 may be physically disposed on a shoe, while in other embodiments every element shown may be integrally incorporated into the container 3402 proper. Other arrangements also may be suitable.

FIG. 34 shows a processor 3436, as previously described. An orientation sensor 3418, pressure sensor 3420, cap sensor 3422, droplet sensor 3424, and quantity sensor 3426 are shown in communication with the processor 3436, e.g., so as to communicate power and/or data among the processor 3436 and sensors 3418 through 3426. As also previously indicated, the number, type, arrangement, etc. of the sensors 3418 through 3426 may vary considerably, and the arrangement in FIG. 34 is an example only. In addition, a data store 3438, communicator 3440, power source 3442, and charger 3444 are also shown. Again, the particular arrangements are an example only, and others may be suitable; for example, as shown the power source 3442 is in communication with the processor 3436 and most other elements shown are in communication with the power source 3442 indirectly via the processor 3436; such an arrangement is only one example, and should not be understood as limiting.

Turning to FIG. 35, as noted with regard to FIG. 34 the physical arrangement of elements therein may vary considerably, e.g., elements may be on a container proper, may be on a shoe, etc. FIG. 35 illustrates an example wherein such elements are so divided. The assembly as shown is associated with a container 3502; a portion of that assembly is in turn disposed on a shoe 3510, such as may be disposed physically on the foot of a container 3502. In the arrangement shown in FIG. 35, a processor 3536, data store 3538, communicator 3540, power supply 3542, charger 3544, and an orientation sensor 3518 are disposed on the shoe 3510. Additional elements, namely a pressure sensor 3520, cap sensor 3522, droplet sensor 3524, and quantity sensor 3526 are disposed elsewhere than the shoe 3510, for example being disposed at various points on the container 3502 (e.g., the droplet sensor 3524 at the nozzle of the container 3502, as shown in certain previous illustrations). In terms of schematics and function, the distinction between whether a given sensor is on the shoe 3510 or directly on the container 3502 may be at least largely a logical matter; in terms of connections, etc., the cap sensor 3522 (for example) could be within the dashed line identifying the shoe 3510, but may not necessarily function differently in such instance. As noted previously, various sensors and/or other elements may be disposed in various locations on/with respect to a container; while the arrangement in FIG. 35 provides an example, this example should not be understood as limiting.

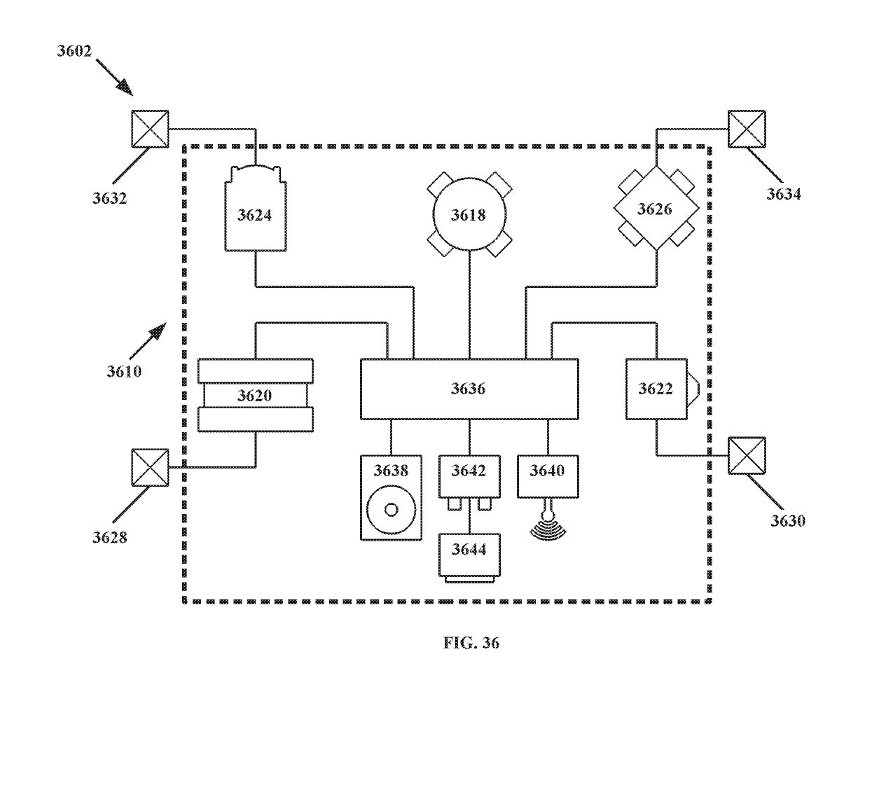

Turning to FIG. 36, as described previously (e.g., with regard to FIG. 15) some portion of a given sensor may be disposed on a shoe, while some other portion of that sensor such as a probe for the sensor may be disposed elsewhere, such as on the container proper (as opposed to the shoe). Such an arrangement is illustrated in FIG. 36. Therein a container 3602 is shown, with a portion thereof identified as a shoe 3610. A processor 3636, data store 3638, communicator 3640, power supply 3642, charger 3644, orientation sensor 3618, pressure sensor 3620, cap sensor 3622, droplet sensor 3624, and quantity sensor 3626 are disposed on the shoe 3610. A pressure probe 3628, cap probe 3630, droplet probe 3632, and quantity probe 3634 are disposed outside the shoe 3610, for example wrapped around the bottle, at the seat for the cap, at the nozzle, and in the bottom of the container respectively (e.g., as shown in FIG. 15, though this is an example and is not limiting). The pressure probe 3628, cap probe 3630, droplet probe 3632, and quantity probe 3634 are in communication with the pressure sensor 3620, cap sensor 3622, droplet sensor 3624, and quantity sensor 3626 respectively as shown. While in FIG. 36 the probes 3628 through 3634 are shown distinct from the corresponding sensors 3620 through 3626 so as to indicate relative placement with respect to the container 3602, in other instances it may be equally correct to refer to the probes 3628 through 3634 as being part of their corresponding sensors 3620 through 3626 respectively. Referring to an element as part of a sensor, as opposed to a probe for a sensor, may to at least some degree be a matter of language; the sensor and/or larger apparatus may operate similarly regardless.

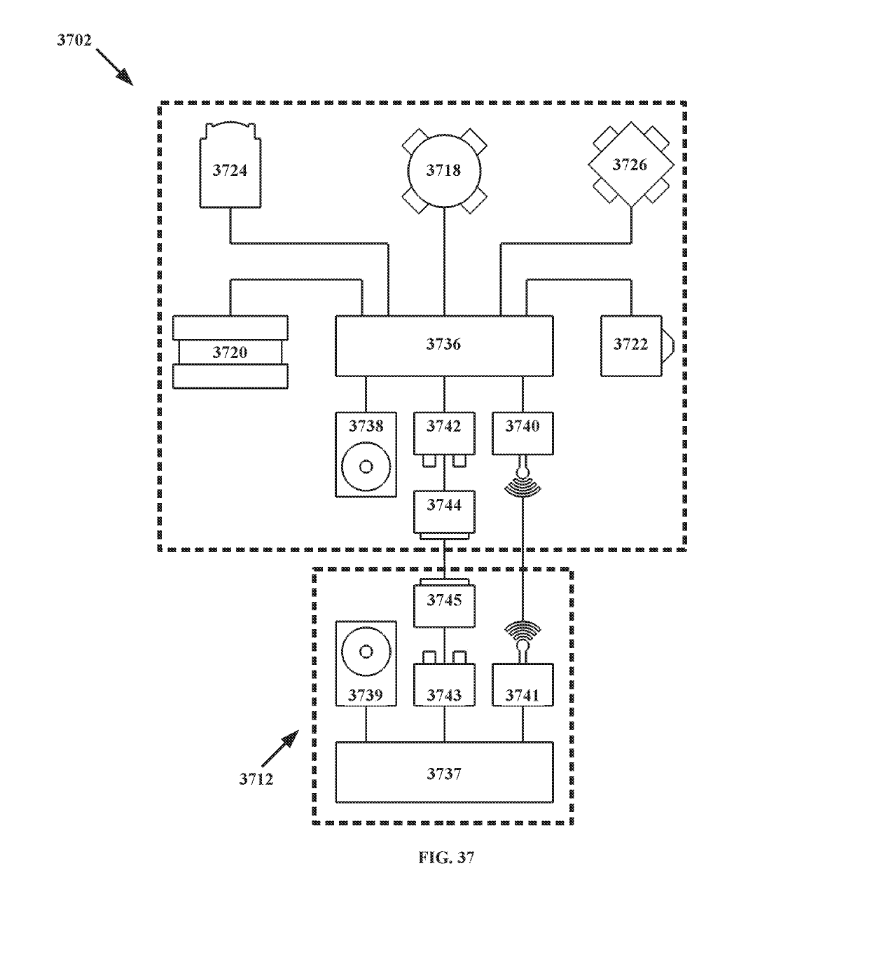

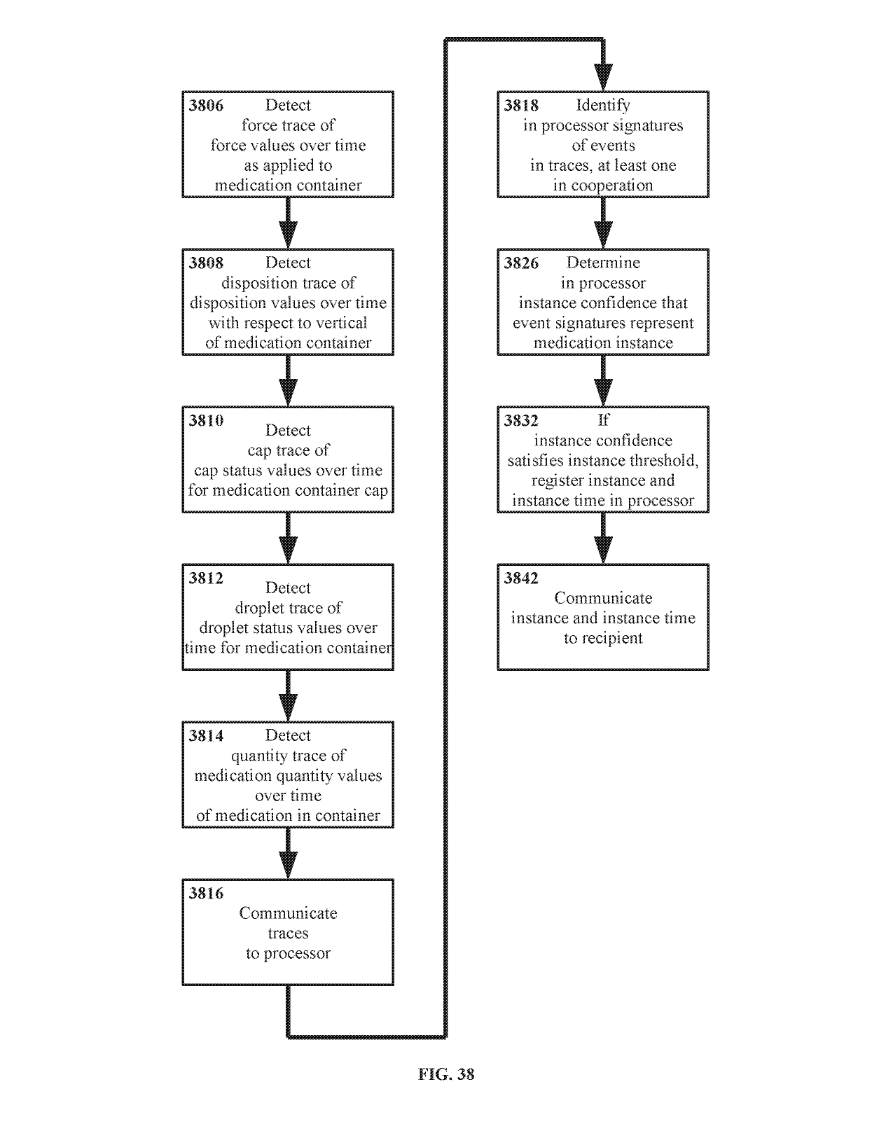

Now with reference to FIG. 37, as noted other elements besides a container (and associated shoe, annex, etc.) may be used in association with a smart container. For example, a base may be so used. The arrangement in FIG. 37 shows a container 3702 and a base 3712. (As with FIG. 34, no distinction is made between whether elements on the container are on a shoe, an annex, etc., rather a distinction is made between the container 3702 and the base 3712 for explanatory purposes.) As may be seen, the container 3702 includes a processor 3736, data store 3738, communicator 3740, power supply 3742, charger 3744, orientation sensor 3718, pressure sensor 3720, cap sensor 3722, droplet sensor 3724, and quantity sensor 3726. The base includes a base processor 3637, base data store 3639, base communicator 3641, base power supply 3643, and base charger 3645. (No sensors are shown on the base in the example of FIG. 37, but the presence of sensors and/or other elements is not excluded.)

As may be seen, the charger 3744 in the container 3702 is shown to be in communication with the base charger 3745 in the base 3712. Thus, in at least certain instances (e.g., when the container 3702 is sitting on the base 3712) power may be transferred from the base 3712 to the container 3702 (and/or vice versa). Likewise, the communicator 3740 in the container 3702 is shown to be in communication with the base communicator 3741 in the base 3712, thus in at least certain instances data may be transferred from the base 3712 to the container 3702 (and/or vice versa). (It is noted that although the communicator 3740 and base communicator 3741 are illustrated as using waves, such as Wi-Fi communicators, this is an example only and is not limiting. While wireless communication may be useful for certain embodiments, for example in enabling communication between container 3702 and base 3712 even when the container 3702 is not on the base 3712, wired or other contact communicators may be equally suitable.)

With reference now collectively to FIG. 38 through FIG. 47, previously an example arrangement for acquiring medication data and applying that data with regard to adherence is shown in FIG. 3. However, many variations may be suitable, including but not limited to those shown in FIG. 38 through FIG. 47.

For example, with reference now specifically to FIG. 38, as has been noted previously embodiments are not limited only to pressure and orientation sensing, values, and/or traces, nor to specifically two such properties. Thus, in FIG. 38 an example arrangement is shown that includes detecting 3806 a force trace of force values as experienced by a medication container. Such detection may be similar to detecting pressure as referred to in certain previous examples, in that applying a squeezing pressure to an eyedrop bottle or similar may be considered a special case of forces in general being experienced by a medication container. However, while the forces detected 3806 in FIG. 38 may be such a squeezing pressure, other forces—whether user-applied such as a force applied to depress a plunger of a syringe, or not user-applied such as the force due to gravity on a container at rest—may be sensed, and traces produced therefrom.

Similarly, a disposition trace of disposition values for the medication container is also detected 3808. While previous examples may have referenced orientation, e.g., the rotation of a container around some axis or axes, other aspects of disposition may be detected and/or considered. For example, the position of a container in space (as distinct from the orientation of that container), the velocity or acceleration of the container, the distance of the container from some reference point (such as on a base unit for the container), some combination thereof, etc. may be detected in detecting disposition 3808.

A cap trace of cap trace values is detected 3810. Such a trace may or may not be numerical (as may other traces). For example, a cap trace may include a numerical measurement of pressure applied to a sensor by a cap or a numerical measurement of light levels reaching a sensor (where light levels may be lower when the cap is in place and blocking light from reaching such a sensor). Alternately, a cap trace may be non-numerical, such as a binary determination of whether a circuit is connected (e.g., if a cap includes a conductive path that connects two contacts on a container when the cap is in place). Other arrangements also may be suitable.

A droplet trace of droplet values is detected 3812. For example, such a trace may indicate whether a droplet is present at the end of a nozzle at any moment in time, the motion of a droplet through a nozzle, etc.

A quantity trace of medication quantity values is detected 3814. For example, the trace may indicate a quantity in volume (e.g., milliliters), in mass (e.g., grams), or in distinct units (e.g., a measured or estimated number of tablets or droplets remaining). A quantity trace alternately may address quantity in an indirect and/or abstract manner (as may other traces); for example, a quantity trace may refer to how many days of medication remain, etc.

The several traces (in the example of FIG. 38, force, disposition, cap status, droplet, and medication quantity traces) are communicated 3816 to a processor.

In the processor, one or more signatures are identified 3818 within the traces, with each signature being consistent with some corresponding event associated with an instance of administering a medication to a subject. At least one such signature is identified 3818 through consideration of two or more of the several traces in cooperation. For example in the arrangement of FIG. 38, force and disposition, force and droplet, cap status and droplet and quantity, etc.

Continuing in FIG. 38, a determination is made 3826 in the processor as to a medication instance confidence, indicating a degree to which the signatures may indicate that medication has been administered. If the instance confidence is determined to meet an instance threshold therefor, an instance of medication administration and an instance time for that medication instance is registered 3832 in the processor. The medication instance and medication instance time are also communicated 3842 to a recipient.

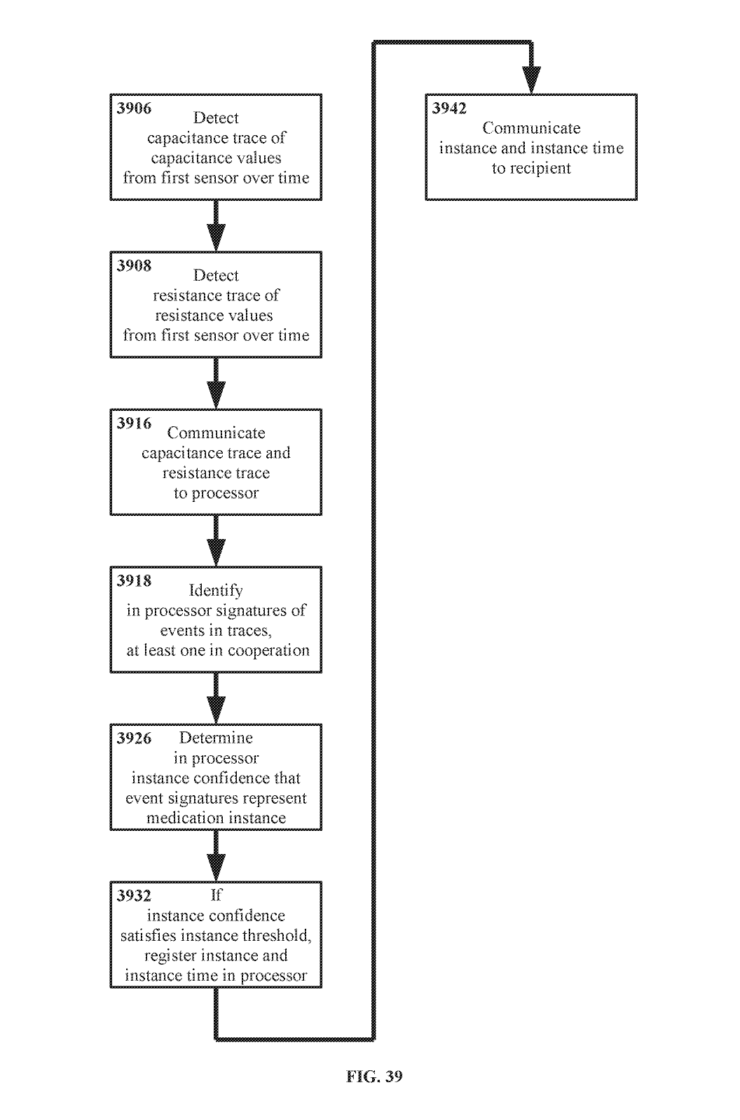

Now with reference to FIG. 39, as has been noted multiple traces and/or traces addressing different properties may be acquired from a single sensor. For example, in FIG. 39 a capacitance trace of capacitance values from a first sensor are detected 3906 over time, and a resistance trace of resistance values from that first sensor are also detected 3908 over time. Such an arrangement may occur when utilizing certain types of sensor. For example, certain sensors for detecting and/or measuring pressure applied to a surface and/or distortion of that surface may include a “sandwich” of a dielectric layer between two conductive layers. As pressure is applied and/or as the sandwich is distorted, the resistance of one or both conductive layers and/or the resistance through the sandwich as a whole may vary; likewise, the capacitance of the sandwich also may vary. In certain instances, analysis of both resistance and capacitance may be more illuminating than analysis of resistance or capacitance alone. For example, broad-area pressure from fingertips may exhibit different characteristic signatures in capacitance, resistance, or both than point pressure from contact with another object in a purse, pocket, etc. Other events also may be identified and/or distinguished from one another by evaluating both resistance and capacitance (whether addressed individually or in cooperation), even when such events may not be identified and/or distinguished by evaluating a single value for pressure as a whole.

Continuing in FIG. 39, the capacitance and resistance traces are communicated 3916 to a processor. In the processor, signatures are identified 3918 within the traces consistent with corresponding events associated with a medication instance. At least one signature is identified 3918 through consideration of both the capacitance and resistance traces in cooperation.

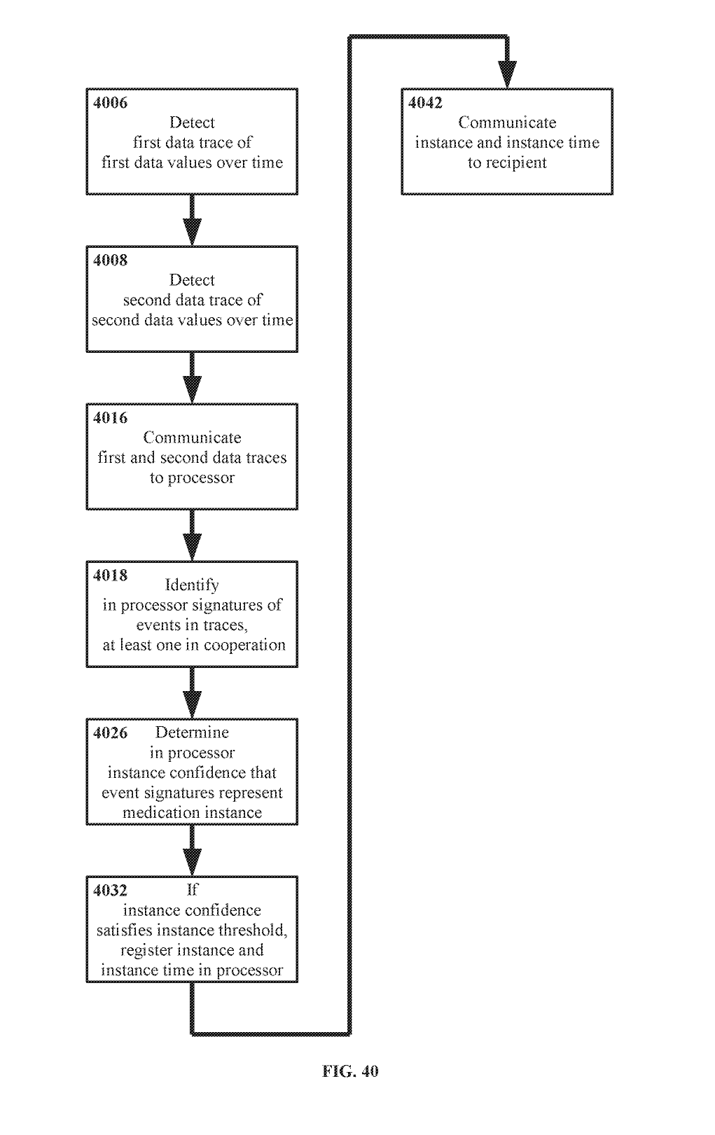

A determination is made 3926 in the processor as to a medication instance confidence, and if the instance confidence is determined to meet an instance threshold therefor an instance of medication administration and an instance time are registered 3932 in the processor. The medication instance and medication instance time are communicated 3942 to a recipient.

Where FIG. 39 showed the use of several traces, and FIG. 39 showed two traces as may be acquired from a single sensor, as shown in FIG. 40 traces may vary even more broadly, to the point that any information that may be usefully acquired and considered, regardless of source or content, may be suitable for certain embodiments. In addition, while certain examples herein have referred to medication events—e.g., squeezing out an eyedrop into an eye, removing a pill from a bottle, or otherwise performing some specific action that may be referred to colloquially as “taking the medication”—it is emphasized that contextual events likewise may be considered, along with signatures therefor. For example, as previously shown, removing a cap—while perhaps not literally corresponding to taking a medication—may be understood as helping to establish the context for taking a medication. Thus, even if the act of taking the medication itself is not (or even could not be) identified directly, nevertheless it may be determined with confidence that the medication has been taken by considering events as may take place in a context of an instance of taking the medication (and the signatures of such contextual events).

In FIG. 40 a first data trace of first data values over time are detected 4006, and a second data trace of second data values over time also are detected 4008. The type of data, the sources of data, etc., are not limited and may vary considerably. The first and second data traces are communicated 4016 to a processor. In the processor, signatures are identified 4018 within the traces consistent with corresponding events associated with a medication instance. The events also are not limited, and in particular are not limited only to the act of taking the medication itself; contextual events (and signatures thereof) may be suitable. At least one signature for at least one such event is identified 4018 through consideration of both the first and second data traces in cooperation. (However, in certain embodiments even such cooperation itself may be optional; for certain embodiments it may be suitable to identify signatures in only a single data trace, and/or to acquire two or more data traces without identifying signatures in both traces in cooperation.)

A determination is made 4026 in the processor as to a medication instance confidence, and if the instance confidence is determined to meet an instance threshold therefor an instance of medication administration and an instance time are registered 4032 in the processor. The medication instance and medication instance time are communicated 4042 to a recipient.

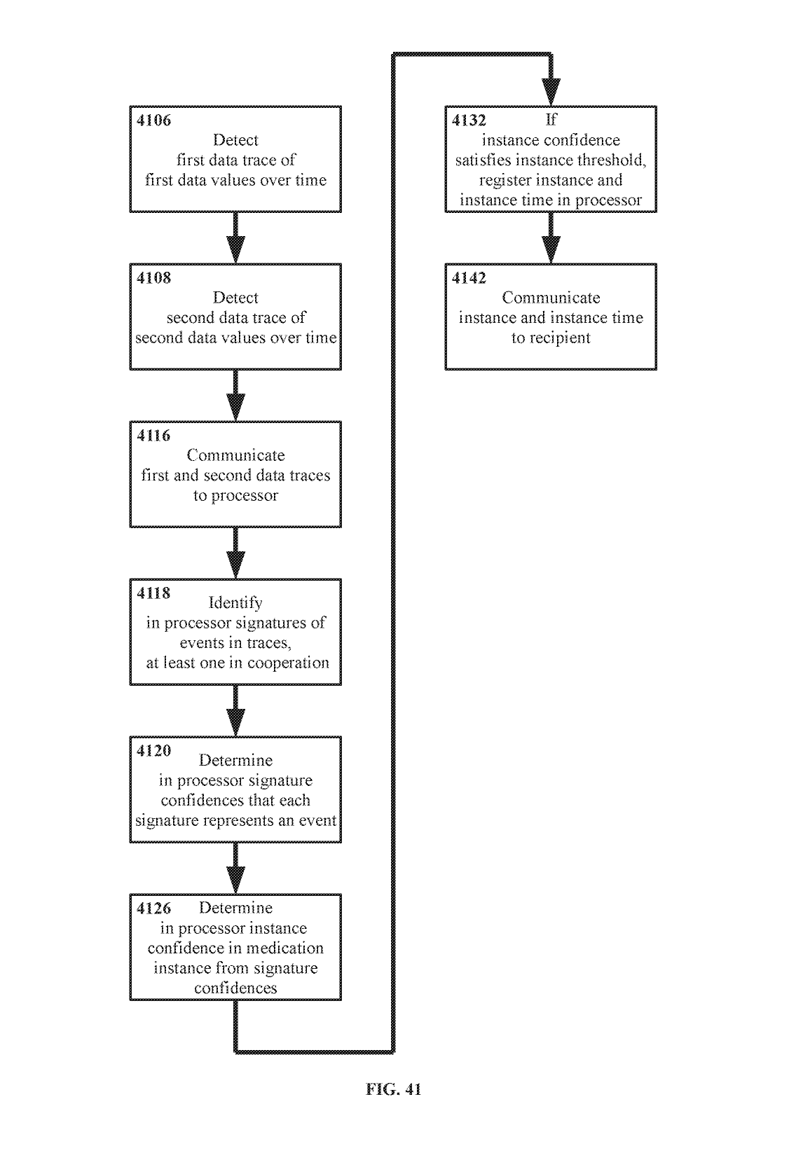

With regard now to FIG. 41, it may be suitable for some embodiments to determine an overall confidence that a medication has been taken in a particular situation—the instance confidence—in whole or in part based on a confidence that one or more signatures represents a real-world event.

For example, a first data trace of first data values over time may be detected 4106, and a second data trace of second data values over time also likewise detected 4108. The first and second data traces are communicated 4116 to a processor. In the processor, signatures are identified 4118 within the traces consistent with corresponding events associated with a medication instance, with at least one signature for at least one such event identified 4118 through consideration of the first and second data traces in cooperation.

Moving on in FIG. 41, one or more of the signatures may be evaluated 4120 to determine a confidence that the trace thereof does indicate that a corresponding event has happened. For example, the shape of a trace may be compared with a characteristic shape for a trace expected to occur when a container is shaken to mix a medication therein. Rather than necessarily identifying signatures with a simple yes/no, some degree of match may be determined, e.g., a 90% match, a “high confidence match”, etc. In considering shape, for example, the geometry of the trace could be compared mathematically to some reference standard for a “shake bottle” signature, to determine to what degree (if any) the actual trace matches the standard therefor. Not all signatures necessarily must be evaluated 4120 with regard to signature confidence, and not all embodiments necessarily must evaluate 4120 signature confidence at all.

However, where signature confidence is determined 4120, the instance confidence—the overall confidence that a medication has been taken—may be determined 4126 at least in part based on the signature confidences. For example, if shaking, cap removal, droplet squeezing, and cap replacement signatures are present, then the confidence level that those various signatures represent real events of shaking a medication container, opening the cap, squeezing out a droplet, and replacing the cap may be considered in determining 4126 the overall instance confidence that the medication has been taken. However, other factors such as ordering of signatures, etc., whether related individual signature confidence or not, may be considered in addition or instead.

Still with reference to FIG. 41, and regardless of how the instance confidence is determined 4126, if the instance confidence is determined to meet an instance standard therefor an instance of medication administration and an instance time are registered 4132 in the processor.

It is noted, particularly though not exclusively with regard to arrangements wherein determining instance confidence considers signature confidences that the term “threshold” for instance confidence should be understood broadly. A threshold does not imply a simple numerical minimum. Rather, some standard and/or standards may define what a particular embodiment may treat as “a good enough match”. Such standards may be complex, may change dynamically, etc., and are not limited only to a simple minimum value. Thus, while the threshold for instance confidence may be a mathematical minimum such as “at least 90% confidence”, this is not required (in particular since confidence will not necessarily be determined in such mathematical terms for all embodiments).

Continuing in FIG. 41, the medication instance and medication instance time are communicated 4142 to a recipient.

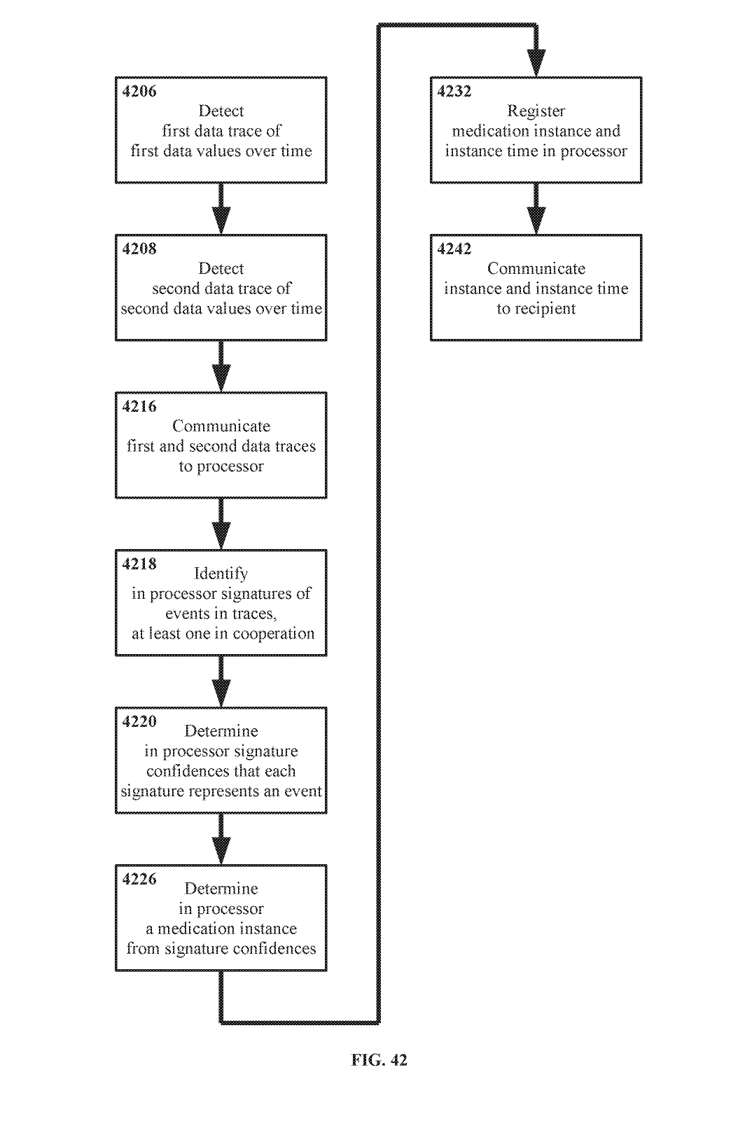

With reference now to FIG. 42, while confidence determination may be useful in some embodiments, determining the instance confidence and/or signature confidence is not necessarily required for all embodiments. For example, a first data trace of first data values over time may be detected 4206, and a second data trace of second data values over time detected 4208. The first and second data traces are communicated 4216 to a processor. In the processor, signatures are identified 4218 within the traces consistent with corresponding events associated with a medication instance, with at least one signature for at least one such event identified 4218 through consideration of the first and second data traces in cooperation.

While in certain previous examples a confidence has been determined as to whether signatures suggest medication has been taken, in the example of FIG. 42 it may be suitable to make a definitive yes/no determination 4226 as to whether a medication instance has taken place. Determining confidence, either for individual signatures or for a medication instance overall, may not be necessary. Rather, when present the medication instance and instance time are registered 4232 in the processor. The medication instance and medication instance time are also communicated 4242 to a recipient.

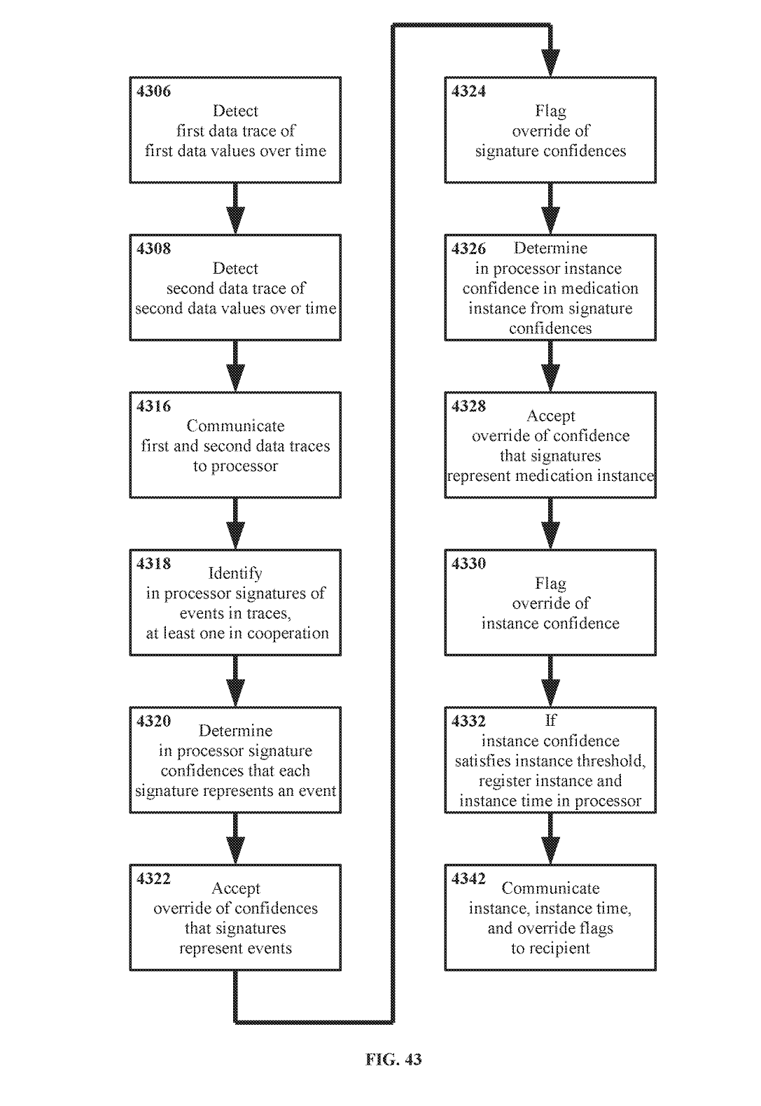

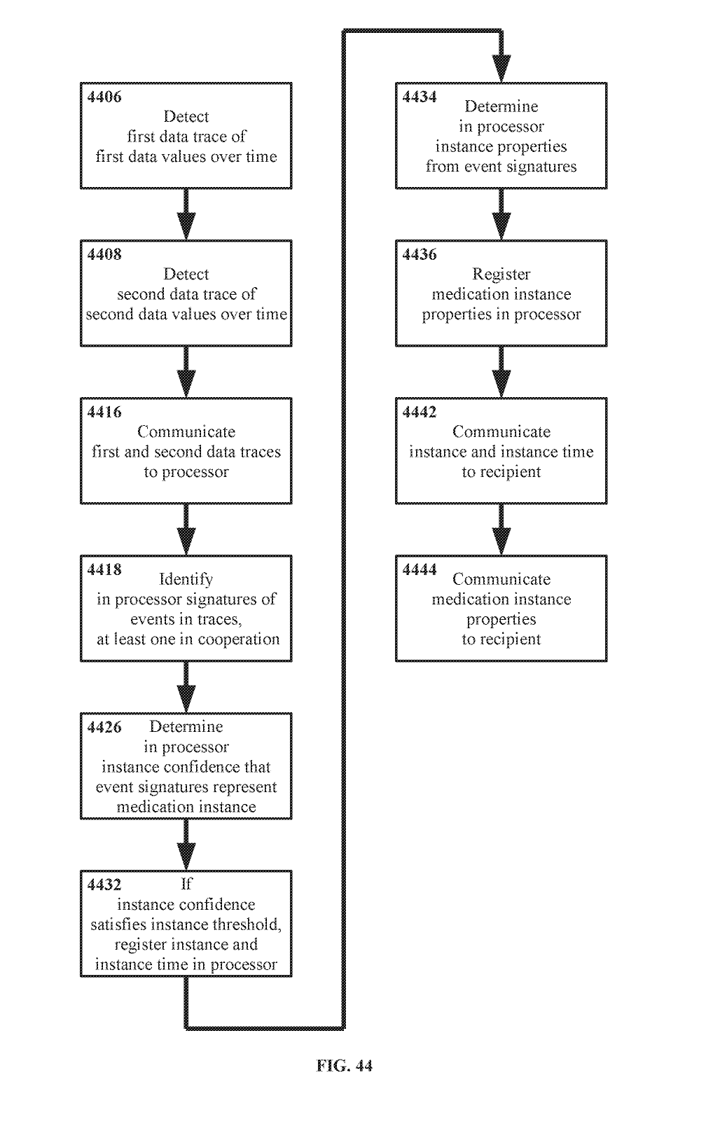

Now with reference to FIG. 43, in certain embodiments it may be useful to intervene to identify traces, signatures, etc. as reflecting a medication instance even if a medication instance otherwise may be determined as having low or even no confidence.

For example, a first data trace of first data values over time may be detected 4306, and a second data trace of second data values over time detected 4308. The first and second data traces are communicated 4316 to a processor. In the processor, signatures are identified 4318 within the traces consistent with corresponding events associated with a medication instance, with at least one signature for at least one such event identified 4318 through consideration of the first and second data traces in cooperation.

Signature confidences may be determined 4320 for one, some, or all signatures (or potential signatures). However, regardless of whether a given trace is determined to exhibit a particular signature at a particular point, the confidence determination 4320 may be overridden 4322. That is, a trace otherwise determined to exhibit a signature may be overridden so as to be treated as though no such trace had been determined to be present, and/or to be treated as though a different signature had been determined to be present; and/or a trace otherwise determined not to exhibit a signature may be overridden so as to be treated as though a trace had been determined to be present. Typically, though not necessarily, such overrides may be manual, wherein the medication user or some other person intercedes to indicate that in fact some event has taken place, even if evaluation of the traces may not clearly identify signatures for that event. Such an override may serve to avoid errors. In addition, overrides may serve as training; not all users may manipulate a container in the same manner when dispensing medication, for example, thus the traces during various events may appear different for different users. A user overriding determinations as to whether signatures are present may enable improvement and/or correction of signature definitions, determination processes for evaluating signatures, etc.

In addition, while as noted such overrides may be performed by persons taking the medication and/or other persons, it may also be useful for certain other entities to have at least some degree of override authority in addition or instead. For example, while a given processor may have ready access only to a relative small data set in evaluating traces for signatures (e.g., some “basic” set of common signature definitions), a questionable case may be submitted for automatic review with access to a larger database (e.g., a large library of real-life signatures for many different individuals). In such instance, some oversight processor and/or other system may be granted some degree of override authority.

Continuing in FIG. 43, when overrides of signature confidences take place, those overrides may be flagged 4324 in some manner so as to identify which signatures were overridden, in what manner (changed to a different signature, deleted, added where not originally present, etc.), by whom, etc. However, while such flagging may be present, not all embodiments with override capability necessarily will or must include such flagging of overrides.

A determination is made 4326 of medication instance confidence. For embodiments wherein signature confidences are determined (such as in FIG. 43), such determination 4326 may be made at least in part based on signature confidences.

As with the signature confidence determinations, the instance confidence determination 4326 may be overridden 4328. Thus, medication instances that are detected may be overridden as not being present, medication instances that are not detected may be overridden as being present, etc. Persons, automated systems, etc. again may have varying degrees of override authority in various embodiments. As with signature overrides, advantages such as error correction, system learning, etc. may be facilitated by medication instance overrides. Also, as with signature overrides, overrides of instance confidence may be flagged 4330.

While the arrangement in FIG. 43 shows both signature and medication instance overrides, it is not required that every embodiment with one such capability include both. Signature overrides may be present without medication instance overrides, and medication instance overrides may be present without signature overrides. Also, medication instance overrides may be present even if signature confidences themselves are not determined, etc.

Moving on in FIG. 43, if the medication instance confidence satisfies a threshold therefor, the medication instance and medication instance time are registered 4332 in the processor. Registration 4332 may include registering some or all override flags (though this is not required). The medication instance and medication instance time are also communicated 4342 to a recipient. Communication 4342 also may include communicating some or all override flags (though again this is not required).

Now with reference to FIG. 44, in certain previous examples only a medication instance and a medication instance time thereof are indicated as being registered and communicated to a recipient. However, such information is not limited, and other information may be registered and/or communicated likewise. For example, as noted with regard to FIG. 43 data regarding whether overrides took place (e.g., flags) may be registered and or communicated.

In FIG. 44, a first data trace of first data values over time is detected 4406, and a second data trace of second data values over time is detected 4408. The first and second data traces are communicated 4416 to a processor. In the processor, signatures are identified 4418 within the traces consistent with corresponding events associated with a medication instance, with at least one signature for at least one such event identified 4418 through consideration of the first and second data traces in cooperation.

A medication instance confidence is determined 4426, and the instance and instance time are registered if that instance confidence is determined 4432 to meet a threshold therefor.

Additional information may be determined, and or registered, communicated, etc. For example, various properties of the medication instance may be determined 4434 in the processor from the event signatures. As a more concrete example, the dosage of medication dispensed may be determined. In comparing FIG. 4 with FIG. 7, it may be seen that the traces in FIG. 4 exhibit signatures for two droplets of medication, while the traces in FIG. 7 exhibit signatures for three droplets. As another example, the time interval between doses may be determined, based on the instance times of two consecutive medication instances. Intervals may be of interest for example in that a user who takes one day's dose at 10 PM and the next day's dose at 9 AM may not be meeting the intent of a “once per day” medication regimen, even if meeting the letter. A variety of other information, including but not limited to whether the user is properly shaking a medication before administration, whether a user is storing a medication in a cool place (e.g., for certain medications as may be degrade at high temperatures), etc., may be determined in various embodiments, based at least in part on the types of data traces under consideration in those embodiments.

Continuing in FIG. 44, the medication instance and instance time are registered 4432 in the processor. When present, medication instance properties also may be registered 4336 in the processor (though this is not required). The medication instance and medication instance time are communicated 4242 to a recipient, and likewise the medication instance properties also may be communicated 4244 to the recipient (or to a different recipient), though again such is not required.

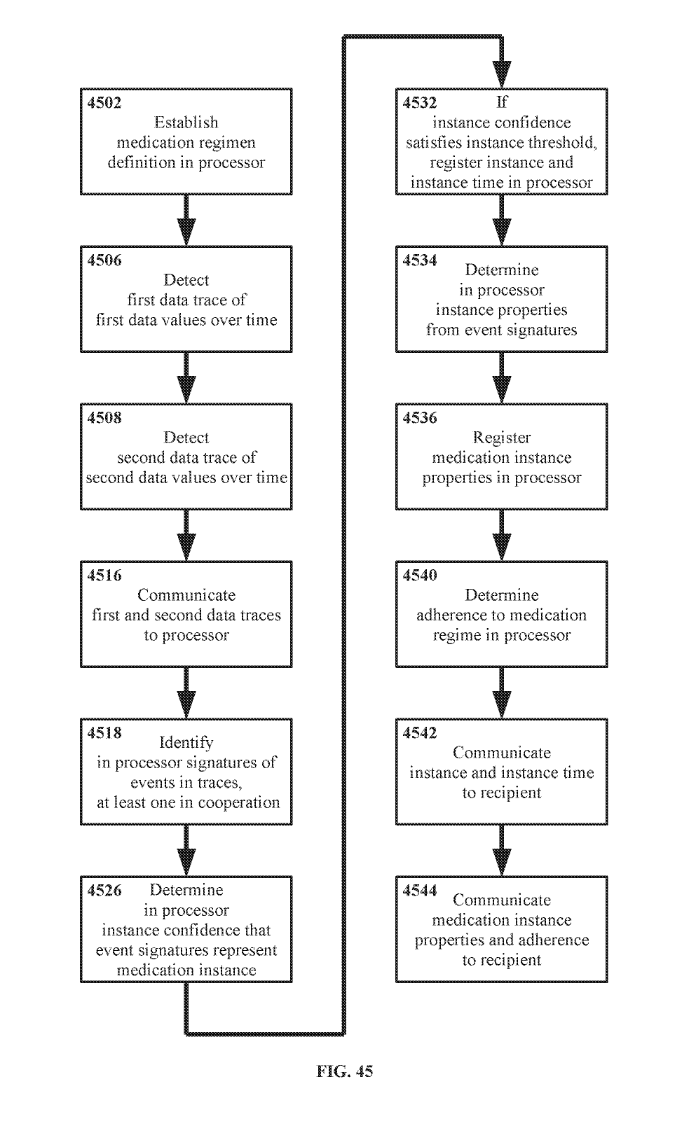

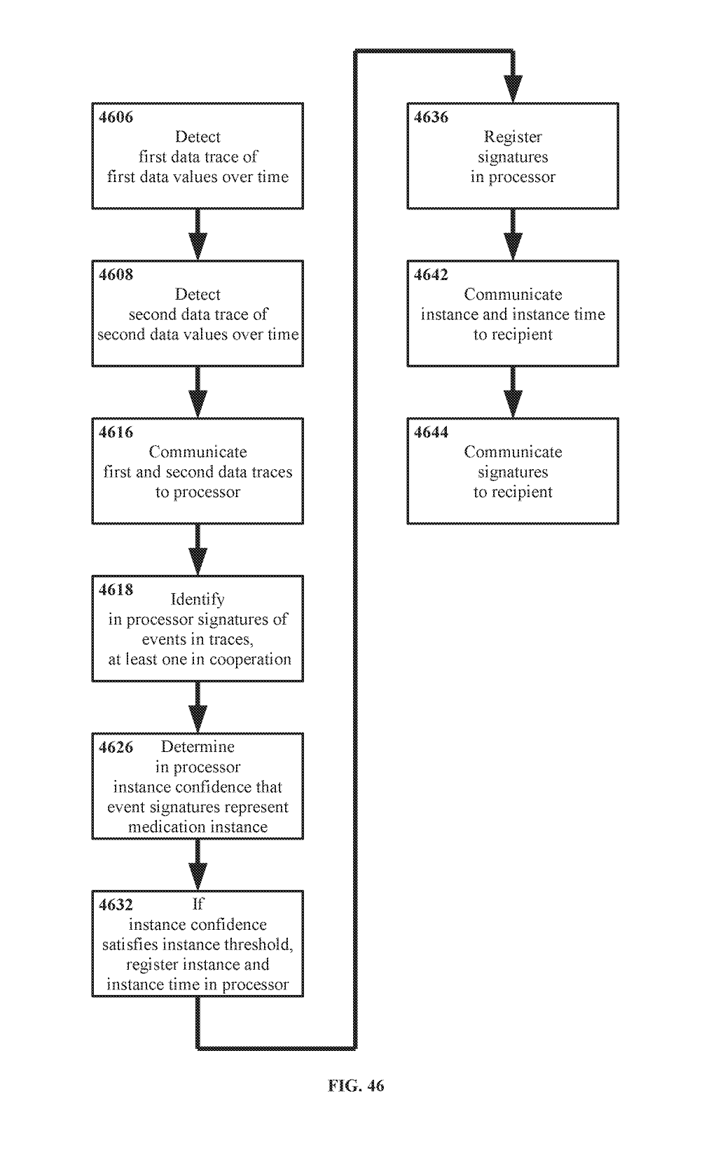

Now with reference to FIG. 45, as an additional example of information as may be determined from traces and/or signatures, it is noted that overall adherence to the a medication regime may be evaluated. In a processor, a medication regimen definition is established 4502. The particulars of such a regimen definition may vary, but may for example include the times at which a medication is to be taken, the number of times per day, the dosage, and so forth. Typically, though not necessarily such a definition may be instantiated on a processor in the form of executable instructions and/or digital information, though this is not limiting.

A first data trace is detected 4506, and a second data trace is detected 4508. The first and second data traces are communicated 4516 to a processor. In the processor, signatures are identified 4518 within the traces consistent with corresponding events associated with a medication instance, with at least one signature for at least one such event identified 4518 through consideration of the first and second data traces in cooperation. A medication instance confidence is determined 4526, and the instance and instance time are registered if that instance confidence is determined 4532 to meet a threshold therefor.

Again, medication instance properties may be determined 4534, and may be registered 4536. As noted previously (e.g., with regard to FIG. 44), medication instance properties may include times at which a medication is taken, intervals between doses, dosages, and so forth. Such properties may be specified by the medication regimen (as established in 4502). Thus, the actual use of the medication (medication instance properties) may be compared against the specified use of the medication (medication regimen definition), and the adherence of the user to the medication regimen may be determined 4540. The adherence as determined may take a variety of forms, such as raw data (e.g., the time, interval, and dosage for each occasion that the medication is used) or data processed in some manner so as to provide an adherence factor (e.g., a calculation that the user is “highly adherent”, “class V adherent”, “98% adherent”, etc.). Adherence factors likewise may vary, for example some may be single-value (e.g., 95%) while others may be multi-dimensional (e.g., level 5 medication time, level 3 medication intervals, and level 4 medication dosage), etc. The manner of determination and/or form of adherence factors or other adherence information is not limited.

The medication instance and instance time are communicated 4542 to a recipient. The medication instance properties and/or the adherence information determined therefrom also may be communicated 4544 to the recipient. Communicating medication instance properties and adherence information may be independent; either one may be communicated, both, neither, parts of either, etc.

Now with reference to FIG. 46, “raw data” also may be registered and/or communicated. A first data trace is detected 4606, and a second data trace is detected 4608. The first and second data traces are communicated 4616 to a processor. In the processor, signatures are identified 4618 within the traces consistent with corresponding events associated with a medication instance, with at least one signature for at least one such event identified 4618 through consideration of the first and second data traces in cooperation. A medication instance confidence is determined 4626, and the instance and instance time are registered if that instance confidence is determined 4632 to meet a threshold therefor.

In addition, in certain embodiments the event signatures themselves may be registered 4636 in the processor, and/or the entire traces or portions thereof may be registered 4636 in the processor (whether or not the trace portions exhibit signatures). Such an arrangement may be understood as logging the “raw data”, that is, the sensor inputs themselves, or at least portions of those sensor inputs exhibiting features of interest (such as signatures). Other data also may be collected and/or registered; the data that may be collected and/or registered is not limited.

Still with reference to FIG. 46, the medication instance and instance time are communicated 4642 to some recipient. Likewise, the event signatures, entire traces, etc., also may be communicated 4644 to that recipient (and/or to some other recipient).

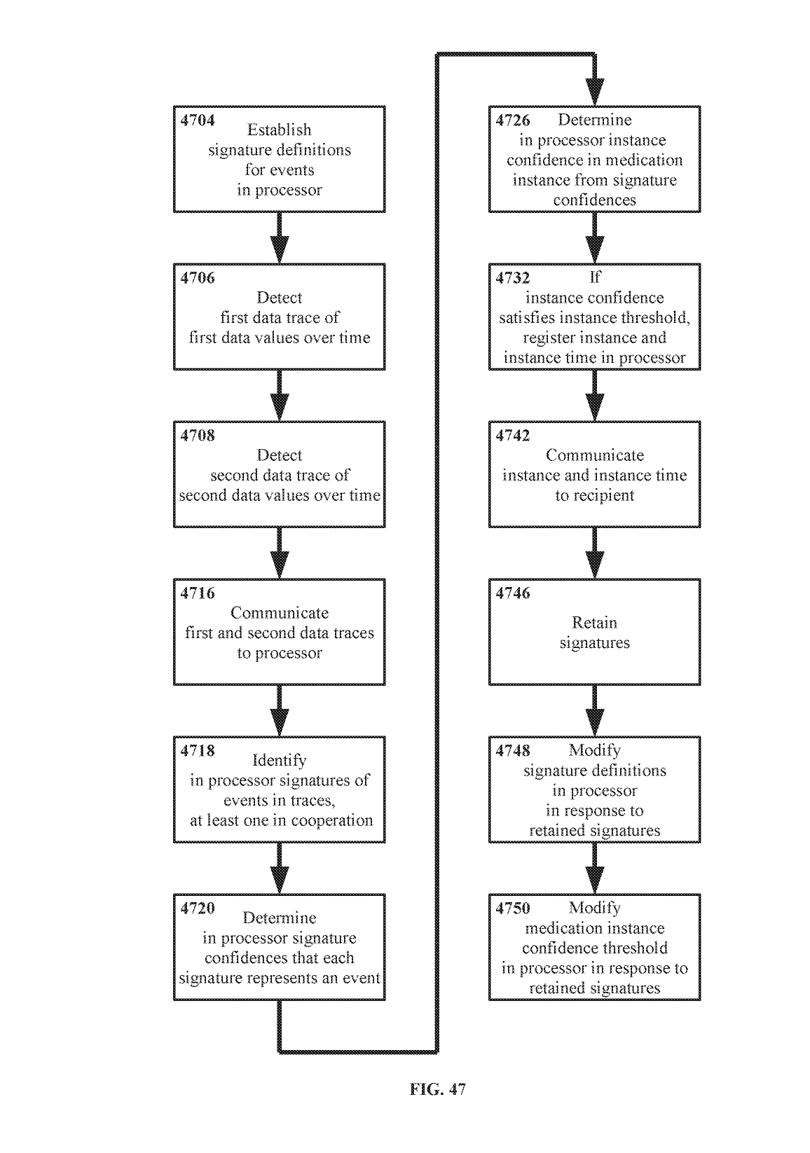

Moving on to FIG. 47, even if signatures and/or other data are not sent to an external recipient (e.g., being sent to an internal data store for storage and/or reference), signatures etc. nevertheless may be utilized further. In FIG. 47 signature definitions for one or more events are established 4704 in a processor. (Establishing such signature definitions is not exclusive to FIG. 47; other embodiments may likewise establish signature definitions, even if not stated explicitly in each case.) A first data trace is detected 4706, and a second data trace is detected 4708. The first and second data traces are communicated 4716 to a processor. In the processor, signatures are identified 4718 within the traces consistent with corresponding events associated with a medication instance, with at least one signature for at least one such event identified 4718 through consideration of the first and second data traces in cooperation.

In the example of FIG. 47, signature confidences are determined 4720 for some or all of the signatures as previously described. A medication instance confidence is determined 4726, and the instance and instance time are registered if that instance confidence is determined 4732 to meet a threshold therefor. The medication instance and instance time are communicated 4742 to some recipient.

However, where certain examples herein do not address further use of signatures, in the example of FIG. 47 signatures (and/or entire data traces, etc.,) are retained 4746. The manner by which signatures are retained 4746 is not limited; typically, though not necessarily such signatures may be recorded in a data store, though other arrangements may be suitable.

Continuing in FIG. 47, signature definitions (established in 4704) then may be modified 4748 in response to the retained signatures. Thus, the traces as received for one medication instance may inform detection of signatures for future medication instances. In more colloquial terms, the system may “learn” to better recognize signatures, based on the signatures that have already been detected. Such arrangements may for example facilitate personalization to a given user; if a user exhibits unusual trace forms (e.g., the shakiness as shown in FIG. 8) the system may adapt to more effectively identify signatures despite such unusual features. Alternately, if a user is deliberately deceiving the system (e.g., pretending to take a medication when they are not), the system may “learn” to detect such “spoofed” medication instances. The details of such improvement may vary depending on (among other factors) the particular traces, initial signature definitions, etc., and are not limited.

Similarly, the instance confidence threshold also may be modified 4750 in response to the retained signatures. For example, if a user habitually pauses for some time between opening a container cap and dispensing medication therefrom, that period of inaction could reduce confidence that a medication instance has occurred (e.g., if the original threshold were devised under the expectation that subjects would not wait in such fashion). However, by retaining signatures 4746 and modifying the instance confidence threshold 4750, such individual variations may be recognized and/or accommodated through continued use.

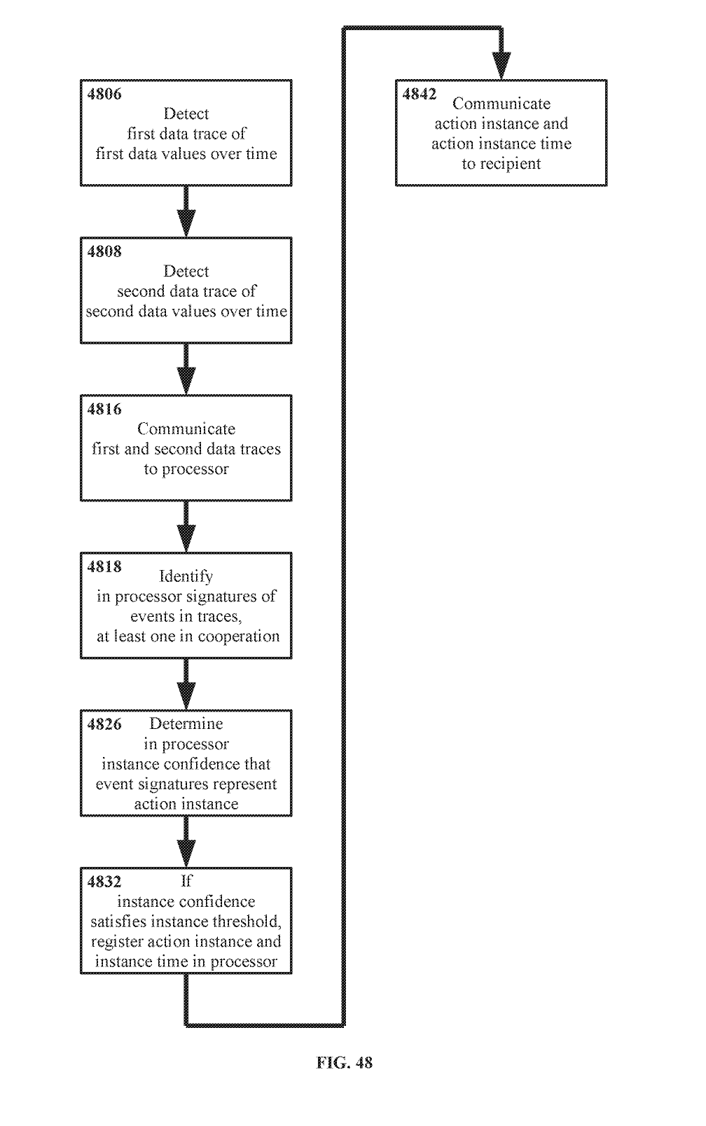

Now with reference to FIG. 48, for explanatory purposes certain examples herein have referred to specific applications, such as use of a medication, use of an eyedrop medication, specific sensors on a squeeze bottle of eyedrop medication, etc. However, it is emphasized that embodiments are not limited to eyedrop medication, to medication generally, to medical treatment as a whole, or to dispensing material from containers (whether for medical purposes or otherwise). FIG. 48 thus presents an arrangement not specific to medical purposes, dispensing medications, etc.

In the arrangement of FIG. 48, a first data trace is detected 4806, and a second data trace is detected 4808. The data traces, the manner in which the data traces are obtained, what features the data traces measure, and what actions those data traces may reflect, are not limited. The first and second data traces are communicated 4816 to a processor.

In the processor, signatures are identified 4818 within the traces consistent with corresponding events. At least one signature for at least one such event identified 4818 through consideration of the first and second data traces in cooperation. While the events under consideration may relate to use of a medication, this is not required. Alternate medical applications, such as use of a piece of therapeutic exercise equipment in addressing repetitive stress injuries, may be suitable for some embodiments. (It is noted that such embodiments would not necessarily address dispensing anything, medication or otherwise.) Likewise, non-medical applications also may be suitable.

Regardless of what phenomena may be of interest for a particular embodiment, an instance confidence is determined 4826 as to how likely it may be that the phenomenon in question has taken place. The instance and instance time are registered if that instance confidence is determined 4832 to meet a threshold therefor, and the instance and instance time are communicated 4842 to some recipient.

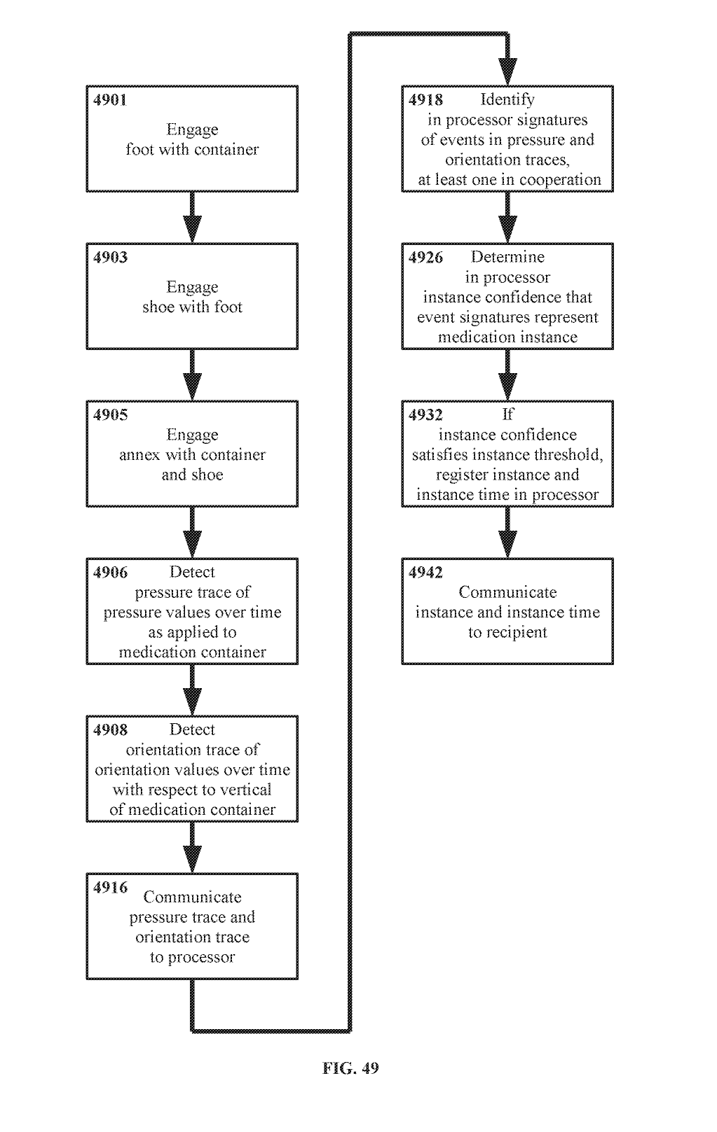

Now with reference to FIG. 49, as has been noted certain arrangements herein may facilitate retrofitting an existing “dumb” container to perform functions as described herein, e.g., determining whether a medication instance has taken place, etc. FIG. 49 shows an example arrangement for such retrofitting. (Structure showing shoes, etc., as may be suitable for such retrofits are shown previously herein, e.g., FIG. 17 through FIG. 31.)

In the example method of FIG. 49, a foot is engaged 4901 with a container that contains a medication. The manner of engagement may vary considerably, e.g., fixedly engaged as by permanent glue or welding, removably engaging as by mechanical latches or temporary adhesive, etc. Alternately, in certain embodiments the foot may simply be the bottom of the container, and/or may be made integrally with the container; in such instance a method for retrofitting may omit step 4901 or an analog thereof.

A shoe is engaged 4903 with the foot. As described in other examples herein, the shoe may include sensors, a processor, etc., and the manner of engagement may vary. Typically, though not necessarily, the shoe may be removably engaged.

In at least certain embodiments, an annex is engaged 4905 with the container, and/or engaged 4905 with the shoe. For example, the annex may take the form of a membrane such as an adhesive label, e.g., with a sensor or probe (such as a pressure sensor or probe) disposed therein. In such case, engaging 4905 the annex with the container then may take the form of wrapping that label around the container as to affix the label thereto. Likewise, engaging 4905 the annex with the shoe may include connecting wires or other links between a sensor or probe as may be present within the annex and a processor, sensor, power source, etc., as may present within the shoe. Not all embodiments necessarily will have or require annexes, thus engaging the annex 4905 to the container may be optional; similarly, not all embodiments that have annexes necessarily will have or require wire connections (or similar) to the shoe, thus engaging 4905 the annex to the shoe also may be optional.

Continuing in FIG. 49, a first data trace is detected 4906, and a second data trace is detected 4908, e.g., by sensors in the shoe that was engaged 4903 with the foot of the container previously. The first and second data traces are communicated 4916 to a processor. In the processor, signatures are identified 4918 within the traces consistent with corresponding events. At least one signature for at least one such event identified 4918 through consideration of the first and second data traces in cooperation. An instance confidence is determined 4926 as to how likely it may be that a medication instance (or other phenomenon) has taken place. The medication instance and medication instance time are registered if that instance confidence is determined 4932 to meet a threshold therefor, and the instance and instance time are communicated 4942 to some recipient.

It should be understood that various features in examples herein may be combined, duplicated, etc. Thus, while the example method of retrofit shown in FIG. 49 references only two data traces, the use of more than two traces as shown for example in FIG. 38 likewise may be suitable for other embodiments.

With reference again to FIG. 45, as noted therein information regarding whether a medication instance has taken place—for example, the time of a medication instance, the interval since the previous medication instance, the dosage of each medication instance, etc.—may serve as to illuminate the degree to which a particular subject is adhering to a prescribed medication regimen. Adherence factors and/or other measures of adherence also may be determined, as previously indicated. However, while the method shown in FIG. 45 provides an example by which such information may be acquired and/or utilized, it is emphasized that other approaches for addressing such data also may be suitable.

For example, many variations of methods as shown herein may also determine adherence similarly. In addition, adherence need not be determined either by an apparatus engaged with a container, nor as part of the operation of such an apparatus. For example, a shoe as shown in certain examples herein may identify signatures, register medication instances, etc., while determinations of adherence may be handled elsewhere, such as by a recipient of the medication instance and medication instance time (and/or other data). As a more concrete example, a shoe fitted to an eyedrop container may collect sensor data, but adherence (whether in the form of adherence factors or otherwise) may be evaluated and/or tracked in some remote processing system to which data is sent. For instance, data could be communicated to a base, to a freestanding device such as a smart phone or personal computer, to the cloud, to a remote database or processing network, etc., with determinations about adherence being made there (in whole or in part).

In addition, regardless of how authenticated adherence data is acquired, so long as that adherence data is indeed authenticated—e.g., being based on sensor data or otherwise “real-world” information as opposed to being subjectively self-reported, etc.—such authenticated adherence data may be further evaluated in a variety of manners.

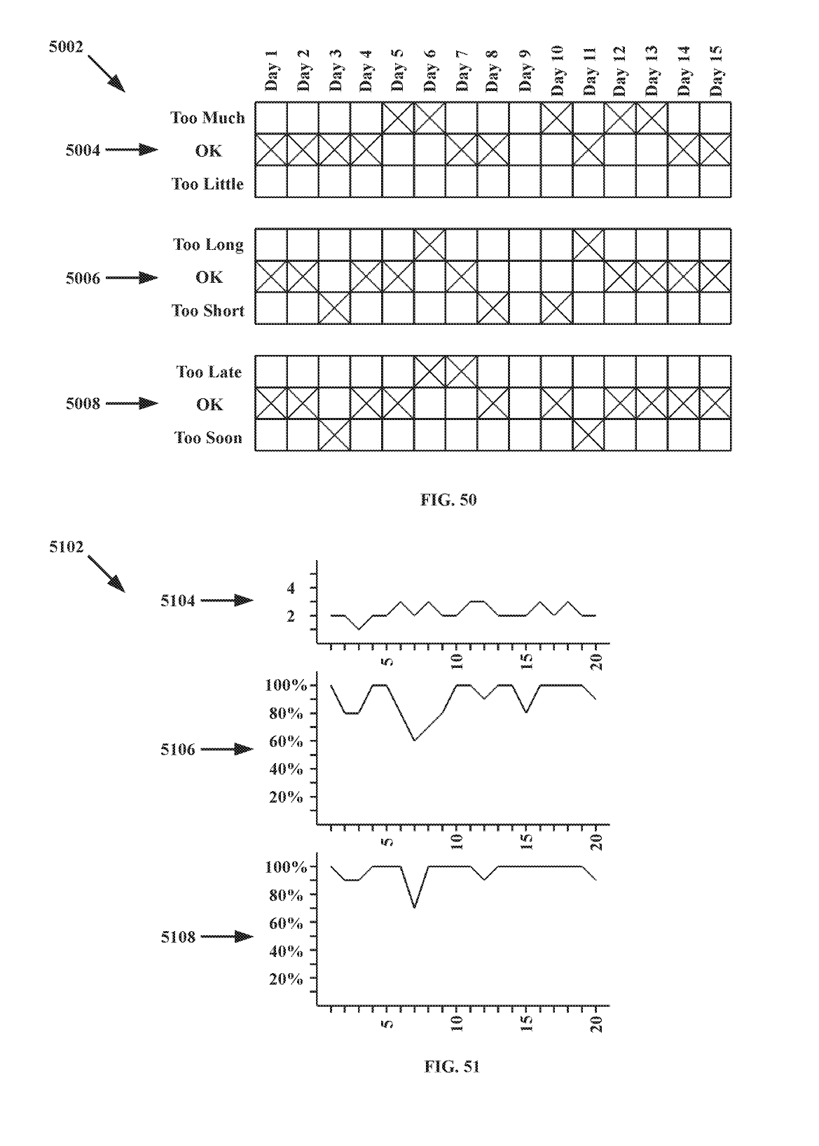

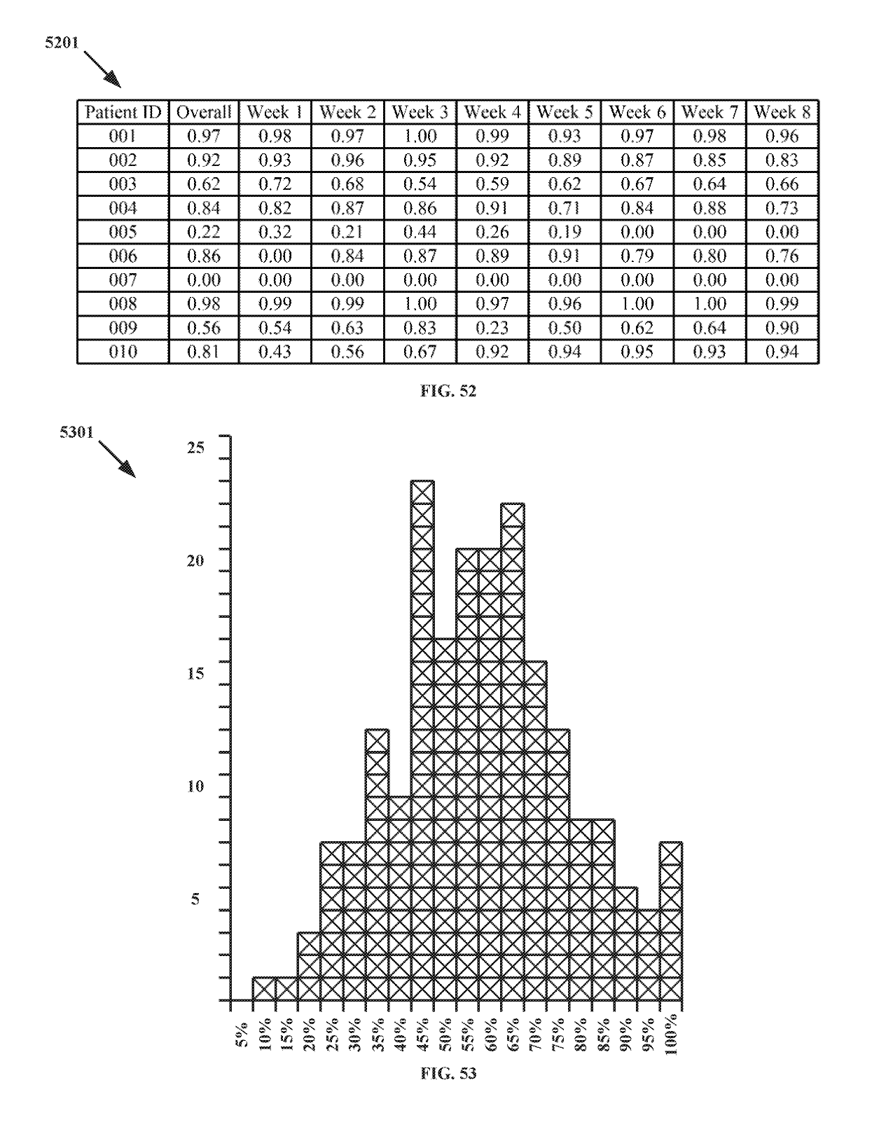

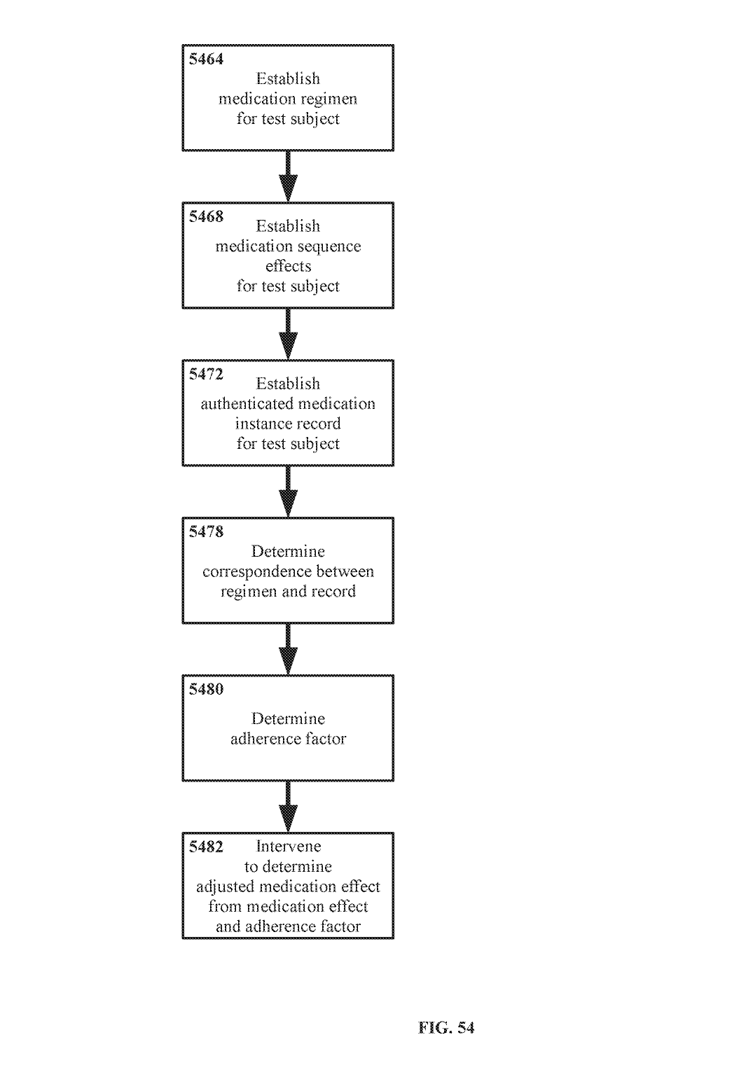

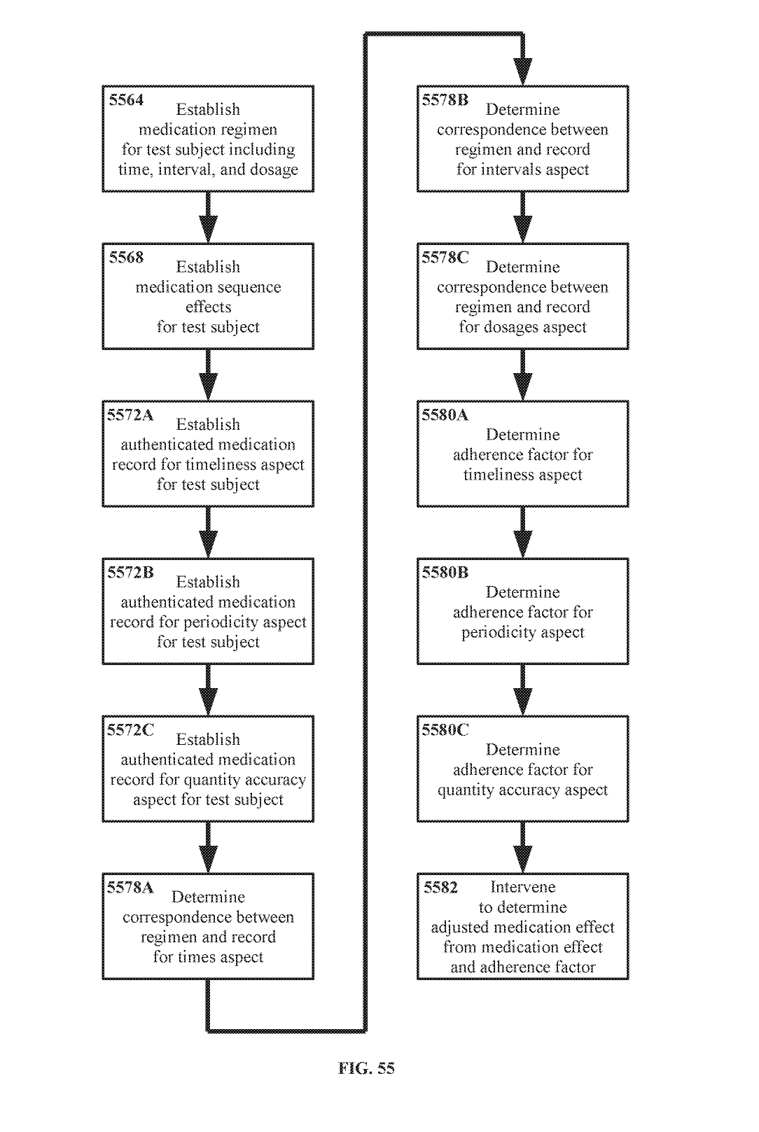

Now with reference collectively to FIG. 50 through FIG. 53, examples are shown of approaches for considering adherence information. These should not be considered limiting, and many other approaches may be suitable. Likewise, although certain examples in FIG. 50 and FIG. 51 address three aspects of adherence—time, interval, and dosage—this too is an example only. Other aspects may be considered, any or all of time, interval, and dosage may be ignored for certain embodiments, and more or fewer total aspects may be considered. In addition, as is addressed in greater detail below with regard to FIG. 52 and FIG. 53, adherence may be considered as a non-specific value, such as a numerical factor without explicitly referencing (or even considering) individual aspects such as time, interval, dosage, etc.

With reference now specifically to FIG. 50, adherence information 5002 is shown for an individual subject taking a medication. In this example, three aspects of adherence information are shown: dosage 5004, interval between doses 5006, and times of doses 5008. The manner by which the adherence information is acquired is not limited, however as described previously medication instances, medication instance times, medication instance dosages, etc. may be obtained through identifying signatures in sensor traces. The medication instance times may be interpreted as (or may be modified to serve as) the times 5008 of doses taken by the subject. If the times 5008 at which doses were taken are known, the intervals 5006 between doses may be determined or at least approximated. And if the dosage 5004 is known, e.g., from a number of pressure spikes corresponding to eyedrops being squeezed from a bottle, then this information too may be considered.

In raw form, medication instance information may not necessarily resemble what is shown in FIG. 50. For example, a medication instance may be represented by a numerical code indicating a specific medication was taken by a specific patient, a medication time may be a numerical representation of time (local standard time, some measure based on processor cycles or other electronic events, etc.), and a dosage may be a number of droplets, a number of pills, the milligrams of medication taken, etc. However, while consideration of such raw data is not prohibited, and indeed adherence information may be considered in any useful form, in practice adherence information may be “distilled” or processed into a form that may be readily understood and acted upon. For example, a printout of times, codes, etc. may not appear somewhat opaque to a patient or even a physician familiar with the system. Thus, as shown in FIG. 50, adherence information 5002 is shown in a somewhat abstracted but potentially useful form.

Namely, as may be seen in FIG. 50, each aspect 5004, 5006, and 5008 is shown in terms of whether the subject matched their prescribed regimen or not on any given day, and if the subject deviated in which direction. The dosage 5004 is marked with Xs indicating that the dose was OK (e.g., per the regimen as prescribed), too much, or too little. For example, if two eyedrops are to be taken, taking two would correspond with “OK”, three or more with “too much”, and one or none with “too little”. While simple, such information may be useful nevertheless. Likewise, the interval 5006 may be marked with an X as “OK” if the medication is taken at intervals specified in the regimen, “too long” if too much time elapsed between doses, and “too short” if doses were taken too close together. Such identifications need not be exact; the “OK” state may for example include some range, such as plus-or-minus 30 minutes, etc. Similarly, the times 5008 may be referred to as “OK” if the medication is taken on time (again, potentially within some range), or “too early” or “too late” if taken too early or late in the day.

In addition, while the arrangement in FIG. 50 shows only three states—essentially, “too high”, “OK”, and “too low”—a similar arrangement could be made with more states, such as a range from 1 to 7 where 4 is ideal, 3 through 1 are progressively too low, and 5 through 7 are increasingly excessive. Likewise, time could be handled differently, e.g., each dose rather than each day, etc. (Though for a once-a-day medication each dose may represent each day.)

Now with reference to FIG. 51, another example of adherence information 5102 for an individual on a medication is shown. Again, aspects of dosage 5104, interval 5106, and time 5108 are shown, though as noted this is an example only.

In the arrangement of FIG. 51, the dosage 5104 is in this instance shown numerically for each of 20 days, e.g., a number of droplets dropped into an eye, tablets taken by mouth, etc. on each day. The range of dosage 5104 as shown varies from 1 to 3 each day, with an average slightly above 2. While a numerical value may reveal the actual dosage (unlike in FIG. 50), observing the plot for dosage 5104 does not necessarily reveal whether the dosage is correct; while it may be assumed that the dose is intended to be 2 given the clustering of dosage 5104 values around 2, in fact the regimen may specify 1, 3, or some other number. Thus, direct numerical data is not necessarily superior in all instances, and will not necessarily be utilized or provided in all embodiments.

Similarly, the interval 5106 plot and time 5108 plot may indicate how far from nominal specified times and intervals a medication was taken each day—for example with a 100% accuracy on time 5106 on day 5 but only 60% accuracy on day 7—but do not necessarily indicate information such as whether a medication was taken too soon or too late, or at intervals too long or too short.

Thus, as shown by comparison of FIG. 50 and FIG. 51, various ways of considering adherence information may have different advantages and disadvantages. Embodiments are not limited with regard to how adherence data may be considered for a given subject and/or medication.

Turning to FIG. 52, therein compiled adherence data 5201 for a number of patients is shown over a period of time. In particular, ten patients are identified (by ID number, and individual compliance factors for each patient for each of 8 weeks is shown, along with an overall average compliance factor. As may be seen, the adherence of patients may vary from week to week, and adherence also may vary from patient to patient; while FIG. 52 does not reflect any particular real-world data set, variation is to be expected

Collective consideration of adherence information 5201 may be useful in various ways. For example, comparing one patient to another may provide insight into why a medication works differently in different patients. Patient 007 shows 0.00 adherence for all 8 weeks; that is, the patient either has not taken the medication at all or (depending on how adherence may be determined in this instance, which may vary at noted) has taken the medication so far deviated from the prescribed regimen as to effectively not take the medication properly at all. Thus, little or no medical effect may be expected for patient 007, and likewise little or nothing in the way of side effects may be expected; if the patient reports substantial medical effects and/or side effects, it may be necessary to search for some other cause than the medication (assuming patient 007's reports are taken at face value).

In addition, other uses for such adherence data 5201 may be found. For example, if one were conducting a clinical trial to determine the effectiveness of a medication, then it may be reasonable to either actively seek to select patients with high overall adherence (such as 001, 002, or 008), and/or to seek to exclude patients with low overall adherence (such as 005 or 007). It may also be worth considering whether patients with good overall adherence but notable low points—such as patient 006 who exhibited 0.86 adherence overall but appears not to have taken the medication at all in the first week (with an adherence of 0.00 for week 1)—may be suitable for certain types of clinical studies.

Furthermore, merely knowing the degree of adherence, even when low, may be useful. When considering test studies, statistical methods may facilitate adjustment of data to compensate for nonadherence. That is, if a medication is not being taken as prescribed, and the manner and degree to which the user is deviating from the prescribed regimen is known, it may be possible to approximate what the full effect of the medication would have been if taken according to the regimen. If authenticated adherence information is available—as opposed to approximations or even poorly-supported guesswork—the statistical analysis may be simplified, and/or the results of statistical analysis may be more accurate, reliable, and/or valid. For purposes of treatment as opposed to research, being aware that a patient is not adhering to a medication regime, and/or the manner and degree of nonadherence, also may facilitate useful intervention. If a patient is not taking their medication, or is not taking their medication as prescribed, it may be possible to improve the patient's adherence in some manner. It may be that patient 007 cannot open the bottle, or has been misinformed of severe (but non-existent) side effects, or cannot afford the medication. In such cases it may be possible to switch to easy-open bottles, counsel the patient regarding their concerns, prescribe a less-expensive medication, or otherwise intervene to improve patient outcomes.

Now with reference to FIG. 53, therein is shown another collective body of adherence information 5301 from numerous patients/test subjects. Unlike FIG. 52, in FIG. 53 the adherence information does not specify individuals, but instead aggregates the adherence of many people to give some indication of how closely a regimen is being followed overall. As may be seen, a large fraction of individuals are between about 45% and 75% adherent (the manner by which adherence is determined is not specified for FIG. 53; as noted such adherence determinations may vary widely and are not limited).

Again, such an approach for considering adherence information may have uses, and/or disadvantages. With no tie to individuals, the arrangement of FIG. 53 may not readily suggest possible interventions for individuals. However, such collected adherence information 5301 may be useful in overall evaluation of the degree to which patients or test subjects are taking their medication as prescribed. (This in turn may suggest collective interventions.) For example, considering collected adherence information 5301 may be useful in laying out studies regarding medication. As a more concrete example, consider a medication wherein it is considered that 75% adherence is the minimum necessary for conclusive results; out of 200 subjects in FIG. 53, 32 exhibit 75% or greater adherence. Assuming the medication to be tested and the pool of potential subjects shows similar adherence distributions (e.g., if the results in FIG. 53 were a pilot study using placebos to determine such an adherence distribution, or were from a similar previous study), then the number of candidates needed to have (for example) 100 subjects at 75% or better adherence may be approximated. (In this instance, about 625 subjects.) The study then may be conducted with valid results, where without such adherence data valid results may not be possible. In addition, if authenticated adherence data for such a study may be determined as the study progresses, those subjects that exhibit insufficient adherence—about 525 of the 625—could be terminated from the study after being identified. Thus, the overall cost and complexity of the study may be decreased once underway. Furthermore, if some manner were found to screen for or otherwise identify high-adherence subjects before beginning a study, it would not be necessary to enroll such subjects in the first place. For example, as suggested above, a pilot study (e.g., with a placebo) could be conducted long enough to reveal adherence levels. Alternately, if some discernible property of individuals with high adherence could be identified, subjects could be recruited based on that property. For example, if (for whatever reason) left-handed Norwegian-American university students were found to be unusually adherent when using inhalers, it may be useful to seek out a population of such students for an inhaler medication study. Such selection may be considered carefully so that the properties under consideration for research subjects do not in some manner bias the data, of course. For example, even if women were found to be more adherent in taking a male-baldness medication than men, deliberately selecting women for a clinical trial of such a medication may not be useful in practice.

As has been described authenticated information regarding how a medication is used may be collected using a range of approaches. Likewise, now with reference collectively to FIG. 54 through FIG. 62, such authenticated information may be further considered for a range of functions, such as selecting patients for clinical trials, selecting medication regimens for patients, etc. These are examples only, and arrangements for utilizing authenticated information are not limited. In particular, while certain approaches presented as examples herein may serve to provide authenticated data, the further consideration of authenticated data is not limited based on how authenticated data is obtained (e.g., whether by examples shown herein or otherwise).

A distinction is again emphasized between authenticated medication information and non-authenticated information such as self-reported information, approximation/estimation, theoretical modeling, etc. Authenticated medication information may be understood as representing empirical, measured, recorded, or (in more colloquial terms) “real” times of medication use, dosages used at those times, and so forth. Such authenticated information may be acquired for example through sensing the manipulation of a medication container using sensors and interpreting/recording that data to identify the medication use, or otherwise collecting data so as to record when/how a medication was used in fact. By contrast, patient self-reporting is notoriously unreliable, while approximation and estimation are by definition imprecise and uncertain. Theoretical modeling is inherently a product of the assumptions made; absent authenticated data on which to base a theoretical model in the first place, the results of theoretical modeling may reflect the expectations of those creating the model and/or the properties of the model itself more than the real-world subject being modeled. Use of authenticated data may produce useful results, where other approaches (even if similar in appearance) may not.

In more colloquial terms, getting an answer and getting a valid answer are two very different things. Getting a valid answer may depend on having reliable data to begin with. Thus, availability and use of authenticated medication use data may be understood as being qualitatively different than otherwise similar approaches utilizing (for example) theoretical modeling to determine how many patients in a clinical study will exhibit which types and levels of nonadherence.

Now with reference specifically to FIG. 54, an example arrangement for adjusting medication data to account for nonadherence during some medication test is presented. A medication regimen for a test subject is established 5464. Typically though not necessarily, the medication may take the form of digital data, such as may be instantiated onto a processor. For example, the name of a medication, a prescribed time of use, a prescribed interval of use, a prescribed dosage, and/or other requirements (e.g., “take with food”, “take before sleeping”, “do not take more frequently than every six hours”, “administer two droplets to each eye”, “do not operate heavy equipment within four hours”, etc.) may be identified, and/or may be instantiated onto a processor. Medication regimens typically may be specified by a physician or other care provider, and may be available therefrom, though this is not limiting.

Medication sequence effects are also established 5468. Medication sequence effects refer to what result the medication produces, and may include intended effects and/or side effects. For example, a medication for treating high blood pressure may have an effect of lowering blood pressure; in such case medication sequence effects may include blood pressure values for the subject. It is noted that medication sequence effects are not necessarily required to be conclusively linked to the medication, nor are medication sequence effects necessarily required to be significant or even non-zero. To continue the example above, the patient taking the blood pressure medication may exhibit lower blood pressure over time, but it may not be required to demonstrate conclusively that the drop in blood pressure is due to the medication. Likewise, it may be that the patient exhibits no change in blood pressure, or even that his or her blood pressure increases. While it may be valid and/or useful to define medication sequence effects as “what the medication did”, it may be equally valid and/or useful to define medication sequence effects as “what happened when the medication was taken”. Similarly, a null result for medication sequence effects (no change in blood pressure, etc.) also may be valid and/or useful.

The “sequence” in “medication sequence effects” may refer to the possibility that a subject may take a medication for some period of time, but that for purposes herein the time of use is not limited. Thus, a single use of a medication once may be considered; using a medication daily for many years likewise may be considered a medication sequence. The number of uses/amount of time considered may considerably vary from one embodiment to another.

Still with reference to FIG. 54, as with the medication regimen typically though not necessarily the medication sequence effects may take the form of digital data, such as may be instantiated onto a processor. For example, blood pressure measurements with times thereof, other numerical or test data, observations by a care provider or medication subject, etc. may be instantiated onto a processor. The contents and form of medication sequence effects are not limited.

An authenticated medication instance record for the test subject is established 5472. A medication instance record refers to one or more medication instances and/or associated data, such as medication instance time, etc. Medication instances, medication instance times, etc. have been previously described herein; the information and form thereof are not limited, though typically but not necessarily an authenticated medication instance record may take the form of digital data, such as may be instantiated onto a processor. In addition, the size of a medication instance record is not limited; the record may address a single use of a medication, or many uses over a period of time.

Continuing in FIG. 54, a correspondence is determined between the medication regimen (as established in 5464) and the medication instance record (as established in 5472). That is, some evaluation is made as to how and/or how closely the way that the patient actually took the medication (the medication instance record) does or does not match the way the patient was intended to take the medication (the medication regimen). The manner of evaluation 5478 is not limited, and may vary at least to some degree based on the form of the medication regimen and medication instance record. Typically, though not necessarily, the correspondence may be determined 5478 mathematically and/or geometrically. Also, typically though not necessarily, the correspondence may be determined 5478 within a processor, for example through executing instructions thereon.

An adherence factor is determined 5480 based on the correspondence between regimen and record as determined 5478. The adherence factor likewise may vary considerably, may vary at least in part based on the form of the relevant information, and is not limited. Typically, the adherence factor 5480 may be a processed value, values, graphical representation, etc. in some form as to be useful for one or more applications. For example, single-aspect numerical adherence factors were shown previously in FIG. 52 and FIG. 53, while FIG. 51 shows a three-aspect graphical representation of adherence and FIG. 50 shows a three-aspect non-numerical adherence factor. In principle an adherence factor may simply be a “data dump” of the entire correspondence determined in step 5478 (and such is not excluded from certain embodiments), but in practice the adherence factor may be configured so as to be more “user-friendly” than a collection of raw data. For example, a single three-place decimal number as shown in FIG. 52 or a percentage value as shown in FIG. 53 may be readily compared, conveniently used in further processing, etc. However, the form and/or contents of the adherence factor is not limited.

Typically, though not necessarily, the adherence factor may be determined 5480 mathematically and/or geometrically. Also, typically though not necessarily, the adherence factor may be determined 5480 within a processor, for example through executing instructions thereon.

Continuing in FIG. 54, with the adherence factor determined an intervention 5482 is made so as to determine adjusted medication sequence effects, based on the medication sequence effects and the adherence factor. For example, if it is known that a particular patient was consistently 92% adherent through a medication trial, it may be possible to statistically adjust the observed effects of the medication during that trial (the medication sequence effects) in order to determine what the medication sequence effects would have been if the patient instead had been consistently 100% adherent. Approaches for determining adjusted medication sequence effects are not limited, and may be determined at least in part by the particulars of the data, medication, etc. in a particular embodiment. Typically, though not necessarily, the adjusted medication sequence effects may be determined 5482 mathematically and/or geometrically. Also typically though not necessarily, the adjusted medication sequence effects may be determined 5482 within a processor, for example through executing instructions thereon.

Determining adjusted medication sequence effects 5482 may be useful in a variety of ways. For example, in a clinical trial to determine the effectiveness of a drug, the effects of that drug may be masked by less-than-perfect adherence on the part of the trial subjects. Adjusting the results based on authenticated medication use data to determine the effectiveness of the drug, as distinct from the question of how well the test subjects followed instructions on taking the drug, may be revealing.

Conversely, it may be useful to determine adjusted medication sequence effects 5482 for lower adherence than observed in practice, rather than for perfect or higher adherence. For example, if it is known or hypothesized that a medication may be taken with 60% to 80% adherence by patients in treatment (as opposed to a controlled study), then a study that exhibits 90% adherence may overstate the probable effects of the drug in normal use by patients. Thus, it may be useful to determine adjusted medication sequence effects 5482 for a 60% to 80% adherence range.

In addition, it may be possible to determine the effect of changes other than adherence rate, if the actual use and effects of a drug are sufficiently well-known (e.g., with authenticated use data). For example, by observing differences in effects and/or side effects among patients with varying degrees of adherence, it may be possible to determine with at least some confidence what effects and side effects a lower dose of the medication may produce. While any such analysis may be limited, the use of authenticated data may provide more reliable results and/or greater flexibility than non-authenticated data such as self-reported data.

Moving on to FIG. 55, as noted it may be suitable to consider medication use and adherence in a variety of forms, such as by considering different aspects of adherence individually. For example, the time of use of a medication, the interval between uses, and the dosage may be addressed separately. FIG. 55 shows such an arrangement.

A medication regimen for a test subject is established 5564. The medication regimen specifies at least the time at which the medication is to be taken, the interval between uses of the medication, and the dosage of medication that is to be taken at each instance of use. Medication sequence effects are also established 5568. It is noted that the medication sequence effects may or may not specifically address time, interval, and accuracy of quantity. A subject could for example be asked whether they felt differently after accidentally taking two doses too close together, tests could be run to measure any such difference, etc. Such specificity in establishing 5568 medication sequence effects is neither prohibited nor required, regardless of whether other steps (e.g., determining adherence factors) does so specialize.

Continuing in FIG. 55, an authenticated medication instance record for the timeliness aspect of adherence (e.g., did the subject take the medication on time?) is established 5572A for the test subject. An authenticated medication instance record for the periodicity aspect of adherence (e.g., did the subject take the medication at the right intervals?) is also established 5572B for the test subject, and an authenticated medication instance record for the quantity accuracy aspect of adherence (e.g., did the subject take the right amount of medication?) is established 5572C for the test subject. Steps 5572A, 5572B, and 5572C may be considered together as a single step, even when evaluated separately; thus certain embodiments fitting the arrangement in FIG. 54 also could fit the arrangement in FIG. 55 (e.g., in that step 5472 in FIG. 54 could be seen as encompassing steps 5572A, 5572B, and 5572C in FIG. 55.). As noted with regard to establishing an authenticated medication instance record overall in FIG. 54, establishing aspects thereof is not limited with regard to form, manner, contents, etc.

A correspondence is determined 5578A between the time aspect of the medication regimen and the medication instance record; a correspondence is determined 5578B between the interval aspect of the medication regimen and the medication instance record; and a correspondence is determined 5578C between the accuracy aspect of the medication regimen and the medication instance record. Again, it may be equally suitable to view steps 5578A, 5578B, and 5578C as parts of a single step.

An adherence factor for timeliness is determined 5480A based on the correspondence between regimen and record as determined 5478A; an adherence factor for periodicity is determined 5480B based on the correspondence between regimen and record as determined 5478B; and an adherence factor for accuracy is determined 5480C based on the correspondence between regimen and record as determined 5478C. It may be equally suitable to view steps 5580A, 5580B, and 5580C as parts of a single step.

Regardless of the manner in which adherence is determined 5580A, 5580B, and 5580C, and/or the form/contents of the adherence factor, an intervention 5582 is made to determine adjusted medication sequence effects. The adjusted medication effects may be determined 5582 individually, e.g., for time, interval, and dosage aspects, but even so such determinations may be considered as a single determination (as shown in FIG. 55). References to such aspects as distinct or unified is not limited, and in practice whether aspects are treated as distinct or unified may not be discernible or may not even have any functional difference for at least certain embodiments

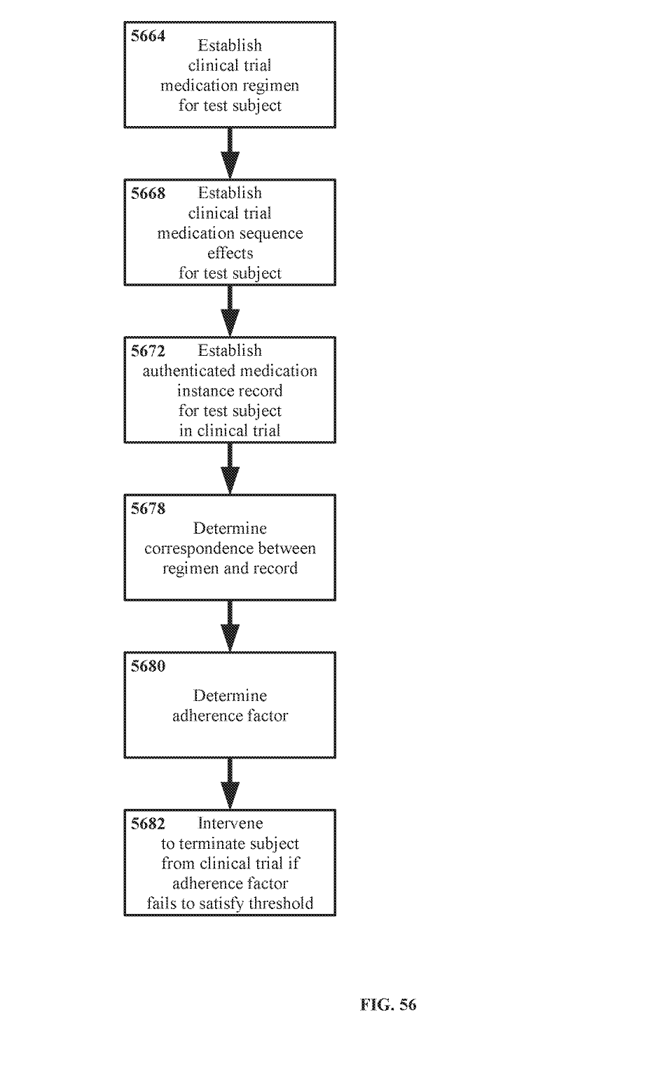

Moving on to FIG. 56, it may be useful in certain instances to remove test subjects from a test if those subjects exhibit poor adherence. A medication regimen for a test subject is established 5664 in a clinical trial. (Reference to a clinical trial in FIG. 56 is an example only; other examples refer to trials nonspecifically, while yet other examples herein refer to patient treatment. These examples are presented for clarity, and are not limiting.)

Medication sequence effects are established 5668. It is noted that the medication sequence may not represent a full trial, nor is such required; as described previously a “sequence” is not limited with regard to time, dosages, etc. Indeed, in the example of FIG. 56, the medication sequence may be presumed to be incomplete (once a trial is complete, terminating participation of a subject may be a moot point).

An authenticated medication instance record for the test subject is established 5672. A correspondence is determined 5678 between the medication regimen and the medication instance record. An adherence factor is determined 5680 based on the correspondence between regimen and record.

With the adherence factor determined an intervention 5682 is made, terminating the subject from the trial if the subject's adherence factor does not satisfy some threshold therefor. The form and nature of an adherence factor threshold may vary considerably, and is not limited. For example, for a numerical, single-value adherence factor the threshold may be “at least 0.50 adherence”, etc. Typically, though not necessarily, the adherence threshold may be mathematical and/or geometric, and determination of whether the threshold is satisfied may be determined mathematically and/or geometrically. As noted with regard to confidence thresholds previously herein, an adherence threshold is not necessarily a simple “cut-off line”; though such cut-offs are not prohibited, other and/or more complex evaluations (e.g., considering multiple aspects of adherence, weighting one aspect over others, etc.) may be suitable. Also typically though not necessarily, the determination of whether the threshold is satisfied may be made within a processor, for example through executing instructions thereon.

Removing nonadherent subjects from a clinical trial as in FIG. 56 may produce various advantages. For example, the number of subjects in a clinical trial typically may impact the cost, the complexity, the number of people needed to staff the trial, etc. Such impacts may be non-linear; that is, doubling the subject pool for a trial may more than double the cost. If a particular subject is exhibiting such low adherence as to not contribute enough to the trial to justify the costs thereof, it may be useful to terminate that subject's part in the trial. In addition, low adherence may suggest other concerns (whether for the trial itself, the subjects, etc.), and determining adherence at some point during a trial, or on a periodic, ongoing, or even real-time basis throughout a trial, may be revealing. Thus, a range of other interventions may be suitable rather than merely terminating the subject. For example, the subject may be counseled, misunderstandings corrected, problematic aspects of the trial modified, etc.

Such variability in intervention likewise is true for other examples herein, including but not limited to FIG. 54 through FIG. 62; while examples of intervention may be shown therein, intervention is not limited only to those examples shown.

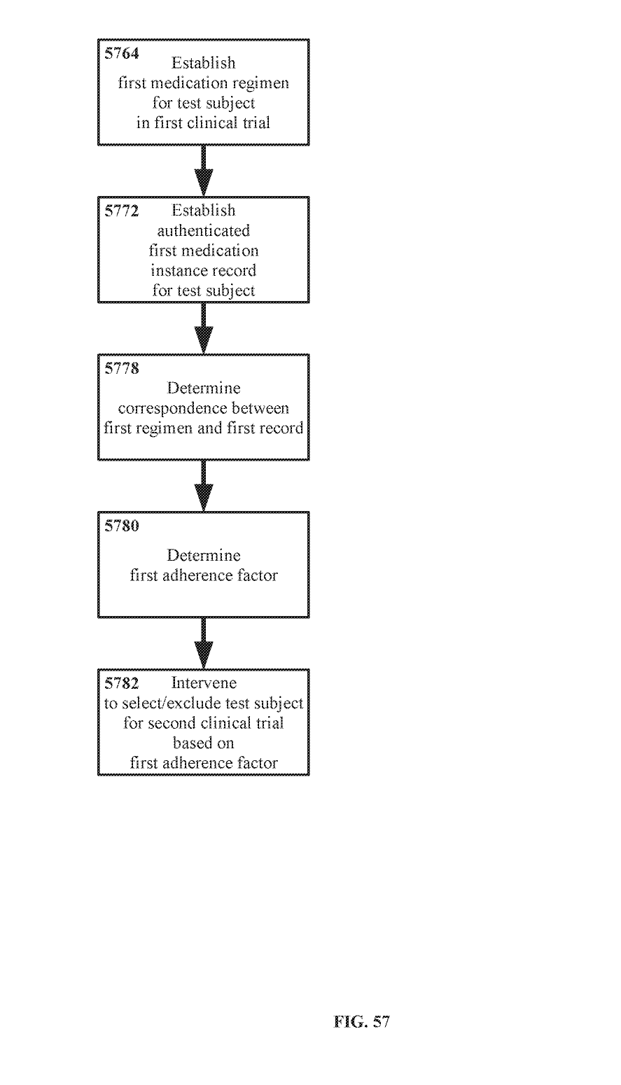

Now with reference to FIG. 57, interventions based on authenticated medication data and/or adherence determinations also may include selecting subjects before the trial is underway. While it may be arguable as to whether such a preemptive action literally constitutes “intervention”, the choice of name for such action does not alter the usefulness of such an approach. In addition, subject selection may take place after design and/or development of a study has begun, thus at least arguably choosing selection criteria may be considered an intervention in a project already underway.

In FIG. 57, a medication regimen for a test subject is established 5764 in a first clinical trial. As noted with regard to FIG. 56, a clinical trial is an example only; an approach similar to FIG. 57 but relying on adherence to previous treatment, rather than a trial, may be suitable.

In addition, it is noted that neither trial nor treatment necessarily assumes a significant medical effect took place, or even was intended. For example, the first clinical trial referenced in 5764 may be a form of “pre-trial”, such as several weeks of trial subjects taking placebos to generate initial adherence data for the subjects. For the purposes of FIG. 57, the actual effects of the medication may be unimportant; medication sequence effects are not indicated as being established in FIG. 57, not being considered as part of the example method. (Though establishing medication sequence effects is not prohibited.)

Regardless, an authenticated medication instance record for the test subject on the first medication is established 5772. A correspondence is determined 5778 between the first medication regimen and the first medication instance record. A first adherence factor is determined 5780 based on the correspondence between first regimen and first record. Thus, by step 5780 in FIG. 57, some measure of the degree to which the subject has taken a first drug according to specified instructions has been determined.

With that first adherence factor determined, an intervention 5782 is made. In the intervention 5782, the test subject is selected for participation in as a subject in a second clinical trial, or excluded from participation as a subject in that second trial, based at least in part on that subject's first adherence factor from the first trial. Thus, the subject's previous record of adherence may be considered as a selection criterion when choosing test subjects for a new trial. Past performance of a person taking a medication may provide some indication of future performance in the same regard.

Such adherence information usefully may be retained over time, whether derived from testing or from actual medical treatment. Given sufficient data on a sufficient number of persons, it may be possible to establish a “rating system” of sorts, where test subjects may be selected from a well-defined pool of persons with a documented history of suitable adherence for whatever new research may be under consideration. As a more concrete example, potential subjects could be rated in on a “1 to 5 star” range with regard to overall adherence, with regard to adherence for particular types of medication (eyedrops, inhalers, pills, etc., if some difference exists), with regard to aspects such as accurate dosage, and so forth. Such a system may still retain anonymity so as to maintain double-blind conditions (and respect subject privacy). Researchers seeking test subjects may have limited access to information beyond adherence so as to avoid potential bias; however other information, such as relevant medical conditions, etc. may be made available.

With regard to additional information, in certain instances it may be useful to include geographical information, such that a researcher may locate local test subjects. However, given such a system, and given suitable arrangements for acquiring the relevant medication data, limiting research to local subjects may not be necessary. If researchers can search based on adherence, and acquire authenticated medication instance records during research, it may be suitable to conduct research with little or even no face-to-face contact between researchers and participants. Such arrangements may expand the potential pool of test subjects considerably; rather than limiting a clinical trial to (for example) individuals within close proximity of a research lab, the trial may be performed across an entire nation. Such flexibility may also contribute to the validity of at least certain types of research; the populations near major research centers do not necessarily represent regional, national, or global populations in terms of demographics (e.g., age, ethnicity, gender, etc.) or social factors (income level, immediate family size, profession, etc.), factors which potentially may impact some forms of research.

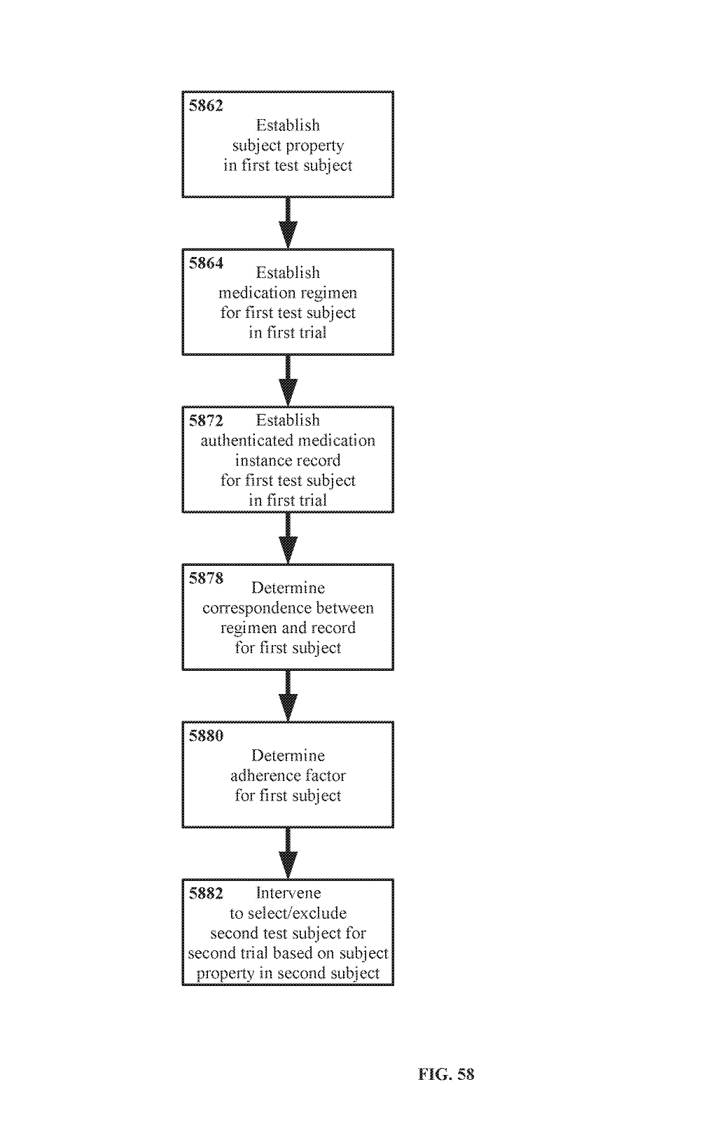

With reference now to FIG. 58, while FIG. 57 provided an example of selecting subjects with consideration given to past adherence, it also may be suitable to select subjects with consideration given to similarities to other subjects. That is, certain properties may correlate with certain degrees of adherence. If those properties may be identified, then it may be possible in turn use those properties to identify subjects with a desired adherence for future research.

In FIG. 58, a subject property is established 5862 for a first test subject. The subject property is not limited, except in that the subject property must correlate or at least have some reasonable expectation of correlating with the subject's adherence. That is, any property may be suitable, so long as that property and some desired degree or form of medication adherence (not necessarily high adherence) are in some manner linked. It is not necessary to identify an underlying reason (if any) for the correlation; if volleyball fans can be identified as more likely to show high adherence with taking eyedrops as prescribed, that correlation in itself may be sufficient regardless of why (or whether an explanation is even known). Likewise, if persons answering questions on a questionnaire in certain fashion are found to show some desirable degree or form of adherence, explaining why may not be necessary (even if the questions appear irrelevant or totally nonsensical). The correlation itself is of interest: so long as desired adherence trait B goes along with subject property A, why or how is not limiting. (Certain links that may pose risks of bias, health risk, or other problems may of course be considered, and such other factors as may be considered are themselves not limited.)

In addition, a property need not correlate with a useful feature in order to be considered. For example, discovering that (for whatever reason) people who leave the water running while they brush their teeth exhibit unusually low adherence may be a useful correlated property. Prospective subjects could be questioned and filtered out of a trial on that basis (and/or flagged in a database of subjects such as described with regard to FIG. 57).

Still with reference to FIG. 58, with a suitable subject property established 5862 for a first test subject, a medication regimen for the first test subject is established 5864. An authenticated medication instance record for the first test subject on the first medication is established 5872. A correspondence is determined 5878 between the first medication regimen and the first medication instance record. An adherence factor for the first subject is determined 5880 based on the correspondence between first regimen and first record. With adherence factor determined for the first subject, an intervention 5882 is made. In the intervention 5882, the second test subject is selected for participation as a subject in a second clinical trial, or excluded from participation as a subject in that second trial, based at least in part on whether the second subject exhibits the same or a similar subject property as the first subject in the first trial. Thus, the adherence of other subjects who in some manner are similar and/or behave similarly to subject candidates for a new trial may be considered as a selection criterion when choosing subjects for that new trial. Similarities between new subjects and successful past subjects may provide some indication of the future performance of the new subjects.

As noted with regard to FIG. 57, such “similar property” adherence information also may be retained over time, and may be considered in a rating system, etc., as previously described. Characterizing likely adherence of a person (or population) based on similarity or other correlation to other persons (or populations) may enable access to a broad range of possible test subjects, such as those who have no personal history of authenticated adherence information. Such property-based identification of likely subjects also may simplify the process of finding subjects; if (for example) stamp collecting enthusiasts are found to have desirable adherence, then it may be possible to seek out research subjects through existing channels for stamp enthusiasts (web sites, magazines, word-of-mouth, etc.).

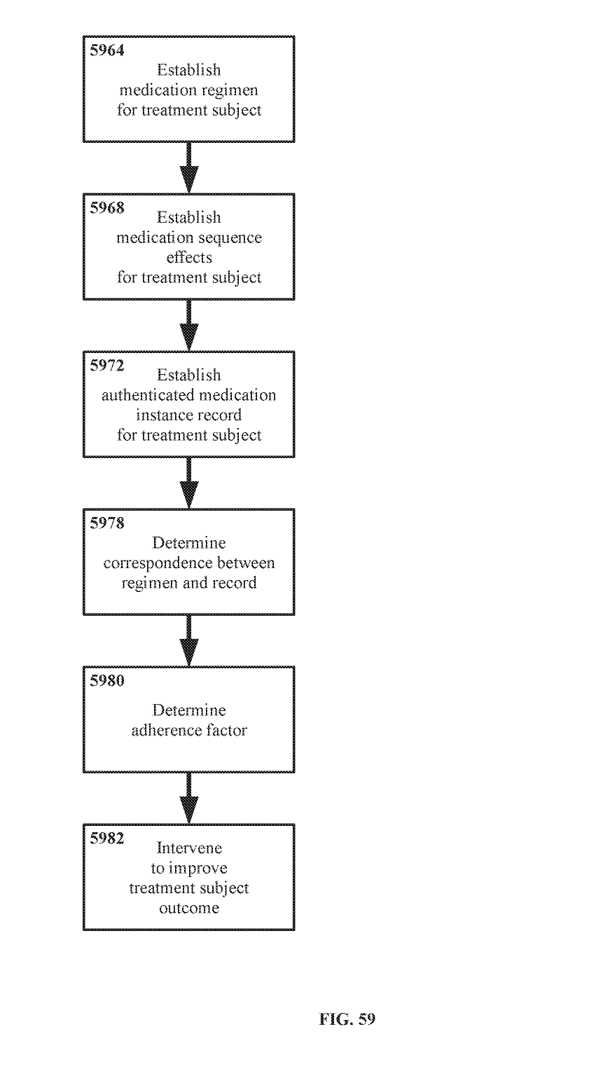

Now with reference to FIG. 59, as noted authenticated adherence information may be usefully considered with to patient treatment as well as research. In FIG. 59, a medication regimen for a medical treatment subject is established 5964. A medication effects sequence for the subject undergoing treatment is established 5968. An authenticated medication instance record for the treatment subject is also established 5972. A correspondence is determined 5978 between the regimen and the record, and an adherence factor is determined 5980. An intervention is made 5982 to improve the outcome of the subject's treatment.

The nature of the intervention 5982 to improve patient outcome is not limited. Where for certain previous examples addressing research intervention may have been directed to (though not limited to) improving research accuracy, decreasing research cost or duration, etc., in FIG. 59 intervention aims to (in colloquial terms) “make the patient better”. For example, if adherence is low a care provider may judge that the patient could benefit from higher adherence. The care provider may counsel the patient, discussing the reasons why the patient shows low adherence and what might be useful in improving adherence. However, it also may be suitable for a care provider to alter the regimen in some manner. For example, rather than try to improve adherence, the care provider may prescribe a different medication that is less sensitive to low adherence; even though the nominal effect of the original medication may be greater in principle, the actual effect of the new medication as taken by the patient may be superior in practice. As another example, if the patient is having difficulty swallowing large pills, it may be possible to provide the same medication in physically smaller units, e.g., four one-hundred milligram tablets instead of one four-hundred milligram tablet.

Other arrangements also may be suitable. For example, if the patient is utilizing a “smart” container as described elsewhere herein, the container may also have the capability (or be modified to have the capability) to provide reminders to the patient. An LED tell-tale normally indicating power status may be made to flash on and off if a patient has missed their dose, for example, or a display screen may be incorporated into a container (or a shoe, a base, etc.) with reminders and/or other information.

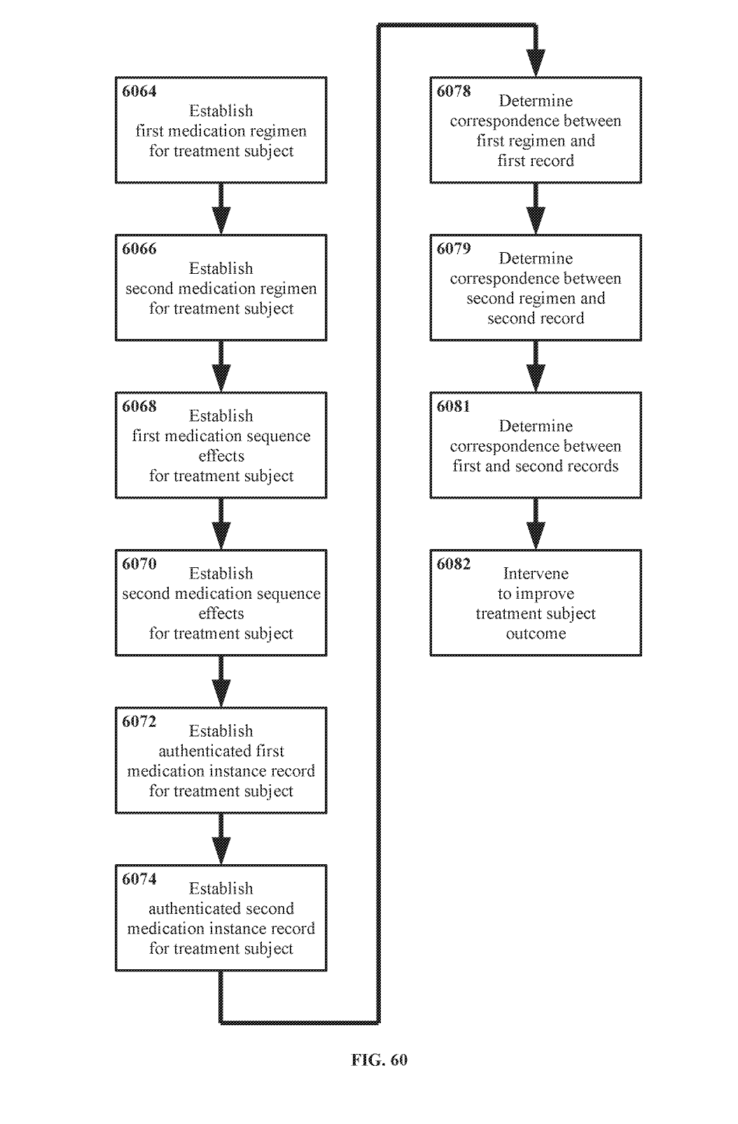

Now with reference to FIG. 60, another example of intervention to improve patient treatment outcome is shown. Interactions between medications may have negative effects (in some cases quite severe), and those interactions may be more or less pronounced depending on how and when the medications are taken. Expected interactions when medications are prescribed may rely on use of medications exactly per respective regimens, but in practice patients may deviate from regimens. However, if authenticated medication instance information is available, it may be useful to identify high and/or low risk arrangements of medications as those medications are actually taken and intervene to improve patient outcome.

Now with reference to FIG. 60, a first medication regimen for a subject is established 6064. A second medication regimen, i.e., for a second medication taken by the same subject, is also established 6066. A first medication effects sequence for the subject is established 6068, and a second medication effects sequence for the subject also is established 6070. An authenticated first medication instance record for the subject is established 6072, and a second medication instance record for the subject is established 6074. A first correspondence is determined 6078 between the first regimen and the first record, and a second correspondence is determined 6079 between the second regimen and the second record. Similar steps have been described previously herein.

Continuing in FIG. 60, a correspondence is determined 6081 between the first and second records. That is, the manner in which the patient takes the first medication (per authenticated data, rather than assuming 100% adherence to the regimen) is compared with the manner in which the patient takes the second medication (likewise per authenticated data). Such correlations may be revealing; for certain medications blood levels may spike initially, decline relatively quickly to a plateau, and then continue to decline more gradually. Two such profiles from two different medications may offer a number of possible coinciding events: two spikes together, a spike in one with a low level of the other, etc. Depending on the medications in question, such coinciding events may be undesirable. For instance, it may be that two such medications are generally compatible in most conditions, but produce unpleasant and/or dangerous side effects if both have high blood levels at the same time (if the spikes coincide).

In addition to comparing first and second records 6081, and/or as part of such a step, it also may be useful (though not required) to compare first and second medication sequence effects. For example, the patient may report side effects without knowing what has caused those side effects (coinciding blood level spikes). It is noted that not all medications will produce spikes of the same duration or at the same amount of time after being taken, thus taking two medications at different times may not assure that blood level spikes in those medications do not coincide.

Still with reference to FIG. 60, an intervention is made 6082 to improve the outcome of the subject's treatment. As noted with regard to FIG. 59, interventions may include but are not limited to counseling the patient (or some person assisting with the patient's care) and/or altering one or both medication regimens. For example, a patient may be reminded or advised as to how long after administration each medication produces peak blood levels, and encouraged to adhere more strictly to the regimens for one or both medications to avoid coinciding spikes. Alternately, one or both medications may be changed to avoid side effects; again, even if the two medications are compatible in principle if taken according to the regimen, if in practice the patient is taking those medications in a manner that produces coinciding spikes (or other undesirable coincidences) it may be more useful to switch to medications that are less effective in theory but safer in practice for this particular patient.

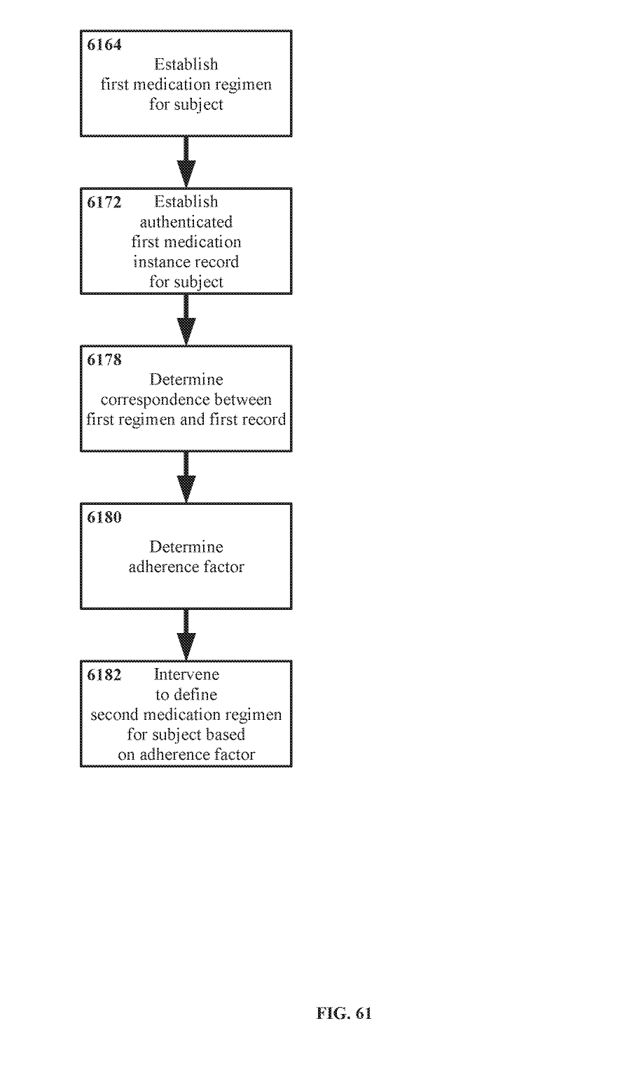

Now with reference to FIG. 61, as noted “intervention” as used herein does not necessarily imply action after research and/or treatment has begun. Rather, intervention may apply to the process of a care provider prescribing a medication in the first place, which also may be useful.

In FIG. 61, a medication regimen for a subject is established 6164 (whether in a clinical trial, treatment, etc.). An authenticated medication instance record for the test subject on the first medication is established 6172. A correspondence is determined 6178 between the first medication regimen and the first medication instance record. An adherence factor is determined 6180 based on the correspondence between first regimen and first record. With the adherence factor determined, an intervention 6182 is made. A second medication regimen, e.g., for a second medication, is defined 6182 based at least in part on the adherence factor. Thus, the subject's record of adherence may be considered not only in changing the subject's current medication but in selecting future medications and how those medications are to be taken. Typically though not necessarily, such definition of a medication regimen may be carried out by a physician or other care provider.

As noted already with regard to FIG. 57, subject adherence information usefully may be retained over time, whether derived from testing or from actual medical treatment, for use in either future testing or future medical treatment (or some other relevant application). Patients as well as test subjects may be “rated”, with that rating information then made available to care providers; thus even a physician who has no personal experience with a given patient may nevertheless rely on authenticated medication information in judging what new medication(s) may be useful to that patient, and in what manner those medications may be usefully taken by that patient.

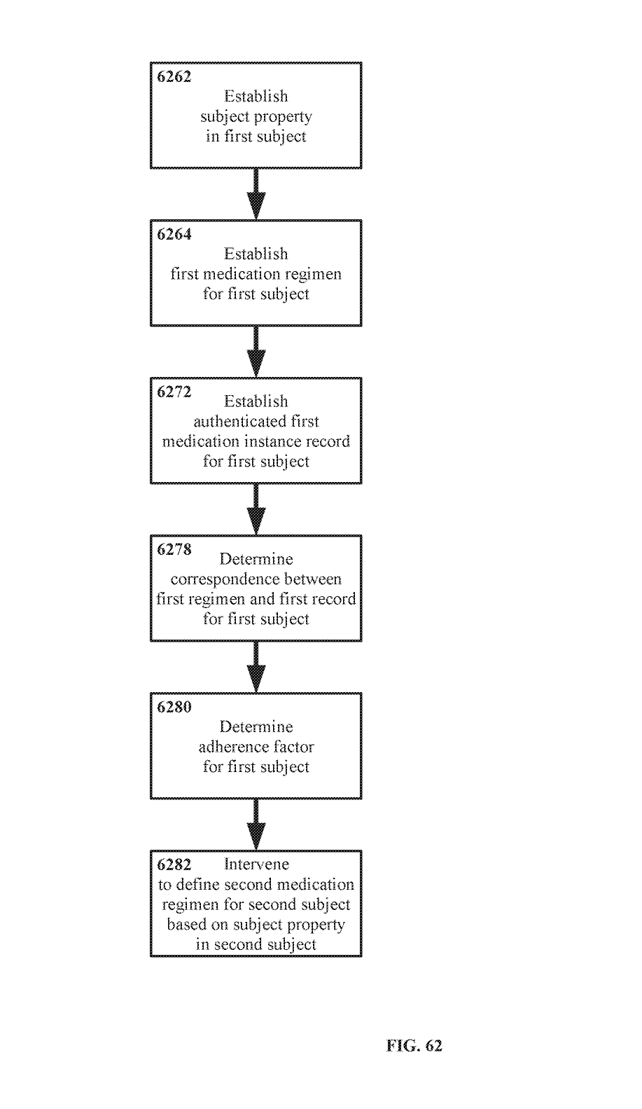

Turning to FIG. 62, in the example therein a subject property is established 6262 for a first test subject. A first medication regimen for the first test subject is established 6264. An authenticated first medication instance record for the first test subject on the first medication is established 6272. A correspondence is determined 6278 between the first medication regimen and the first medication instance record. An adherence factor for the first subject is determined 6280 based on the correspondence between first regimen and first record. With the adherence factor determined for the first subject, an intervention 6282 is made. A second medication regimen, e.g., for a second medication (though the medication may be the same; it is not required for the first and second medications to be different), is defined 6282 based at least in part on whether the second subject exhibits the same or a similar subject property as the first subject. Thus, similarities between subjects with authenticated adherence information and subjects without may provide some indication of the future adherence of the new subjects. (Indeed, it may be useful in certain embodiments to determine a preliminary or provisional adherence for a subject, approximating the subject's anticipated adherence based on whatever properties that subject exhibits that correlate with adherence.) Again typically though not necessarily, such definition of a medication regimen may be carried out by a physician or other care provider.

As already noted with regard to FIG. 61, subject adherence information usefully may be retained over time. With a sufficiently large database, and a sufficiently large number of correlating properties, it may be possible to reliably approximate an expected adherence profile for subjects based on their properties, even if no authenticated adherence data is initially available for a given subject. Such a database also may prove illuminating to research in itself, with sufficient numbers of subjects, quantities of authenticated data, correlated properties, etc.

In addition, with regard to FIG. 54 through FIG. 62 collectively, while examples therein addressed medication, embodiments are not limited only to those related to the use of medication. Other medical treatments such as therapeutic exercises may be suitable, so long as suitable authenticated information is available to address matters of adherence. Likewise, non-medical activities also may be suitable, and the types of activities that may be evaluated with regard to adherence are not limited.

Processing System

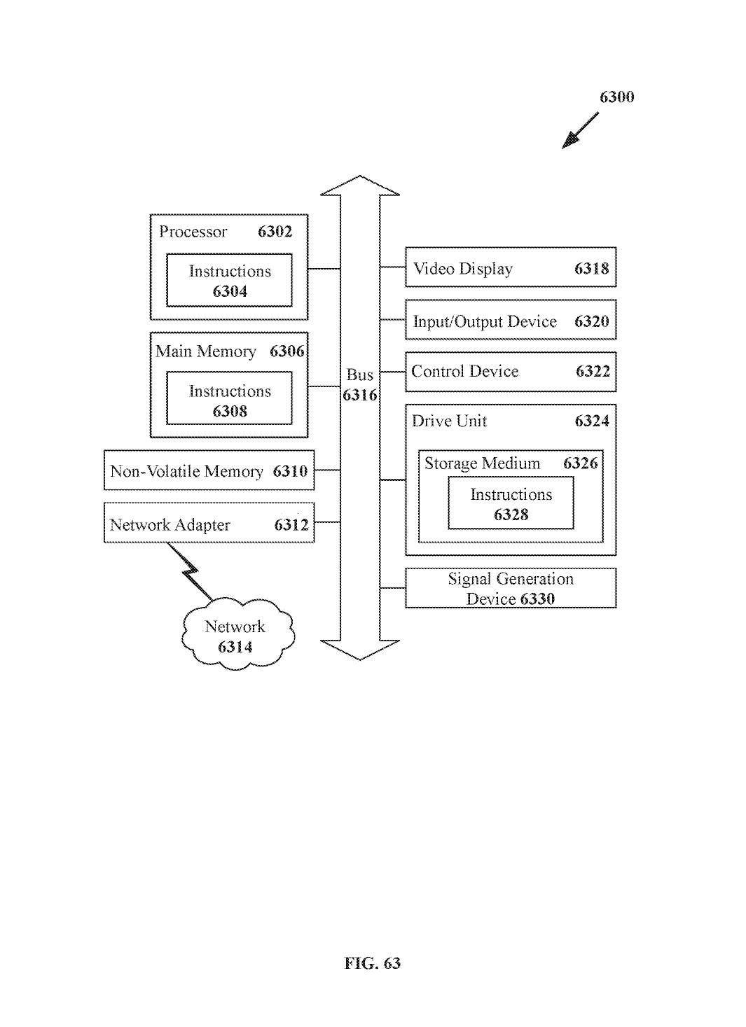

FIG. 63 is a block diagram illustrating an example of a processing system 6300 in which at least some operations described herein can be implemented. The processing system may include one or more central processing units (“processors”) 6302, main memory 6306, non-volatile memory 6310, network adapter 6312 (e.g., network interfaces), video display 6318, input/output devices 6320, control device 6322 (e.g., keyboard and pointing devices), drive unit 6324 including a storage medium 6326, and signal generation device 6330 that are communicatively connected to a bus 6316. The bus 6316 is illustrated as an abstraction that represents any one or more separate physical buses, point to point connections, or both connected by appropriate bridges, adapters, or controllers. The bus 6316, therefore, can include, for example, a system bus, a Peripheral Component Interconnect (PCI) bus or PCI-Express bus, a HyperTransport or industry standard architecture (ISA) bus, a small computer system interface (SCSI) bus, a universal serial bus (USB), IIC (I2C) bus, or an Institute of Electrical and Electronics Engineers (IEEE) standard 1394 bus, also called “Firewire.”

In various embodiments, the processing system 6300 operates as a standalone device, although the processing system 6300 may be connected (e.g., wired or wirelessly) to other machines. For example, in some embodiments components of the processing system 6300 are housed within a computer device used by a user to access an interface having skin care products or skin care regimens, while in other embodiments components of the processing system 6300 are housed within a network-connected container that holds one or more skin care products. In a networked deployment, the processing system 6300 may operate in the capacity of a server or a client machine in a client-server network environment, or as a peer machine in a peer-to-peer (or distributed) network environment.

The processing system 6300 may be a server, a personal computer (PC), a tablet computer, a laptop computer, a personal digital assistant (PDA), a mobile phone, a processor, a telephone, a web appliance, a network router, switch or bridge, a console, a hand-held console, a (hand-held) gaming device, a music player, any portable, mobile, hand-held device, or any machine capable of executing a set of instructions (sequential or otherwise) that specify actions to be taken by the processing system.

While the main memory 6306, non-volatile memory 6310, and storage medium 6326 (also called a “machine-readable medium) are shown to be a single medium, the term “machine-readable medium” and “storage medium” should be taken to include a single medium or multiple media (e.g., a centralized or distributed database, and/or associated caches and servers) that store one or more sets of instructions 6328. The term “machine-readable medium” and “storage medium” shall also be taken to include any medium that is capable of storing, encoding, or carrying a set of instructions for execution by the processing system and that cause the processing system to perform any one or more of the methodologies of the presently disclosed embodiments.

In general, the routines executed to implement the embodiments of the disclosure, may be implemented as part of an operating system or a specific application, component, program, object, module or sequence of instructions referred to as “computer programs.” The computer programs typically comprise one or more instructions (e.g., instructions 6304, 6308, 6328) set at various times in various memory and storage devices in a computer, and that, when read and executed by one or more processing units or processors 6302, cause the processing system 6300 to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Further examples of machine-readable storage media, machine-readable media, or computer-readable (storage) media include, but are not limited to, recordable type media such as volatile and non-volatile memory devices 6310, floppy and other removable disks, hard disk drives, optical disks (e.g., Compact Disk Read-Only Memory (CD ROMS), Digital Versatile Disks, (DVDs)), and transmission type media such as digital and analog communication links.

The network adapter 6312 enables the processing system 6300 to mediate data in a network 6314 with an entity that is external to the computing device 6300, through any known and/or convenient communications protocol supported by the processing system 6300 and the external entity. The network adapter 6312 can include one or more of a network adaptor card, a wireless network interface card, a router, an access point, a wireless router, a switch, a multilayer switch, a protocol converter, a gateway, a bridge, bridge router, a hub, a digital media receiver, and/or a repeater.

The network adapter 6312 can include a firewall that can, in some embodiments, govern and/or manage permission to access/proxy data in a computer network, and track varying levels of trust between different machines and/or applications. The firewall can be any number of modules having any combination of hardware and/or software components able to enforce a predetermined set of access rights between a particular set of machines and applications, machines and machines, and/or applications and applications, for example, to regulate the flow of traffic and resource sharing between these varying entities. The firewall may additionally manage and/or have access to an access control list which details permissions including for example, the access and operation rights of an object by an individual, a machine, and/or an application, and the circumstances under which the permission rights stand.

As indicated above, the computer-implemented systems introduced here can be implemented by hardware (e.g., programmable circuitry such as microprocessors), software, firmware, or a combination of such forms. For example, some computer-implemented systems may be embodied entirely in special-purpose hardwired (i.e., non-programmable) circuitry. Special-purpose circuitry can be in the form of, for example, application-specific integrated circuits (ASICs), programmable logic devices (PLDs), field-programmable gate arrays (FPGAs), etc.

Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling others skilled in the relevant art to understand the claimed subject matter, the various embodiments, and the various modifications that are suited to the particular uses contemplated.

While embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Although the above Detailed Description describes certain embodiments and the best mode contemplated, no matter how detailed the above appears in text, the embodiments can be practiced in many ways. Details of the systems and methods may vary considerably in their implementation details, while still being encompassed by the specification. As noted above, particular terminology used when describing certain features or aspects of various embodiments should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless those terms are explicitly defined herein. Accordingly, the actual scope of the invention encompasses not only the disclosed embodiments, but also all equivalent ways of practicing or implementing the embodiments under the claims.

The language used in the specification has been principally selected for readability and instructional purposes, and it may not have been selected to delineate or circumscribe the inventive subject matter. It is therefore intended that the scope of the invention be limited not by this Detailed Description, but rather by any claims that issue on an application based hereon. Accordingly, the disclosure of various embodiments is intended to be illustrative, but not limiting, of the scope of the embodiments, which is set forth in the following claims.

What is claimed is:

1. A method comprising:
with a pressure sensor engaged with a medication container, detecting a pressure trace comprising a plurality of pressure values over time corresponding with an application of pressure to said medication container over time;
with a disposition sensor engaged with said medication container, detecting a disposition trace comprising a plurality of disposition values over time corresponding with a disposition of said medication container over time;
communicating said pressure and disposition traces to a processor;
in said processor, identifying a plurality of signatures in said pressure and disposition traces, identifying at least one of said signatures in said pressure and disposition traces in cooperation, each said signature being consistent with a corresponding event associated with a medication instance, said medication instance comprising dispensing a medication from said medication container;
in said processor, determining a medication instance confidence that said plurality of signatures collectively represents said medication instance based on said signatures;
in response to said medication instance confidence satisfying a threshold therefor, registering said medication instance and a medication instance time with said processor; and
communicating said medication instance and medication instance time to a recipient.

2. The method of claim 1, comprising:
with an outlet obstruction sensor engaged with said medication container, detecting an outlet obstruction trace comprising a plurality of outlet obstruction values over time corresponding with whether an outlet of said medication container is obstructed over time;
communicating said outlet obstruction trace to said processor; and
in said processor, identifying said signatures from said pressure, disposition, and outlet obstruction traces, identifying at least one of said signatures with said pressure, disposition, and outlet obstruction traces in cooperation.

3. The method of claim 1, comprising:
with a quantity sensor engaged with said medication container, detecting a quantity trace comprising a plurality of quantity values over time corresponding with a quantity of said medication in said medication container over time;
communicating said quantity trace to said processor; and
in said processor, identifying said signatures from said pressure, disposition, and quantity traces, identifying at least one of said signatures with said pressure, disposition, and quantity traces in cooperation.

4. The method of claim 1, comprising:
with an emergence sensor engaged with said medication container, detecting an emergence trace comprising a plurality of emergence values over time corresponding with an emergence of said medication from said medication container over time;
communicating said emergence trace to said processor; and
in said processor, identifying said signatures from said pressure, disposition, and quantity traces, identifying at least one of said signatures with said pressure, disposition, and emergence traces in cooperation.

5. The method of claim 1, comprising:
in said processor, determining a plurality of event confidences that each said signature represents said corresponding event from said pressure and disposition traces; and
in said processor, determining said medication instance confidence from said plurality of event confidences.

6. The method of claim 1, comprising:
in said processor, determining a dispensed quantity of said medication in said medication instance from said plurality of signatures; and
in response to said medication instance confidence satisfying said threshold therefor, registering said dispensed quantity with said processor; and
communicating said dispensed quantity to said recipient.

7. The method of claim 1, comprising:
in response to said medication instance confidence satisfying said threshold therefor, registering with said processor and communicating to said recipient at least one of:
said medication instance confidence;
said signatures; and
said pressure and disposition traces.

8. The method of claim 5, comprising:
in response to said medication instance confidence satisfying said threshold therefor, registering at least one of said event confidences and said medication instance confidence with said processor; and
communicating said at least one of said event confidences and said medication instance confidence to said recipient.

9. The method of claim 1, wherein said events comprise at least one of:
taking hold of said container;
disengaging said container from a dock;
removing said container from storage;
mixing said medication in said container;
manipulating said container to enable said container to dispense said medication;
removing a cap from said container;
disposing said container to dispense said medication;
squeezing said container to dispense said medication;
disposing said container subsequent to dispensing said medication;
replacing a cap on said container;
manipulating said container to disable said container from dispensing said medication;
storing said container;
engaging said container with said dock; and
releasing said container.

10. The method of claim 2, wherein said events comprise at least one of:
removing obstruction from said outlet; and
obstructing said outlet.

11. The method of claim 3, wherein said events comprise:
a change in said quantity of said medication in said medication container.

12. The method of claim 4, wherein said events comprise:
said emergence of said medication from said medication container.

13. The method of claim 1, wherein:
said medication comprises a topical eye medication; and
said medication instance comprises delivering a drop of said medication to an eye of a subject.

14. The method of claim 13, wherein said events comprise at least one of:
disposing said container above said eye such that said drop of said medication dispensed from said container falls therein;
manipulating said container to remove a cap thereof so as to enable said drop to be dispensed from said container;
squeezing said container to cause said drop to be dispensed from said container;
shaking said container so as to mix said medication therein;
disposing said container from above said eye; and
manipulating said container to engage said cap thereof.

15. The method of claim 1, comprising at least one of:
considering an order of said signatures in determining said medication instance confidence; and
considering a repetition of said signatures in determining said medication instance confidence.

16. The method of claim 1, wherein:
registering said medication instance and medication instance time comprises recording said medication instance and said medication instance time in a data store in communication with said processor.

17. The method of claim 1, comprising:
retaining said signatures;
considering said signatures in at least one of:
identifying subsequent signatures from subsequent pressure and disposition traces; and
assigning a subsequent medication instance confidence for a plurality of subsequent signatures.

18. The method of claim 17, comprising:
applying artificial intelligence to said signatures so as to improve said at least one of identifying said subsequent signatures and assigning said subsequent medication instance confidence.

19. The method of claim 1, comprising:
accepting a manual input;
registering said manual input as said medication instance and a time of said manual input as said medication instance time.

20. The method of claim 19, comprising:
registering said manual input as said dispensation comprises flagging said dispensation as entered through said manual input.

21. The method of claim 1, wherein:
said disposition values comprise at least one of orientation values, position values, rotation values, translation values, angular acceleration values, and linear acceleration values.

22. An apparatus, comprising:
a pressure sensor adapted to engage with a medication container;
a disposition sensor adapted to engage with said medication container;
a processor in communication with said pressure sensor and said disposition sensor;
a communicator in communication with said processor;
wherein:
said pressure sensor is adapted to detect a pressure trace comprising a plurality of pressure values over time corresponding with an application of pressure to said medication container over time;
said disposition sensor is adapted to detect a disposition trace comprising a plurality of disposition values over time corresponding with a disposition of said medication container over time;
said processor is adapted to identify a plurality of signatures from said pressure and disposition traces, and to identify at least one of said signatures from said pressure and disposition traces in cooperation, each said signature being consistent with a corresponding event in a medication instance, said medication instance comprising dispensing a medication from said medication container;
said processor is adapted to determine a medication instance confidence that said plurality of signatures collectively represents said medication instance based on said signatures;
said processor is adapted to determine whether said medication instance confidence satisfies a threshold therefor;
said processor is adapted to register said medication instance and a medication instance time if said medication instance confidence satisfies said threshold;
said communicator is adapted to communicate said medication instance and said medication instance time to a recipient.

23. The apparatus of claim 22, wherein:
said pressure sensor and said disposition sensor are engaged with a jacket, said jacket being engaged with said medication bottle.

24. The apparatus of claim 23, wherein:
said jacket comprises a shell around at least a portion of said medication bottle, and is removably engaged with said medication bottle.

25. The apparatus of claim 23, wherein:
said jacket comprises a label around at least a portion of said medication bottle, and is fixedly engaged with said medication bottle.

26. The apparatus of claim 22, wherein:
said pressure sensor and said disposition sensor are integrally engaged with said medication container.

27. The apparatus of claim 26, wherein:
said pressure sensor and said disposition sensor are disposed within a wall of said medication container.

28. The apparatus of claim 22, wherein:
said processor and said communicator are disposed within a shoe removably engaged with a foot of said medication container.

29. An apparatus comprising:
means for detecting a pressure trace comprising a plurality of pressure values over time corresponding with an application of pressure to said medication container over time;
means for detecting a disposition trace comprising a plurality of disposition values over time corresponding with a disposition of said medication container over time;
means for communicating said pressure and disposition traces to a processor;
means for identifying a plurality of signatures from said pressure and disposition traces, identifying at least one of said signatures from said pressure and disposition traces in cooperation, each said signature being consistent with a corresponding event in a medication instance, said medication instance comprising dispensing a medication from said medication container;
means for determining a medication instance confidence that said plurality of signatures collectively represents said medication instance based on said signatures;
means for registering said medication instance and a medication instance time in response to said medication instance confidence satisfying a threshold therefor; and
means for communicating said medication instance and said medication instance time to a recipient.
